(19) United States
(12) Patent Application Publication
(10) Pub. No.: US 2023/0183708 A1
(43) Pub. Date: Jun. 15, 2023

(54) PD-1-specific antisense oligonucleotide and its use in therapy

(71) Applicant: Secarna Pharmaceuticals GmbH & Co. KG, Marburg (DE)

(72) Inventors: Richard KLAR, München (DE); Sven MICHEL, Bernried (DE); Frank JASCHINSKI, Puchheim (DE)

(21) Appl. No.: 17/919,611

(22) PCT Filed: Apr. 30, 2021
(86) PCT No.: PCT/EP2021/061380

§ 371 Date: Oct. 18, 2022

(30) Foreign Application Priority Data
Apr. 30, 2020 (EP) 20172314.5

(51) Int. Cl. C12N 15/113; A61P 35/00

(52) U.S. Cl. CPC C12N 15/1138; A61P 35/00; C12N 2310/11; C12N 2310/315; C12N 2310/3231

(57) ABSTRACT

The present invention refers to an antisense oligonucleotide comprising 10 to 25 nucleotides, wherein at least one of the nucleotides is modified, and the antisense oligonucleotide hybridizes with a nucleic acid sequence of Programmed Cell Death 1 (PD-5 1) of SEQ ID NO.1, wherein the antisense oligonucleotide inhibits at least 30% of the PDl expression in a cell compared to an untreated cell. The invention further refers to a pharmaceutical composition comprising such antisense oligonucleotide as well as the use of the antisense oligonucleotide or the pharmaceutical composition in a method of preventing and/or treating a malignant tumor, a benign tumor and/or an infectious disease. The antisense oligonucleotide or the pharmaceutical composition is alternatively used for reducing expression of PD-1 in an isolated immune cell in preparation for cell therapy.

The present invention refers to an antisense oligonucleotide comprising 10 to 25 nucleotides hybridizing with a nucleic acid sequence of Programmed Cell Death 1 (PD-1) of SEQ ID NO.1, wherein at least one nucleotide is modified, and inhibiting the expression of PD-1. The invention is further directed to a pharmaceutical composition comprising an antisense oligonucleotide of the present invention, wherein the antisense oligonucleotide and the pharmaceutical composition, respectively, is used in a method of preventing and/or treating a malignant tumor, a benign tumor and/or an infectious disease. In addition, the antisense oligonucleotide or the pharmaceutical composition is further used in reducing expression of PD-1 RNA in an isolated immune cell for use in cell therapy.

### TECHNICAL BACKGROUND

PD-1 is a type I transmembrane protein preferentially expressed in immune cells such as T, B and NK cells. Programmed cell death 1 ligand 1 (PD-L1) is a member of the B7 family of co-stimulatory/co-inhibitory molecules of antigen presentation expressed by a wide range of cell types, including cancer cells. When engaged to its receptor PD-L1, PD1 strongly interferes with T cell receptor (TCR) signal transduction through several poorly understood molecular mechanisms. PD1 is made of an extracellular immunoglobulin-like binding domain, a transmembrane region and a cytoplasmic domain containing an immunoreceptor tyrosine-based inhibitory motif (ITIM) and an immunoreceptor tyrosine-based switch motif (ITSM). These motifs are implicated in its immunosuppressive effects. Interfering with PD1 signal transduction either by antibody blockade or any other means enhances T cell functions by potentiating signal transduction from the TCR signalosome.

PD-1 (encoded by the PDCD1 gene) plays a particularly important role in the suppression of T cell responses. After activation, the expression of PD-1 is induced on the surface of T cells. Within the framework of an antigen-specific T cell response, various activating factors are phosphorylated by binding the T cell receptor (in the case of CAR, T cells, e.g., a CAR). By binding PD-1 to its ligand PD-L1, this phosphorylation is counteracted, resulting in reduced secretion of cytokines, prevention of cell division and reduced expression of survival factors. This mechanism could lead to exhaustion of the cells and to a reduced therapeutic efficiency in the context of T cell therapies.

In recent years, T cell therapies have proven to be a promising therapeutic option for patients with various diseases, especially in form of chimeric antigen receptor transgenic T cells for the treatment of cancer patients. After activation, e.g. by recognizing a surface structure on cancer cells, T cells highly upregulate the expression of genes that are supposed to limit the activity of the T cells in order to counteract and confine, respectively, an excessive response. In the context of T cell therapies, however, this can lead to the T cells not being sufficiently efficient or their persistence in the patient being reduced so that, for example, the cancer cells cannot be successfully eliminated. One of these genes is PDCD1 which codes for the protein PD-1. The interaction of PD-1 on T cells with its ligand PD-L1 on target cells limits the activity of the T cells.

Potential applications of T cell therapies include treating cancers, autoimmune disease, and infectious disease, or improving a weakened immune system.

The downsides are unsatisfying activities and thus, unsatisfying results of the different cell therapies. Thus, there is an urgent need to develop cell therapies having reduced side effects and increased efficiency.

So far, T cells have been transfected with siRNA, however, T cells are difficult to be transfected and suitable delivery reagents are lacking. One possible transfection method is electroporation, which has though a strong impact on the viability of the cells like delivery reagents. Alternatively, T cells have been treated with self-delivering siRNA (sdRNA) molecules which are complex, strongly modified molecules, whose effectiveness on desired targets is poorly characterized. Negative effects of sdRNA on cell viability have been confirmed.

In another alternative permanent removal of PDCD1 (e.g., via CRISPR/CAS) may be considered, but the permanent knockout of PDCD1 for example in therapeutic T cells bears high risks such as the development of cell tumors. These risks can be avoided by temporary inhibition of the PD-1 expression.

Hence, an antisense oligonucleotide is missing which is highly efficient in reduction and inhibition, respectively, of PD-1 expression. siRNA and sdRNA bear the above mentioned risks of poor efficacy and/or side effects as well as permanent removal of PDCD1.

An antisense oligonucleotide of the present invention is very successful in the inhibition of the expression of PD-1 and overcomes the previously mentioned problems. Moreover, the mode of action of an antisense oligonucleotide differs from the mode of action of an antibody or small molecule, and antisense oligonucleotides are highly advantageous regarding for example
(i) the penetration of tumor tissue in solid tumors,
(ii) the use in cell therapy including ex vivo treatment of immune cells resulting in non-permanent long-term in vivo effects,
(iii) the combination of oligonucleotides with each other or an antibody or a small molecule, and
(iv) the inhibition of intracellular effects which are not accessible for an antibody or inhibitable via a small molecule.

### SUMMARY OF THE INVENTION

The present invention is directed to an antisense oligonucleotide comprising 10 to 25 nucleotides, wherein at least one of the nucleotides is modified, and the antisense oligonucleotide hybridizes with a nucleic acid sequence of Programmed Cell Death 1 (PD-1) of SEQ ID NO.1 (NG_012110.1:5001-14026 Homo sapiens programmed cell death 1 (PDCD1), RefSeqGene on chromosome 2), wherein the antisense oligonucleotide inhibits at least 30% of the PD1 expression in a cell compared to an untreated cell. The modified nucleotide is for example selected from the group consisting of a bridged nucleic acid such as LNA, cET, ENA, 2′Fluoro modified nucleotide, 2O-Methyl modified nucleotide and a combination thereof. The modified nucleotide(s) is/are for example located at the 5′- or 3′-end, at the 5′- and 3′-end of the oligonucleotide, within the antisense oligonucleotide or a combination thereof.

The antisense oligonucleotide of the present invention hybridizes for example within the region of from position 0 to position 299 of SEQ ID NO.1, within the region of from position 300 to position 599 of SEQ ID NO.1, within the region of from position 600 to position 899 of SEQ ID NO.1, within the region of from position 900 to position 1199 of SEQ ID NO.1, within the region of from position 1200 to position 1499 of SEQ ID NO.1, within the region of from position 1500 to position 1799 of SEQ ID NO.1, within the region of from position 1800 to position 2099 of SEQ ID NO.1, within the region of from position 2100 to position 2399 of SEQ ID NO.1, within the region of from position 2400 to position 2699 of SEQ ID NO.1, within the region of from position 2700 to position 2999 of SEQ ID NO.1, within the region of from position 3000 to position 3299 of SEQ ID NO.1, within the region of from position 3300 to position 3599 of SEQ ID NO.1, within the region of from position 3600 to position 3899 of SEQ ID NO.1, within the region of from position 3900 to position 4199 of SEQ ID NO.1, within the region of from position 4200 to position 4499 of SEQ ID NO.1, within the region of from position 4500 to position 4799 of SEQ ID NO.1, within the region of from position 4800 to position 5099 of SEQ ID NO.1, within the region of from position 5100 to position 5399 of SEQ ID NO.1, within the region of from position 5400 to position 5699 of SEQ ID NO.1, within the region of from position 5700 to position 5999 of SEQ ID NO.1, within the region of from position 6000 to position 6299 of SEQ ID NO.1, within the region of from position 6300 to position 6599 of SEQ ID NO.1, within the region of from position 6600 to position 6899 of SEQ ID NO.1, within the region of from position 6900 to position 7199 of SEQ ID NO.1, within the region of from position 7200 to position 7499 of SEQ ID NO.1, within the region of from position 7500 to position 7799 of SEQ ID NO.1, within the region of from position 7800 to position 8099 of SEQ ID NO.1, within the region of from position 8100 to position 8399 of SEQ ID NO.1, within the region of from position 8400 to position 8699 of SEQ ID NO.1, within the region of from position 8700 to position 8999 of SEQ ID NO.1 or within the region of from position 9000 to position 9299 of SEQ ID NO.1 or a combination thereof.

The antisense oligonucleotide of the present invention comprises for example a sequence selected from the group consisting of SEQ ID NO.22, SEQ ID NO.27, SEQ ID NO.29, SEQ ID NO. 18, SEQ ID NO.20, SEQ ID NO. 16, SEQ ID NO. 14, SEQ ID NO.34, SEQ ID NO.42, SEQ ID NO.20, SEQ ID NO.23, SEQ ID NO.40 and a combination thereof.

The antisense oligonucleotide of the present invention is further selected for example from the group consisting of

+C*+G*+T*C*G*T*A*A*A*G*C*C*A*A*+G*+G*+T (SEQ ID NO.22; A37024HI);
+T*+G*+A*G*A*G*T*C*T*T*G*T*C*C*+G*+G*+C (SEQ ID NO.27; A37030HI);
+C*+G*+A*A*T*G*G*C*G*A*A*C*G*C*+A*+G*+T (SEQ ID NO.29; A37032HI);
+T*+G*+G*A*C*G*G*C*C*T*G*C*A*A*+T*+G*+G (SEQ ID NO.18; A37019HI);
+G*G*+A*A*C*G*C*C*T*G*T*A*C*C*+T*+T (SEQ ID NO.20; A37021HI);
+C*+A*+T*A*C*T*C*C*G*T*C*T*G*C*+T*+C*+A (SEQ ID NO.16; A37017HI);
+C*+T*+T*T*G*A*T*C*T*G*C*G*C*C*+T*+T*+G (SEQ ID NO.14; A37015HI);
+C*G*+G*C*A*T*C*T*C*T*G*A*C*C*G*+T*+G (SEQ ID NO.34; A37037HI);
+C*+G*+A*G*A*T*G*C*C*A*T*G*C*A*+A*+C*+G (SEQ ID NO.42; A37046HI);
+G*G*+A*A*C*G*C*C*T*G*T*A*C*C*+T*+T (SEQ ID NO.20; A37022HI);
+G*+A*+A*C*T*G*T*C*C*T*C*A*C*T*+C*+G*+A (SEQ ID NO.23; A37025HI);
+G*+C*+T*G*A*C*A*A*G*C*G*C*T*C*G*+C*+C (SEQ ID NO.40; A37043HI)

and a combination thereof, wherein + indicates a LNA-modified nucleotide and * indicates phosphorothioate.

The present invention further refers to a pharmaceutical composition comprising an antisense oligonucleotide of the present invention and a pharmaceutically acceptable excipient.

The antisense oligonucleotide and the pharmaceutical composition, respectively, of the present invention are for example for use in T cell therapy. The antisense oligonucleotide or the pharmaceutical composition of the present invention are in further examples for use in a method of preventing and/or treating a malignant tumor, a benign tumor and/or an infectious disease.

The tumor is for example selected from the group consisting of solid tumors, blood born tumors, leukemias, tumor metastasis, hemangiomas, acoustic neuromas, neurofibromas, trachomas, pyogenic granulomas, psoriasis, astrocytoma, blastoma, Ewing’s tumor, craniopharyngioma, ependymoma, medulloblastoma, glioma, hemangioblastoma, Hodgkin’s lymphoma, mesothelioma, neuroblastoma, non-Hodgkin’s lymphoma, pinealoma, retinoblastoma, sarcoma, seminoma, and Wilms’ tumor, bile duct carcinoma, bladder carcinoma, brain tumor, breast cancer, bronchogenic carcinoma, carcinoma of the kidney, cervical cancer, choriocarcinoma, choroid carcinoma, cystadenocarcinoma, embryonal carcinoma, epithelial carcinoma, esophageal cancer, cervical carcinoma, colon carcinoma, colorectal carcinoma, endometrial cancer, gallbladder cancer, gastric cancer, head cancer, liver carcinoma, lung carcinoma, medullary carcinoma, neck cancer, non-small-cell bronchogenic/lung carcinoma, ovarian cancer, pancreas carcinoma, papillary carcinoma, papillary adenocarcinoma, prostate cancer, small intestine carcinoma, prostate carcinoma, rectal cancer, renal cell carcinoma, skin cancer, small-cell bronchogenic/lung carcinoma, squamous cell carcinoma, sebaceous gland carcinoma, testicular carcinoma, uterine cancer or a combination thereof.

The infectious disease is for example selected from the group consisting of a Hepatitis B infection, a Hepatitis A infection, a Cytomegalovirus infection, an Epstein-Barr-Virus infection, an Adenovirus infection or a combination thereof.

The antisense oligonucleotide or the pharmaceutical composition of the present invention is for example used in reducing expression of PD-1 RNA in an isolated immune cell in preparation for cell therapy.

In addition, the present invention refers to a method for reducing expression of PD-1 RNA in an isolated immune cell in preparation for cell therapy, comprising: incubating the isolated immune cell comprising the PD-1 RNA with an antisense oligonucleotide or the pharmaceutical composition of the present invention without use of a transfection means, wherein the antisense oligonucleotide is administered to the isolated immune cell at least once in a time period of day 0 to day 21, the antisense oligonucleotide hybridizes with the PD-1 RNA and reduces the expression of PD-1 (of e.g., RNA), reduces the function and/or activity of PD-1 (of e.g., protein), or a combination thereof up to 8 weeks from day 0 of the incubation with the antisense oligonucleotide. The isolated immune cell is for example genetically modified by a gene transfer technology before or after incubating the immune cell with the antisense oligonucleotide. The genetically modification of the immune cell is for example permanent or transient. The isolated, genetically modified immune cell is for example expanded before or after incubating the immune cell with the antisense oligonucleotide. The immune cell is for example permanently or transiently genetically modified. The immune cell is for example selected from the group consisting of a T cell, a dendritic cell, a natural killer (NK) cell, a peripheral blood mononuclear cell (PBMC), a hematopoietic stem cell, a B cell and a combination thereof.

All documents cited or referenced herein (“herein cited documents”), and all documents cited or referenced in herein cited documents, together with any manufacturer’s instructions, descriptions, product specifications, and product sheets for any products mentioned herein or in any document incorporated by reference herein, are hereby incorporated herein by reference, and may be employed in the practice of the invention.

More specifically, all referenced documents are incorporated by reference to the same extent as if each individual document was specifically and individually indicated to be incorporated by reference.

### DESCRIPTION OF THE FIGURES

FIG. 1 depicts a schematic of a T cell therapy.

FIGS. 2A and 2B depict efficacy screening of PD-1 ASOs in activated human T cells.

FIG. 3 shows the dose-dependent PD-1 mRNA knockdown by two selected PD-1 ASOs in activated human T cells.

FIGS. 4A to 4C show time-dependency of PD-1 knockdown in activated human T cells after treatment with selected PD-1 ASOs, wherein FIG. 4A refers to PD-1 mRNA expression, FIG. 4B to % PD-1+ cells in Life gate and FIG. 4C to residual % PD-1 cells in Life gate.

FIGS. 5A and 5B depict persistency of PD-1 target knockdown in activated human T cells after ASO treatment, stringent washing and re-stimulation, wherein FIG. 5A shows residual PD-1 mRNA expression and FIG. 5B shows residual % PD-1 cells in Life gate.

FIGS. 6A and 6B show comparison of the effects of a PD-1-specific ASO and a PD-1 specific self-delivering small interfering RNA in activated human T cells, wherein FIG. 6A depicts residual PD-1 mRNA expression and FIG. 6B depicts relative viability as compared to mock-treated cells.

### DETAILED DESCRIPTION

The present invention provides for the first time human and murine antisense oligonucleotides which hybridize with a pre-mRNA sequence of Programmed Cell Death 1 (PD-1) of SEQ ID NO.1 (NG_012110.1:5001-14026 Homo sapiens programmed cell death 1 (PDCD1), RefSeqGene on chromosome 2) and inhibit the expression, function and/or activity, of PD-1. Pre-mRNA comprises exons, introns and UTRs of the PD-1 encoding nucleic acid sequence. Thus, the oligonucleotides of the present invention represent an interesting and highly efficient tool for use in a T cell therapy and a method of preventing and/or treating disorders, respectively, where the PD-1 expression, function and /or activity is not desired or increased.

Reducing expression of a PD-1 RNA according to the present invention means decreasing the expression (of e.g., RNA), function and/or activity of the PD-1 (of e.g., protein) in different amounts up to complete inhibition. Thus, the PD-1 protein is not or only in a reduced amount available to a cell. The expression, function and/or activity level in the cell is determined for example by measuring and comparing the expression, function and/or activity level of the PD-1 before treatment, i.e., administration of an oligonucleotide, and after treatment.

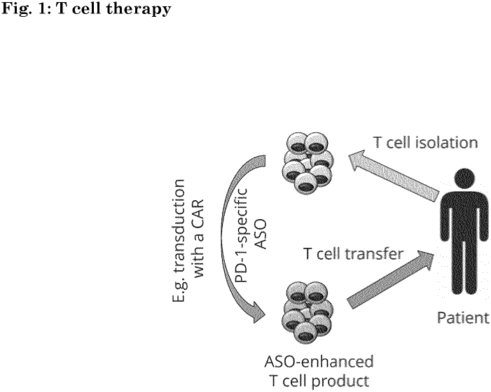

The antisense oligonucleotides of the present invention are for example designed in silico and examined in vitro for their mRNA and protein knockdown efficiency. They are suitable for the production of T cell products, wherein T cells are for example isolated from a patient (or an allogeneic donor), genetically modified ex vivo (e.g., with a CAR) if necessary, expanded and treated with the PD-1 antisense oligonucleotides during the ex vivo phase of the production. The cells are then (re-)transferred to the patient. If the T cells encounter tumor cells and recognize a corresponding target structure, they are activated. The persistence of PD-1 antisense oligonucleotides prevents the upregulation of PD-1 expression during the encounter with tumor cells (see e.g., FIG. 1).

In the following, the elements of the present invention will be described in more detail. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by the context. In particular, the terms “a” and “an” and “the” are synonymous to “one or more”. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the invention and does not pose a limitation on the scope of the invention otherwise claimed. No language in the specification should be construed as indicating any non-claimed element essential to the practice of the invention.

An oligonucleotide of the present invention is for example an antisense oligonucleotide (ASO) consisting of or comprising 10 to 25 nucleotides, 10 to 15 nucleotides, 15 to 20 nucleotides, 12 to 19 nucleotides, or 15 to 18 nucleotides. The oligonucleotides for example consist of or comprise 10, 11, 12, 13, 14, 15, 16, 17, 18, 19, 20, 21, 22, 23, 24 or 25 nucleotides. The oligonucleotides of the present invention comprise at least one nucleotide which is modified. The modified nucleotide is for example a bridged nucleotide such as a locked nucleic acid (LNA, e.g., 2′,4′-LNA), cET, ENA, a 2′Fluoro modified nucleotide, a 2‛O-Methyl modified nucleotide or a combination thereof. In some embodiments, the oligonucleotide of the present invention comprises nucleotides having the same or different modifications. In some embodiments the oligonucleotide of the present invention comprises a modified phosphate backbone, wherein the phosphate is for example a phosphorothioate.

The antisense oligonucleotide of the present invention is for example an antisense oligonucleotide, siRNA, sdRNA or aptamer.

The oligonucleotide of the present invention comprises the one or more modified nucleotides at the 3′- and/or 5′- end of the oligonucleotide and/or at any position within the oligonucleotide, wherein modified nucleotides follow in a row of 1, 2, 3, 4, 5, or 6 modified nucleotides, or a modified nucleotide is combined with one or more unmodified nucleotides. The following Table 1 presents embodiments of oligonucleotides comprising modified nucleotides for example LNA which are indicated by (+) and phosphorothioate (PTO) indicated by (*); alternatively, the phosphate backbone of the antisense oligonucleotide is unmodified. The oligonucleotides consisting of or comprising the sequences of Table 1 may comprise any other modified nucleotide and/or any other combination of modified and unmodified nucleotides. Oligonucleotides of Table 1 hybridize with pre-mRNA of PD-1 of SEQ ID NO.1:

| Seq ID | Name | Antisense Sequence 5′-3′ | Antisense Sequence 5′-3′ with PTO (*) and LNA (+) |
|---|---|---|---|
| 2 | A37001H | CACCAGAGTGCCGCCTT | +C*+A*C*C*A*G*A*G*T*G*C*C*G*C*+C*+T*+T |
| 2 | A37002H | CACCAGAGTGCCGCCTT | +C*+A*C*C*A*G*A*G*T*G*C*C*G*C*+C*T*+T |
| 2 | A37003H | CACCAGAGTGCCGCCTT | +C*+A*C*C*A*G*A*G*T*G*C*C*G*C*C*+T*+T |
| 3 | A37004H | CGGTCACCACGAGCAGG | +C*+G*+G*T*C*A*C*C*A*C*G*A*G*C*+A*+G*+G |
| 4 | A37005H | TCGGTCACCACGAGCAG | +T*+C*+G*G*T*C*A*C*C*A*C*G*A*G*+C*+A*+G |
| 5 | A37006H | CCTTCGGTCACCACGA | +C*+C*+T*T*C*G*G*T*C*A*C*C*A*+C*+G*+A |
| 6 | A37007H | CGAAGCTCTCCGATGTG | +C*+G*+A*A*G*C*T*C*T*C*C*G*A*T*+G*+T*+G |
| 7 | A37008H | GCACGAAGCTCTCCGAT | +G*+C*+A*C*G*A*A*G*C*T*C*T*C*C*+G*+A*+T |
| 8 | A37009H | CCAGTTTAGCACGAAGC | +C*+C*+A*G*T*T*T*A*G*C*A*C*G*A*+A*+G*+C |
| 9 | A37010H | GTACCAGTTTAGCACGA | +G*+T*+A*C*C*A*G*T*T*T*A*G*C*A*+C*+G*+A |
| 10 | A37011H | ATGCGGTACCAGTTTAG | +A*+T*+G*C*G*G*T*A*C*C*A*G*T*T*+T*+A*+G |
| 11 | A37012HM | CTTGTCCGTCTGGTTGC | +C*+T*+T*G*T*C*C*G*T*C*T*G*G*T*+T*+G*+C |
| 12 | A37013H | TGACACGGAAGCGGCAG | +T*+G*+A*C*A*C*G*G*A*A*G*C*G*G*+C*+A*+G |
| 13 | A37014H | ACAGAGGTAGGTGCCGC | +A*+C*+A*G*A*G*G*T*A*G*G*T*G*C*+C*+G*+C |
| 14 | A37015HM | CTTTGATCTGCGCCTTG | +C*+T*+T*T*G*A*T*C*T*G*C*G*C*C*+T*+T*+G |
| 15 | A37016HM | ACGACACCAACCACCAG | +A*+C*+G*A*C*A*C*C*A*A*C*C*A*C*+C*+A*+G |
| 16 | A37017H | CATACTCCGTCTGCTCA | +C*+A*+T*A*C*T*C*C*G*T*C*T*G*C*+T*+C*+A |
| 17 | A37018H | CCATTCCGCTAGGAAAG | +C*+C*+A*T*T*C*C*G*C*T*A*G*G*A*+A*+A*+G |
| 18 | A37019H | TGGACGGCCTGCAATGG | +T*+G*+G*A*C*G*G*C*C*T*G*C*A*A*+T*+G*+G |
| 19 | A37020H | CCACGGCGCCTTCAGCC | +C*C*+A*C*G*G*C*G*C*C*T*T*C*A*+G*+C*+C |
| 20 | A37021H | GGAACGCCTGTACCTT | +G*+G*+A*A*C*G*C*C*T*G*T*A*C*C*+T*+T |
| 20 | A37022H | GGAACGCCTGTACCTT | +G*G*+A*A*C*G*C*C*T*G*T*A*C*C*+T*+T |
| 21 | A37023HI | TCGAGTGAGGACCAAGG | +T*+C*+G*A*G*T*G*A*G*G*A*C*C*A*+A*+G*+G |
| 22 | A37024HI | CGTCGTAAAGCCAAGGT | +C*+G*+T*C*G*T*A*A*A*G*C*C*A*A*+G*+G*+T |
| 23 | A37025HI | GAACTGTCCTCACTCGA | +G*+A*+A*C*T*G*T*C*C*T*C*A*C*T*+C*+G*+A |
| 24 | A37026HI | CTCTCTCCTCGATCCGG | +C*+T*C*T*C*T*C*C*T*C*G*A*T*C*+C*G*+G |
| 25 | A37027HI | CGTGCCTGAAGAGCCGG | +C*G*+T*G*C*C*T*G*A*A*G*A*G*C*C*+G*+G |
| 26 | A37028HI | TGTCCGGCACAAGCGCG | +T*+G*+T*C*C*G*G*C*A*C*A*A*G*C*G*+C*+G |
| 26 | A37029HI | TGTCCGGCACAAGCGCG | +T*+G*T*C*C*G*G*C*A*C*A*A*G*C*+G*C*+G |
| 27 | A37030HI | TGAGAGTCTTGTCCGGC | +T*+G*+A*G*A*G*T*C*T*T*G*T*C*C*+G*+G*+C |
| 28 | A37031HI | AACGCAGTGAATAGATC | +A*+A*+C*G*C*A*G*T*G*A*A*T*A*G*+A*+T*+C |
| 29 | A37032HI | CGAATGGCGAACGCAGT | +C*+G*+A*A*T*G*G*C*G*A*A*C*G*C*+A*+G*+T |
| 30 | A37033HI | AAGTCCTGTCGAAGGCC | +A*+A*G*T*C*C*T*G*T*C*G*A*A*G*+G*C*+C |
| 31 | A37034HI | AGCCACTCGGTCGGCGG | +A*+G*C*C*A*C*T*C*G*G*T*C*G*G*C*+G*+G |
| 32 | A37035HI | AAGCCACTCGGTCGGCG | +A*+A*G*C*C*A*C*T*C*G*G*T*C*G*+G*C*+G |
| 33 | A37036HI | GCGGCCTTATTAGGAAT | +G*+C*+G*G*C*C*T*T*A*T*T*A*G*G*+A*+A*+T |
| 34 | A37037HI | CGGCATCTCTGACCGTG | +C*G*+G*C*A*T*C*T*C*T*G*A*C*C*G*+T*+G |
| 35 | A37038HI | CGCAGAGAGACCGCATT | +C*+G*+C*A*G*A*G*A*G*A*C*C*G*C*+A*+T*+T |
| 36 | A37039HI | CTCGGATCCACGTAGGA | +C*+T*+C*G*G*A*T*C*C*A*C*G*T*A*+G*+G*+A |
| 37 | A37040HI | TGATCTGTGCTGGCGCT | +T*+G*A*T*C*T*G*T*G*C*T*G*G*C*+G*C*+T |
| 38 | A37041HI | CGACAGGACAATGGCCG | +C*+G*A*C*A*G*G*A*C*A*A*T*G*G*+C*C*+G |
| 39 | A37042HI | ATAGGCGTGTGCGGCGT | +A*+T*+A*G*G*C*G*T*G*T*G*C*G*G*+C*+G*+T |
| 40 | A37043HI | GCTGACAAGCGCTCGCC | +G*+C*+T*G*A*C*A*A*G*C*G*C*T*C*G*+C*+C |
| 40 | A37044HI | GCTGACAAGCGCTCGCC | +G*+C*T*G*A*C*A*A*G*C*G*C*T*C*G*+C*+C |
| 41 | A37045HI | GGACCAACTCCTAGTGC | +G*+G*+A*C*C*A*A*C*T*C*C*T*A*G*+T*+G*+C |
| 42 | A37046HI | CGAGATGCCATGCAACG | +C*+G*+A*G*A*T*G*C*C*A*T*G*C*A*+A*+C*+G |
| 43 | A37047HI | CTTCTACGTGAGGCTGC | +C*+T*+T*C*T*A*C*G*T*G*A*G*G*C*+T*+G*+C |
| 44 | A37048HI | TGAGCCGTGCTCCTAGG | +T*G*+A*G*C*C*G*T*G*C*T*C*C*T*+A*G*+G |
| 45 | A37049HI | TACCGGCACCGAACCTG | +T*+A*+C*C*G*G*C*A*C*C*G*A*A*C*C*+T*+G |
| 45 | A37050HI | TACCGGCACCGAACCTG | +T*+A*C*C*G*G*C*A*C*C*G*A*A*C*C*+T*+G |
| 46 | A37051HI | TACCGGCACCGAACCT | +T*A*+C*C*G*G*C*A*C*C*G*A*A*+C*C*+T |
| 47 | A37052HI | GCAGTACCGGCACCGA | +G*+C*+A*G*T*A*C*C*G*G*C*A*C*+C*G*+A |
| 48 | A37053H | TCGGTCACCACGAGCAGG | +T*+C*+G*G*T*C*A*C*C*A*C*G*A*G*C*+A*+G*+G |
| 49 | A37054H | CCTTCGGTCACCACGAGC | +C*+C*+T*T*C*G*G*T*C*A*C*C*A*C*G*+A*+G*+C |
| 50 | A37055H | AGGTGAAGGTGGCGTTGT | +A*+G*+G*T*G*A*A*G*G*T*G*G*C*G*T*+T*+G*+T |
| 51 | A37056H | CAGGTGAAGGTGGCGTTG | +C*+A*+G*G*T*G*A*A*G*G*T*G*G*C*G*+T*+T*+G |
| 52 | A37057H | AAGCTCTCCGATGTGTTG | +A*+A*+G*C*T*C*T*C*C*G*A*T*G*T*G*+T*+T*+G |
| 53 | A37058H | CGAAGCTCTCCGATGTGT | +C*+G*+A*A*G*C*T*C*T*C*C*G*A*T*G*+T*+G*+T |
| 54 | A37059H | AGCACGAAGCTCTCCGAT | +A*+G*+C*A*C*G*A*A*G*C*T*C*T*C*C*+G*+A*+T |
| 55 | A37060H | ACCAGTTTAGCACGAAGC | +A*+C*+C*A*G*T*T*T*A*G*C*A*C*G*A*+A*+G*+C |
| 56 | A37061H | TACCAGTTTAGCACGAAG | +T*+A*+C*C*A*G*T*T*T*A*G*C*A*C*G*+A*+A*+G |
| 57 | A37062H | GTACCAGTTTAGCACGAA | +G*+T*+A*C*C*A*G*T*T*T*A*G*C*A*C*+G*+A*+A |
| 58 | A37063H | CATGCGGTACCAGTTTAG | +C*+A*+T*G*C*G*G*T*A*C*C*A*G*T*T*+T*+A*+G |
| 59 | A37064H | TCATGCGGTACCAGTTTA | +T*+C*+A*T*G*C*G*G*T*A*C*C*A*G*T*+T*+T*+A |
| 60 | A37065H | TTGTCCGTCTGGTTGCTG | +T*+T*+G*T*C*C*G*T*C*T*G*G*T*T*G*+C*+T*+G |
| 61 | A37066H | CTTGTCCGTCTGGTTGCT | +C*+T*+T*G*T*C*C*G*T*C*T*G*G*T*T*+G*+C*+T |
| 62 | A37067H | TGTGACACGGAAGCGGCA | +T*+G*+T*G*A*C*A*C*G*G*A*A*G*C*G*+G*+C*+A |
| 63 | A37068H | CAGTTGTGTGACACGGAA | +C*+A*+G*T*T*G*T*G*T*G*A*C*A*C*G*+G*+A*+A |
| 64 | A37069H | GCAGTTGTGTGACACGGA | +G*+C*+A*G*T*T*G*T*G*T*G*A*C*A*C*+G*+G*+A |
| 65 | A37070H | TCATGTGGAAGTCACGCC | +T*+C*+A*T*G*T*G*G*A*A*G*T*C*A*C*+G*+C*+C |
| 66 | A37071H | GCTCATGTGGAAGTCACG | +G*+C*+T*C*A*T*G*T*G*G*A*A*G*T*C*+A*+C*+G |
| 67 | A37072H | CACAGAGGTAGGTGCCGC | +C*+A*+C*A*G*A*G*G*T*A*G*G*T*G*C*+C*+G*+C |
| 68 | A37073H | CTTTGATCTGCGCCTTGG | +C*+T*+T*T*G*A*T*C*T*G*C*G*C*C*T*+T*+G*+G |
| 69 | A37074H | TCTTTGATCTGCGCCTTG | +T*+C*+T*T*T*G*A*T*C*T*G*C*G*C*C*+T*+T*+G |
| 70 | A37075H | CTCTCTTTGATCTGCGCC | +C*+T*+C*T*C*T*T*T*G*A*T*C*T*G*C*+G*+C*+C |
| 71 | A37076H | CGCAGGCTCTCTTTGATC | +C*+G*+C*A*G*G*C*T*C*T*C*T*T*T*G*+A*+T*+C |
| 72 | A37077H | CCGCAGGCTCTCTTTGAT | +C*+C*+G*C*A*G*G*C*T*C*T*C*T*T*T*+G*+A*+T |
| 73 | A37078H | ACGACACCAACCACCAGG | +A*+C*+G*A*C*A*C*C*A*A*C*C*A*C*C*+A*+G*+G |
| 74 | A37079H | CACGACACCAACCACCAG | +C*+A*+C*G*A*C*A*C*C*A*A*C*C*A*C*+C*+A*+G |
| 75 | A37080H | GAGAACACAGGCACGGCT | +G*+A*+G*A*A*C*A*C*A*G*G*C*A*C*G*+G*+C*+T |
| 76 | A37081H | ATAGTCCACAGAGAACAC | +A*+T*+A*G*T*C*C*A*C*A*G*A*G*A*A*+C*+A*+C |
| 77 | A37082H | CATAGTCCACAGAGAACA | +C*+A*+T*A*G*T*C*C*A*C*A*G*A*G*A*+A*+C*+A |
| 78 | A37083H | CCATAGTCCACAGAGAAC | +C*+C*+A*T*A*G*T*C*C*A*C*A*G*A*G*+A*+A*+C |
| 79 | A37084H | GGTCTTCTCTCGCCACTG | +G*+G*+T*C*T*T*C*T*C*T*C*G*C*C*A*+C*+T*+G |
| 80 | A37085H | ATACTCCGTCTGCTCAGG | +A*+T*+A*C*T*C*C*G*T*C*T*G*C*T*C*+A*+G*+G |
| 81 | A37086H | CCATTCCGCTAGGAAAGA | +C*+C*+A*T*T*C*C*G*C*T*A*G*G*A*A*+A*+G*+A |
| 82 | A37087H | CCAAGGAAGCCGGTCAGA | +C*+C*+A*A*G*G*A*A*G*C*C*G*G*T*C*+A*+G*+A |
| 83 | A37088H | CATTGAGACATGAGTCCT | +C*+A*+T*T*G*A*G*A*C*A*T*G*A*G*T*+C*+C*+T |
| 84 | A37089H | GCATTGAGACATGAGTCC | +G*+C*+A*T*T*G*A*G*A*C*A*T*G*A*G*+T*+C*+C |
| 85 | A37090H | CCTTAGCATGCTCTCATA | +C*+C*+T*T*A*G*C*A*T*G*C*T*C*T*C*+A*+T*+A |
| 86 | A37091H | CAGGCGGAGGTGAGCGGAA | +C*+A*+G*G*C*G*G*A*G*G*T*G*A*G*C*G*+G*+A*+A |
| 87 | A37092H | GTGCCGCCTTCTCCACTGC | +G*+T*+G*C*C*G*C*C*T*T*C*T*C*C*A*C*+T*+G*+C |
| 88 | A37093H | GATCTGCATGCCTGGAGCA | +G*+A*+T*C*T*G*C*A*T*G*C*C*T*G*G*A*+G*+C*+A |
| 89 | A37094H | TAAGAACCATCCTGGCCGC | +T*+A*+A*G*A*A*C*C*A*T*C*C*T*G*G*C*+C*+G*+C |
| 90 | A37095H | CTTCGGTCACCACGAGCAG | +C*+T*+T*C*G*G*T*C*A*C*C*A*C*G*A*G*+C*+A*+G |
| 91 | A37096H | CCTTCGGTCACCACGAGCA | +C*+C*+T*T*C*G*G*T*C*A*C*C*A*C*G*A*+G*+C*+A |
| 92 | A37097H | CAGGTGAAGGTGGCGTTGT | +C*+A*+G*G*T*G*A*A*G*G*T*G*G*C*G*T*+T*+G*+T |
| 93 | A37098H | GCAGGTGAAGGTGGCGTTG | +G*+C*+A*G*G*T*G*A*A*G*G*T*G*G*C*G*+T*+T*+G |
| 94 | A37099H | GAGAAGCTGCAGGTGAAGG | +G*+A*+G*A*A*G*C*T*G*C*A*G*G*T*G*A*+A*+G*+G |
| 95 | A37100H | GTTGGAGAAGCTGCAGGTG | +G*+T*+T*G*G*A*G*A*A*G*C*T*G*C*A*G*+G*+T*+G |
| 96 | A37101H | GTGTTGGAGAAGCTGCAGG | +G*+T*+G*T*T*G*G*A*G*A*A*G*C*T*G*C*+A*+G*+G |
| 97 | A37102H | ATGTGTTGGAGAAGCTGCA | +A*+T*+G*T*G*T*T*G*G*A*G*A*A*G*C*T*+G*+C*+A |
| 98 | A37103H | GATGTGTTGGAGAAGCTGC | +G*+A*+T*G*T*G*T*T*G*G*A*G*A*A*G*C*+T*+G*+C |
| 99 | A37104H | GAAGCTCTCCGATGTGTTG | +G*+A*+A*G*C*T*C*T*C*C*G*A*T*G*T*G*+T*+T*+G |
| 100 | A37105H | CGAAGCTCTCCGATGTGTT | +C*+G*+A*A*G*C*T*C*T*C*C*G*A*T*G*T*+G*+T*+T |
| 101 | A37106H | AGCACGAAGCTCTCCGATG | +A*+G*+C*A*C*G*A*A*G*C*T*C*T*C*C*G*+A*+T*+G |
| 102 | A37107H | TAGCACGAAGCTCTCCGAT | +T*+A*+G*C*A*C*G*A*A*G*C*T*C*T*C*C*+G*+A*+T |
| 103 | A37108H | TTTAGCACGAAGCTCTCCG | +T*+T*+T*A*G*C*A*C*G*A*A*G*C*T*C*T*+C*+C*+G |
| 104 | A37109H | TACCAGTTTAGCACGAAGC | +T*+A*+C*C*A*G*T*T*T*A*G*C*A*C*G*A*+A*+G*+C |
| 105 | A37110H | GTACCAGTTTAGCACGAAG | +G*+T*+A*C*C*A*G*T*T*T*A*G*C*A*C*G*+A*+A*+G |
| 106 | A37111H | CATGCGGTACCAGTTTAGC | +C*+A*+T*G*C*G*G*T*A*C*C*A*G*T*T*T*+A*+G*+C |
| 107 | A37112H | TCATGCGGTACCAGTTTAG | +T*+C*+A*T*G*C*G*G*T*A*C*C*A*G*T*T*+T*+A*+G |
| 108 | A37113H | CTCATGCGGTACCAGTTTA | +C*+T*+C*A*T*G*C*G*G*T*A*C*C*A*G*T*+T*+T*+A |
| 109 | A37114H | TTGTCCGTCTGGTTGCTGG | +T*+T*+G*T*C*C*G*T*C*T*G*G*T*T*G*C*+T*+G*+G |
| 110 | A37115H | CTTGTCCGTCTGGTTGCTG | +C*+T*+T*G*T*C*C*G*T*C*T*G*G*T*T*G*+C*+T*+G |
| 111 | A37116H | GCTTGTCCGTCTGGTTGCT | +G*+C*+T*T*G*T*C*C*G*T*C*T*G*G*T*T*+G*+C*+T |
| 112 | A37117H | TGTGACACGGAAGCGGCAG | +T*+G*+T*G*A*C*A*C*G*G*A*A*G*C*G*G*+C*+A*+G |
| 113 | A37118H | GTTGTGTGACACGGAAGCG | +G*+T*+T*G*T*G*T*G*A*C*A*C*G*G*A*A*+G*+C*+G |
| 114 | A37119H | GCAGTTGTGTGACACGGAA | +G*+C*+A*G*T*T*G*T*G*T*G*A*C*A*C*G*+G*+A*+A |
| 115 | A37120H | GGCAGTTGTGTGACACGGA | +G*+G*+C*A*G*T*T*G*T*G*T*G*A*C*A*C*+G*+G*+A |
| 116 | A37121H | CTCATGTGGAAGTCACGCC | +C*+T*+C*A*T*G*T*G*G*A*A*G*T*C*A*C*+G*+C*+C |
| 117 | A37122H | TGACCACGCTCATGTGGAA | +T*+G*+A*C*C*A*C*G*C*T*C*A*T*G*T*G*+G*+A*+A |
| 118 | A37123H | CCACAGAGGTAGGTGCCGC | +C*+C*+A*C*A*G*A*G*G*T*A*G*G*T*G*C*+C*+G*+C |
| 119 | A37124H | TCTTTGATCTGCGCCTTGG | +T*+C*+T*T*T*G*A*T*C*T*G*C*G*C*C*T*+T*+G*+G |
| 120 | A37125H | CTCTTTGATCTGCGCCTTG | +C*+T*+C*T*T*T*G*A*T*C*T*G*C*G*C*C*+T*+T*+G |
| 121 | A37126H | CGCAGGCTCTCTTTGATCT | +C*+G*+C*A*G*G*C*T*C*T*C*T*T*T*G*A*+T*+C*+T |
| 122 | A37127H | CACGACACCAACCACCAGG | +C*+A*+C*G*A*C*A*C*C*A*A*C*C*A*C*C*+A*+G*+G |
| 123 | A37128H | CCACGACACCAACCACCAG | +C*+C*+A*C*G*A*C*A*C*C*A*A*C*C*A*C*+C*+A*+G |
| 124 | A37129H | CAGACTAGCAGCACCAGGC | +C*+A*+G*A*C*T*A*G*C*A*G*C*A*C*C*A*+G*+G*+C |
| 125 | A37130H | AGAGAACACAGGCACGGCT | +A*+G*+A*G*A*A*C*A*C*A*G*G*C*A*C*G*+G*+C*+T |
| 126 | A37131H | CAGAGAACACAGGCACGGC | +C*+A*+G*A*G*A*A*C*A*C*A*G*G*C*A*C*+G*+G*+C |
| 127 | A37132H | GTCCACAGAGAACACAGGC | +G*+T*+C*C*A*C*A*G*A*G*A*A*C*A*C*A*+G*+G*+C |
| 128 | A37133H | ATAGTCCACAGAGAACACA | +A*+T*+A*G*T*C*C*A*C*A*G*A*G*A*A*C*+A*+C*+A |
| 129 | A37134H | CATAGTCCACAGAGAACAC | +C*+A*+T*A*G*T*C*C*A*C*A*G*A*G*A*A*+C*+A*+C |
| 130 | A37135H | CCATAGTCCACAGAGAACA | +C*+C*+A*T*A*G*T*C*C*A*C*A*G*A*G*A*+A*+C*+A |
| 131 | A37136H | TTCTCTCGCCACTGGAAAT | +T*+T*+C*T*C*T*C*G*C*C*A*C*T*G*G*A*+A*+A*+T |
| 132 | A37137H | CTTCTCTCGCCACTGGAAA | +C*+T*+T*C*T*C*T*C*G*C*C*A*C*T*G*G*+A*+A*+A |
| 133 | A37138H | CATACTCCGTCTGCTCAGG | +C*+A*+T*A*C*T*C*C*G*T*C*T*G*C*T*C*+A*+G*+G |
| 134 | A37139H | ACAATGGTGGCATACTCCG | +A*+C*+A*A*T*G*G*T*G*G*C*A*T*A*C*T*+C*+C*+G |
| 135 | A37140H | AAGACAATGGTGGCATACT | +A*+A*+G*A*C*A*A*T*G*G*T*G*G*C*A*T*+A*+C*+T |
| 136 | A37141H | AAAGACAATGGTGGCATAC | +A*+A*+A*G*A*C*A*A*T*G*G*T*G*G*C*A*+T*+A*+C |
| 137 | A37142H | GAAAGACAATGGTGGCATA | +G*+A*+A*A*G*A*C*A*A*T*G*G*T*G*G*C*+A*+T*+A |
| 138 | A37143H | ATTCCGCTAGGAAAGACAA | +A*+T*+T*C*C*G*C*T*A*G*G*A*A*A*G*A*+C*+A*+A |
| 139 | A37144H | CAAGGAAGCCGGTCAGAGG | +C*+A*+A*G*G*A*A*G*C*C*G*G*T*C*A*G*+A*+G*+G |
| 140 | A37145H | CCAAGGAAGCCGGTCAGAG | +C*+C*+A*A*G*G*A*A*G*C*C*G*G*T*C*A*+G*+A*+G |
| 141 | A37146H | GCAGAACACTGGTGGCCAA | +G*+C*+A*G*A*A*C*A*C*T*G*G*T*G*G*C*+C*+A*+A |
| 142 | A37147H | CTGAGGAAATGCGCTGACC | +C*+T*+G*A*G*G*A*A*A*T*G*C*G*C*T*G*+A*+C*+C |
| 143 | A37148H | TCTCCTGAGGAAATGCGCT | +T*+C*+T*C*C*T*G*A*G*G*A*A*A*T*G*C*+G*+C*+T |
| 144 | A37149H | AGACATGAGTCCTGTGGTG | +A*+G*+A*C*A*T*G*A*G*T*C*C*T*G*T*G*+G*+T*+G |
| 145 | A37150H | TGAGACATGAGTCCTGTGG | +T*+G*+A*G*A*C*A*T*G*A*G*T*C*C*T*G*+T*+G*+G |
| 146 | A37151H | GCATTGAGACATGAGTCCT | +G*+C*+A*T*T*G*A*G*A*C*A*T*G*A*G*T*+C*+C*+T |
| 147 | A37152H | TGGCAGGACCTGAAGCAGT | +T*+G*+G*C*A*G*G*A*C*C*T*G*A*A*G*C*+A*+G*+T |
| 148 | A37153H | TGGACGCAGGCAGCTCTGT | +T*+G*+G*A*C*G*C*A*G*G*C*A*G*C*T*C*+T*+G*+T |
| 149 | A37154H | GCAGCAGCAGAGATTCAGG | +G*+C*+A*G*C*A*G*C*A*G*A*G*A*T*T*C*+A*+G*+G |
| 150 | A37155H | CAAGGCCATCTCCAACCAG | +C*+A*+A*G*G*C*C*A*T*C*T*C*C*A*A*C*+C*+A*+G |
| 151 | A37156H | CCAAGGCCATCTCCAACCA | +C*+C*+A*A*G*G*C*C*A*T*C*T*C*C*A*A*+C*+C*+A |
| 152 | A37157H | GCTCCAAGGCCATCTCCAA | +G*+C*+T*C*C*A*A*G*G*C*C*A*T*C*T*C*+C*+A*+A |
| 153 | A37158H | GAAACTTCTCTAGGCCTGC | +G*+A*+A*A*C*T*T*C*T*C*T*A*G*G*C*C*+T*+G*+C |
| 154 | A37159H | GGCATGTGTAAAGGTGGAG | +G*+G*+C*A*T*G*T*G*T*A*A*A*G*G*T*G*+G*+A*+G |
| 155 | A37160H | CGCTTACTGCCTCAGCTTC | +C*+G*+C*T*T*A*C*T*G*C*C*T*C*A*G*C*+T*+T*+C |
| 156 | A37161H | TGGAATGCGGCGGCAGGAG | +T*+G*+G*A*A*T*G*C*G*G*C*G*G*C*A*G*+G*+A*+G |
| 157 | A37162H | GGTGGAATGCGGCGGCAGG | +G*+G*+T*G*G*A*A*T*G*C*G*G*C*G*G*C*+A*+G*+G |
| 158 | A37163H | GTGTGGATGTGAGGAGTGG | +G*+T*+G*T*G*G*A*T*G*T*G*A*G*G*A*G*+T*+G*+G |
| 159 | A37164H | GTGCAGTGTGTGGATGTGA | +G*+T*+G*C*A*G*T*G*T*G*T*G*G*A*T*G*+T*+G*+A |
| 160 | A37165H | TTAGCATGCTCTCATATTT | +T*+T*+A*G*C*A*T*G*C*T*C*T*C*A*T*A*+T*+T*+T |
| 161 | A37166H | CTTAGCATGCTCTCATATT | +C*+T*+T*A*G*C*A*T*G*C*T*C*T*C*A*T*+A*+T*+T |
| 162 | A37167H | CCTTAGCATGCTCTCATAT | +C*+C*+T*T*A*G*C*A*T*G*C*T*C*T*C*A*+T*+A*+T |
| 163 | A37168HI | GGACTGAGAGTGAAAGGT | +G*+G*+A*C*T*G*A*G*A*G*T*G*A*A*A*+G*+G*+T |
| 164 | A37169HI | CAAGGACCGGCTGAGAGG | +C*+A*+A*G*G*A*C*C*G*G*C*T*G*A*G*+A*+G*+G |
| 165 | A37170HI | CCAAGGACCGGCTGAGAG | +C*+C*+A*A*G*G*A*C*C*G*G*C*T*G*A*+G*+A*+G |
| 166 | A37171HI | CCGTCATTCTACAGAAAC | +C*+C*+G*T*C*A*T*T*C*T*A*C*A*G*A*+A*+A*+C |
| 167 | A37172HI | AAGGCAGAGCCGCCACGC | +A*+A*+G*G*C*A*G*A*G*C*C*G*C*C*A*+C*+G*+C |
| 168 | A37173HI | CAAGGCAGAGCCGCCACG | +C*+A*+A*G*G*C*A*G*A*G*C*C*G*C*C*+A*+C*+G |
| 169 | A37174HI | AGTGAGGACCAAGGATGC | +A*+G*+T*G*A*G*G*A*C*C*A*A*G*G*A*+T*+G*+C |
| 170 | A37175HI | GAGTGAGGACCAAGGATG | +G*+A*+G*T*G*A*G*G*A*C*C*A*A*G*G*+A*+T*+G |
| 171 | A37176HI | TCGAGTGAGGACCAAGGA | +T*+C*+G*A*G*T*G*A*G*G*A*C*C*A*A*+G*+G*+A |
| 172 | A37177HI | AAACTCGAGTGAGGACCA | +A*+A*+A*C*T*C*G*A*G*T*G*A*G*G*A*+C*+C*+A |
| 173 | A37178HI | TAAAGCCAAGGTTAGTCC | +T*+A*+A*A*G*C*C*A*A*G*G*T*T*A*G*+T*+C*+C |
| 174 | A37179HI | GTAAAGCCAAGGTTAGTC | +G*+T*+A*A*A*G*C*C*A*A*G*G*T*T*A*+G*+T*+C |
| 175 | A37180HI | CGTAAAGCCAAGGTTAGT | +C*+G*+T*A*A*A*G*C*C*A*A*G*G*T*T*+A*+G*+T |
| 176 | A37181HI | TCGTAAAGCCAAGGTTAG | +T*+C*+G*T*A*A*A*G*C*C*A*A*G*G*T*+T*+A*+G |
| 177 | A37182HI | ACTCGAACAGGTACACTT | +A*+C*+T*C*G*A*A*C*A*G*G*T*A*C*A*+C*+T*+T |
| 178 | A37183HI | CACTCGAACAGGTACACT | +C*+A*+C*T*C*G*A*A*C*A*G*G*T*A*C*+A*+C*+T |
| 179 | A37184HI | CTCACTCGAACAGGTACA | +C*+T*+C*A*C*T*C*G*A*A*C*A*G*G*T*+A*+C*+A |
| 180 | A37185HI | TGTCCTCACTCGAACAGG | +T*+G*+T*C*C*T*C*A*C*T*C*G*A*A*C*+A*+G*+G |
| 181 | A37186HI | ACTGTCCTCACTCGAACA | +A*+C*+T*G*T*C*C*T*C*A*C*T*C*G*A*+A*+C*+A |
| 182 | A37187HI | AACTGTCCTCACTCGAAC | +A*+A*+C*T*G*T*C*C*T*C*A*C*T*C*G*+A*+A*+C |
| 183 | A37188HI | GAACTGTCCTCACTCGAA | +G*+A*+A*C*T*G*T*C*C*T*C*A*C*T*C*+G*+A*+A |
| 184 | A37189HI | AGAACTGTCCTCACTCGA | +A*+G*+A*A*C*T*G*T*C*C*T*C*A*C*T*+C*+G*+A |
| 185 | A37190HI | AAGAACTGTCCTCACTCG | +A*+A*+G*A*A*C*T*G*T*C*C*T*C*A*C*+T*+C*+G |
| 186 | A37191HI | ACGGATGGTCTGAACAGG | +A*+C*+G*G*A*T*G*G*T*C*T*G*A*A*C*+A*+G*+G |
| 187 | A37192HI | CCACGGATGGTCTGAACA | +C*+C*+A*C*G*G*A*T*G*G*T*C*T*G*A*+A*+C*+A |
| 188 | A37193HI | GGACTGTCTTAGGCTTGG | +G*+G*+A*C*T*G*T*C*T*T*A*G*G*C*T*+T*+G*+G |
| 189 | A37194HI | AAGAGGTGGCGCTGAGGC | +A*+A*+G*A*G*G*T*G*G*C*G*C*T*G*A*+G*+G*+C |
| 190 | A37195HI | GCTGCGGACACCTTGCTC | +G*+C*+T*G*C*G*G*A*C*A*C*C*T*T*G*+C*+T*+C |
| 191 | A37196HI | AGGCTGCGGACACCTTGC | +A*+G*+G*C*T*G*C*G*G*A*C*A*C*C*T*+T*+G*+C |
| 192 | A37197HI | CGAGTGTCAGGCTGCGGA | +C*+G*+A*G*T*G*T*C*A*G*G*C*T*G*C*+G*+G*+A |
| 193 | A37198HI | TGTCCGGCACAAGCGCGG | +T*+G*+T*C*C*G*G*C*A*C*A*A*G*C*G*+C*+G*+G |
| 194 | A37199HI | TGAGAGTCTTGTCCGGCA | +T*+G*+A*G*A*G*T*C*T*T*G*T*C*C*G*+G*+C*+A |
| 195 | A37200HI | GTGAGAGTCTTGTCCGGC | +G*+T*+G*A*G*A*G*T*C*T*T*G*T*C*C*+G*+G*+C |
| 196 | A37201HI | CTGTGAGAGTCTTGTCCG | +C*+T*+G*T*G*A*G*A*G*T*C*T*T*G*T*+C*+C*+G |
| 197 | A37202HI | AGGCATCCTGGAATGAAG | +A*+G*+G*C*A*T*C*C*T*G*G*A*A*T*G*+A*+A*+G |
| 198 | A37203HI | AGTGAATAGATCAGGAGG | +A*+G*+T*G*A*A*T*A*G*A*T*C*A*G*G*+A*+G*+G |
| 199 | A37204HI | ACGCAGTGAATAGATCAG | +A*+C*+G*C*A*G*T*G*A*A*T*A*G*A*T*+C*+A*+G |
| 200 | A37205HI | AACGCAGTGAATAGATCA | +A*+A*+C*G*C*A*G*T*G*A*A*T*A*G*A*+T*+C*+A |
| 201 | A37206HI | GAACGCAGTGAATAGATC | +G*+A*+A*C*G*C*A*G*T*G*A*A*T*A*G*+A*+T*+C |
| 202 | A37207HI | TGGCGAACGCAGTGAATA | +T*+G*+G*C*G*A*A*C*G*C*A*G*T*G*A*+A*+T*+A |
| 203 | A37208HI | ATGGCGAACGCAGTGAAT | +A*+T*+G*G*C*G*A*A*C*G*C*A*G*T*G*+A*+A*+T |
| 204 | A37209HI | AATGGCGAACGCAGTGAA | +A*+A*+T*G*G*C*G*A*A*C*G*C*A*G*T*+G*+A*+A |
| 205 | A37210HI | GAATGGCGAACGCAGTGA | +G*+A*+A*T*G*G*C*G*A*A*C*G*C*A*G*+T*+G*+A |
| 206 | A37211HI | CGAATGGCGAACGCAGTG | +C*+G*+A*A*T*G*G*C*G*A*A*C*G*C*A*+G*+T*+G |
| 207 | A37212HI | CGCTGACTGGAGCTCACA | +C*+G*+C*T*G*A*C*T*G*G*A*G*C*T*C*+A*+C*+A |
| 208 | A37213HI | ACGGAGAAGTCAGTAAGG | +A*+C*+G*G*A*G*A*A*G*T*C*A*G*T*A*+A*+G*+G |
| 209 | A37214HI | TAATCCTCAGGACGCAGC | +T*+A*+A*T*C*C*T*C*A*G*G*A*C*G*C*+A*+G*+C |
| 210 | A37215HI | TTTAATCCTCAGGACGCA | +T*+T*+T*A*A*T*C*C*T*C*A*G*G*A*C*+G*+C*+A |
| 211 | A37216HI | GTTATTGATTCTAGGTGA | +G*+T*+T*A*T*T*G*A*T*T*C*T*A*G*G*+T*+G*+A |
| 212 | A37217HI | AGTTATTGATTCTAGGTG | +A*+G*+T*T*A*T*T*G*A*T*T*C*T*A*G*+G*+T*+G |
| 213 | A37218HI | CCGTGCAGAGATGAAGGC | +C*+C*+G*T*G*C*A*G*A*G*A*T*G*A*A*+G*+G*+C |
| 214 | A37219HI | CTTCCTGTACAGAGTCAC | +C*+T*+T*C*C*T*G*T*A*C*A*G*A*G*T*+C*+A*+C |
| 215 | A37220HI | GCTTCCTGTACAGAGTCA | +G*+C*+T*T*C*C*T*G*T*A*C*A*G*A*G*+T*+C*+A |
| 216 | A37221HI | GGACTAGGTAAGATGAGG | +G*+G*+A*C*T*A*G*G*T*A*A*G*A*T*G*+A*+G*+G |
| 217 | A37222HI | AACTCCTCACAGTCGTGT | +A*+A*+C*T*C*C*T*C*A*C*A*G*T*C*G*+T*+G*+T |
| 218 | A37223HI | GGAGTGAGGTCTTCCAAC | +G*+G*+A*G*T*G*A*G*G*T*C*T*T*C*C*+A*+A*+C |
| 219 | A37224HI | GAAGCGCACAGAAGAAGG | +G*+A*+A*G*C*G*C*A*C*A*G*A*A*G*A*+A*+G*+G |
| 220 | A37225HI | CCAGAAGCGCACAGAAGA | +C*+C*+A*G*A*A*G*C*G*C*A*C*A*G*A*+A*+G*+A |
| 221 | A37226HI | CTTTCCAGAAGCGCACAG | +C*+T*+T*T*C*C*A*G*A*A*G*C*G*C*A*+C*+A*+G |
| 222 | A37227HI | CCGCATTACAGGACATTG | +C*+C*+G*C*A*T*T*A*C*A*G*G*A*C*A*+T*+T*+G |
| 223 | A37228HI | GAGAGACCGCATTACAGG | +G*+A*+G*A*G*A*C*C*G*C*A*T*T*A*C*+A*+G*+G |
| 224 | A37229HI | AGAGAGACCGCATTACAG | +A*+G*+A*G*A*G*A*C*C*G*C*A*T*T*A*+C*+A*+G |
| 225 | A37230HI | CAGAGAGACCGCATTACA | +C*+A*+G*A*G*A*G*A*C*C*G*C*A*T*T*+A*+C*+A |
| 226 | A37231HI | GCAGAGAGACCGCATTAC | +G*+C*+A*G*A*G*A*G*A*C*C*G*C*A*T*+T*+A*+C |
| 227 | A37232HI | CCTGGATCAAGTTAGATT | +C*+C*+T*G*G*A*T*C*A*A*G*T*T*A*G*+A*+T*+T |
| 228 | A37233HI | CTTGGCTGGTAGTGTCTA | +C*+T*+T*G*G*C*T*G*G*T*A*G*T*G*T*+C*+T*+A |
| 229 | A37234HI | GGTGTCTGCAGTTCAAGC | +G*+G*+T*G*T*C*T*G*C*A*G*T*T*C*A*+A*+G*+C |
| 230 | A37235HI | CTCCTGACTTGATCTGTG | +C*+T*+C*C*T*G*A*C*T*T*G*A*T*C*T*+G*+T*+G |
| 231 | A37236HI | GCTCCTGACTTGATCTGT | +G*+C*+T*C*C*T*G*A*C*T*T*G*A*T*C*+T*+G*+T |
| 232 | A37237HI | AACTGGAAACTTAGCTGC | +A*+A*+C*T*G*G*A*A*A*C*T*T*A*G*C*+T*+G*+C |
| 233 | A37238HI | GTGAGGAACAGAAGTCAT | +G*+T*+G*A*G*G*A*A*C*A*G*A*A*G*T*+C*+A*+T |
| 234 | A37239HI | CCTTCACCTGCGTGCCTG | +C*+C*+T*T*C*A*C*C*T*G*C*G*T*G*C*+C*+T*+G |
| 235 | A37240HI | CGCTCGCCTCCTTCACCT | +C*+G*+C*T*C*G*C*C*T*C*C*T*T*C*A*+C*+C*+T |
| 236 | A37241HI | GCTGACAAGCGCTCGCCT | +G*+C*+T*G*A*C*A*A*G*C*G*C*T*C*G*+C*+C*+T |
| 237 | A37242HI | CCAACTCCTAGTGCCAAC | +C*+C*+A*A*C*T*C*C*T*A*G*T*G*C*C*+A*+A*+C |
| 238 | A37243HI | CATGTGACCAGGACCAAC | +C*+A*+T*G*T*G*A*C*C*A*G*G*A*C*C*+A*+A*+C |
| 239 | A37244HI | CTCAAGGAGCAACCAGCT | +C*+T*+C*A*A*G*G*A*G*C*A*A*C*C*A*+G*+C*+T |
| 240 | A37245HI | TGACTCAAGGAGCAACCA | +T*+G*+A*C*T*C*A*A*G*G*A*G*C*A*A*+C*+C*+A |
| 241 | A37246HI | ATGACTCAAGGAGCAACC | +A*+T*+G*A*C*T*C*A*A*G*G*A*G*C*A*+A*+C*+C |
| 242 | A37247HI | GCGGTGGTGATGACTCAA | +G*+C*+G*G*T*G*G*T*G*A*T*G*A*C*T*+C*+A*+A |
| 243 | A37248HI | CAGCGAGGTGACACAGAG | +C*+A*+G*C*G*A*G*G*T*G*A*C*A*C*A*+G*+A*+G |
| 244 | A37249HI | CACTCAGTTCCGTCTCAG | +C*+A*+C*T*C*A*G*T*T*C*C*G*T*C*T*+C*+A*+G |
| 245 | A37250HI | TCACTCAGTTCCGTCTCA | +T*+C*+A*C*T*C*A*G*T*T*C*C*G*T*C*+T*+C*+A |
| 246 | A37251HI | TGTCACTCAGTTCCGTCT | +T*+G*+T*C*A*C*T*C*A*G*T*T*C*C*G*+T*+C*+T |
| 247 | A37252HI | CTGTCACTCAGTTCCGTC | +C*+T*+G*T*C*A*C*T*C*A*G*T*T*C*C*+G*+T*+C |
| 248 | A37253HI | CCGAGATGCCATGCAACG | +C*+C*+G*A*G*A*T*G*C*C*A*T*G*C*A*+A*+C*+G |
| 249 | A37254HI | CCAACACAGGCGCTTAAG | +C*+C*+A*A*C*A*C*A*G*G*C*G*C*T*T*+A*+A*+G |
| 250 | A37255HI | CACCAACACAGGCGCTTA | +C*+A*+C*C*A*A*C*A*C*A*G*G*C*G*C*+T*+T*+A |
| 251 | A37256HI | TGAAACATATGCCTGCCA | +T*+G*+A*A*A*C*A*T*A*T*G*C*C*T*G*+C*+C*+A |
| 252 | A37257HI | GACCTCCTGAAACATATG | +G*+A*+C*C*T*C*C*T*G*A*A*A*C*A*T*+A*+T*+G |
| 253 | A37258HI | TCGGATGTGGACAGACAC | +T*+C*+G*G*A*T*G*T*G*G*A*C*A*G*A*+C*+A*+C |
| 254 | A37259HI | TGACTCGGATGTGGACAG | +T*+G*+A*C*T*C*G*G*A*T*G*T*G*G*A*+C*+A*+G |
| 255 | A37260HI | TTGACTCGGATGTGGACA | +T*+T*+G*A*C*T*C*G*G*A*T*G*T*G*G*+A*+C*+A |
| 256 | A37261HI | ATGCTTCAGAGACGAGAT | +A*+T*+G*C*T*T*C*A*G*A*G*A*C*G*A*+G*+A*+T |
| 257 | A37262HI | GACTAGAGCTCACAGCAA | +G*+A*+C*T*A*G*A*G*C*T*C*A*C*A*G*+C*+A*+A |
| 258 | A37263HI | TTTCAGGACAAGCTCGGA | +T*+T*+T*C*A*G*G*A*C*A*A*G*C*T*C*+G*+G*+A |
| 259 | A37264HI | CCTACTAAGAGCCTTCAC | +C*+C*+T*A*C*T*A*A*G*A*G*C*C*T*T*+C*+A*+C |
| 260 | A37265HI | CCTTCTACGTGAGGCTGC | +C*+C*+T*T*C*T*A*C*G*T*G*A*G*G*C*+T*+G*+C |
| 261 | A37266HI | TCCTTCTACGTGAGGCTG | +T*+C*+C*T*T*C*T*A*C*G*T*G*A*G*G*+C*+T*+G |
| 262 | A37267HI | CCTCTTCCTTCTACGTGA | +C*+C*+T*C*T*T*C*C*T*T*C*T*A*C*G*+T*+G*+A |
| 263 | A37268HI | GGAATGTCATTGAGAAGT | +G*+G*+A*A*T*G*T*C*A*T*T*G*A*G*A*+A*+G*+T |
| 264 | A37269HI | CAGAGATGCCGGTCACCA | +C*+A*+G*A*G*A*T*G*C*C*G*G*T*C*A*+C*+C*+A |
| 265 | A37270HI | CTAGAGGACAGAGATGCC | +C*+T*+A*G*A*G*G*A*C*A*G*A*G*A*T*+G*+C*+C |
| 266 | A37271HI | AGCCGTGCTCCTAGGTGG | +A*+G*+C*C*G*T*G*C*T*C*C*T*A*G*G*+T*+G*+G |
| 267 | A37272HI | AGTGGATCATGCAGGAAA | +A*+G*+T*G*G*A*T*C*A*T*G*C*A*G*G*+A*+A*+A |
| 268 | A37273HI | CCGCAGGCAGGCACATAT | +C*+C*+G*C*A*G*G*C*A*G*G*C*A*C*A*+T*+A*+T |
| 269 | A37274HI | GTCTCCAATGTAAGATAA | +G*+T*+C*T*C*C*A*A*T*G*T*A*A*G*A*+T*+A*+A |
| 270 | A37275HI | TCTACAGAAACACGCAGCC | +T*+C*+T*A*C*A*G*A*A*A*C*A*C*G*C*A*+G*+C*+C |
| 271 | A37276HI | CCGTCATTCTACAGAAACA | +C*+C*+G*T*C*A*T*T*C*T*A*C*A*G*A*A*+A*+C*+A |
| 272 | A37277HI | AAGGCAGAGCCGCCACGCA | +A*+A*+G*G*C*A*G*A*G*C*C*G*C*C*A*C*+G*+C*+A |
| 273 | A37278HI | TCGTAAAGCCAAGGTTAGT | +T*+C*+G*T*A*A*A*G*C*C*A*A*G*G*T*T*+A*+G*+T |
| 274 | A37279HI | CTCATCTAAACTTTGACGT | +C*+T*+C*A*T*C*T*A*A*A*C*T*T*T*G*A*+C*+G*+T |
| 275 | A37280HI | CACCAGCTCATCTAAACTT | +C*+A*+C*C*A*G*C*T*C*A*T*C*T*A*A*A*+C*+T*+T |
| 276 | A37281HI | CTCACTCGAACAGGTACAC | +C*+T*+C*A*C*T*C*G*A*A*C*A*G*G*T*A*+C*+A*+C |
| 277 | A37282HI | AGAACTGTCCTCACTCGAA | +A*+G*+A*A*C*T*G*T*C*C*T*C*A*C*T*C*+G*+A*+A |
| 278 | A37283HI | AAGAACTGTCCTCACTCGA | +A*+A*+G*A*A*C*T*G*T*C*C*T*C*A*C*T*+C*+G*+A |
| 279 | A37284HI | GAAGAACTGTCCTCACTCG | +G*+A*+A*G*A*A*C*T*G*T*C*C*T*C*A*C*+T*+C*+G |
| 280 | A37285HI | CACGGATGGTCTGAACAGG | +C*+A*+C*G*G*A*T*G*G*T*C*T*G*A*A*C*+A*+G*+G |
| 281 | A37286HI | GGACTGTCTTAGGCTTGGC | +G*+G*+A*C*T*G*T*C*T*T*A*G*G*C*T*T*+G*+G*+C |
| 282 | A37287HI | AAGGATGCTGCCTCAGGTG | +A*+A*+G*G*A*T*G*C*T*G*C*C*T*C*A*G*+G*+T*+G |
| 283 | A37288HI | CTGACAAGGATGCTGCCTC | +C*+T*+G*A*C*A*A*G*G*A*T*G*C*T*G*C*+C*+T*+C |
| 284 | A37289HI | AAGAGGTGGCGCTGAGGCA | +A*+A*+G*A*G*G*T*G*G*C*G*C*T*G*A*G*+G*+C*+A |
| 285 | A37290HI | GAATCTCCAGTTCTGAGTC | +G*+A*+A*T*C*T*C*C*A*G*T*T*C*T*G*A*+G*+T*+C |
| 286 | A37291HI | AGAATCTCCAGTTCTGAGT | +A*+G*+A*A*T*C*T*C*C*A*G*T*T*C*T*G*+A*+G*+T |
| 287 | A37292HI | GTGAGAGTCTTGTCCGGCA | +G*+T*+G*A*G*A*G*T*C*T*T*G*T*C*C*G*+G*+C*+A |
| 288 | A37293HI | TGTGAGAGTCTTGTCCGGC | +T*+G*+T*G*A*G*A*G*T*C*T*T*G*T*C*C*+G*+G*+C |
| 289 | A37294HI | CCTGTGAGAGTCTTGTCCG | +C*+C*+T*G*T*G*A*G*A*G*T*C*T*T*G*T*+C*+C*+G |
| 290 | A37295HI | TGCCACCTGTGAGAGTCTT | +T*+G*+C*C*A*C*C*T*G*T*G*A*G*A*G*T*+C*+T*+T |
| 291 | A37296HI | AGGCATCCTGGAATGAAGA | +A*+G*+G*C*A*T*C*C*T*G*G*A*A*T*G*A*+A*+G*+A |
| 292 | A37297HI | GAGGCATCCTGGAATGAAG | +G*+A*+G*G*C*A*T*C*C*T*G*G*A*A*T*G*+A*+A*+G |
| 293 | A37298HI | AACGCAGTGAATAGATCAG | +A*+A*+C*G*C*A*G*T*G*A*A*T*A*G*A*T*+C*+A*+G |
| 294 | A37299HI | CGAATGGCGAACGCAGTGA | +C*+G*+A*A*T*G*G*C*G*A*A*C*G*C*A*G*+T*+G*+A |
| 295 | A37300HI | CTCTTATTTATGCTCCTGC | +C*+T*+C*T*T*A*T*T*T*A*T*G*C*T*C*C*+T*+G*+C |
| 296 | A37301HI | AGCAGAGGCTCTTATTTAT | +A*+G*+C*A*G*A*G*G*C*T*C*T*T*A*T*T*+T*+A*+T |
| 297 | A37302HI | CACGGAGAAGTCAGTAAGG | +C*+A*+C*G*G*A*G*A*A*G*T*C*A*G*T*A*+A*+G*+G |
| 298 | A37303HI | AAGCACACGGAGAAGTCAG | +A*+A*+G*C*A*C*A*C*G*G*A*G*A*A*G*T*+C*+A*+G |
| 299 | A37304HI | TAATCCTCAGGACGCAGCC | +T*+A*+A*T*C*C*T*C*A*G*G*A*C*G*C*A*+G*+C*+C |
| 300 | A37305HI | GTCATGGAGGCCAGATGCA | +G*+T*+C*A*T*G*G*A*G*G*C*C*A*G*A*T*+G*+C*+A |
| 301 | A37306HI | TGTCATGGAGGCCAGATGC | +T*+G*+T*C*A*T*G*G*A*G*G*C*C*A*G*A*+T*+G*+C |
| 302 | A37307HI | AGTTATTGATTCTAGGTGA | +A*+G*+T*T*A*T*T*G*A*T*T*C*T*A*G*G*+T*+G*+A |
| 303 | A37308HI | GCTCCATCCTGCACGTCCA | +G*+C*+T*C*C*A*T*C*C*T*G*C*A*C*G*T*+C*+C*+A |
| 304 | A37309HI | CCGTGCAGAGATGAAGGCA | +C*+C*+G*T*G*C*A*G*A*G*A*T*G*A*A*G*+G*+C*+A |
| 305 | A37310HI | GCTTCCTGTACAGAGTCAC | +G*+C*+T*T*C*C*T*G*T*A*C*A*G*A*G*T*+C*+A*+C |
| 306 | A37311HI | CAACTCCTCACAGTCGTGT | +C*+A*+A*C*T*C*C*T*C*A*C*A*G*T*C*G*+T*+G*+T |
| 307 | A37312HI | GTCTTCCAACTCCTCACAG | +G*+T*+C*T*T*C*C*A*A*C*T*C*C*T*C*A*+C*+A*+G |
| 308 | A37313HI | AGAAGCGCACAGAAGAAGG | +A*+G*+A*A*G*C*G*C*A*C*A*G*A*A*G*A*+A*+G*+G |
| 309 | A37314HI | CCAGAAGCGCACAGAAGAA | +C*+C*+A*G*A*A*G*C*G*C*A*C*A*G*A*A*+G*+A*+A |
| 310 | A37315HI | TTCCAGAAGCGCACAGAAG | +T*+T*+C*C*A*G*A*A*G*C*G*C*A*C*A*G*+A*+A*+G |
| 311 | A37316HI | TTTCCAGAAGCGCACAGAA | +T*+T*+T*C*C*A*G*A*A*G*C*G*C*A*C*A*+G*+A*+A |
| 312 | A37317HI | CTTTCCAGAAGCGCACAGA | +C*+T*+T*T*C*C*A*G*A*A*G*C*G*C*A*C*+A*+G*+A |
| 313 | A37318HI | CATTACAGGACATTGCTTT | +C*+A*+T*T*A*C*A*G*G*A*C*A*T*T*G*C*+T*+T*+T |
| 314 | A37319HI | CAGAGAGACCGCATTACAG | +C*+A*+G*A*G*A*G*A*C*C*G*C*A*T*T*A*+C*+A*+G |
| 315 | A37320HI | CGCAGAGAGACCGCATTAC | +C*+G*+C*A*G*A*G*A*G*A*C*C*G*C*A*T*+T*+A*+C |
| 316 | A37321HI | CCTGGATCAAGTTAGATTT | +C*+C*+T*G*G*A*T*C*A*A*G*T*T*A*G*A*+T*+T*+T |
| 317 | A37322HI | AAGGTGAATATTCAGAAGT | +A*+A*+G*G*T*G*A*A*T*A*T*T*C*A*G*A*+A*+G*+T |
| 318 | A37323HI | AGGAAGGTGAATATTCAGA | +A*+G*+G*A*A*G*G*T*G*A*A*T*A*T*T*C*+A*+G*+A |
| 319 | A37324HI | TCTACTAGGAAGGTGAATA | +T*+C*+T*A*C*T*A*G*G*A*A*G*G*T*G*A*+A*+T*+A |
| 320 | A37325HI | CCTTGGCTGGTAGTGTCTA | +C*+C*+T*T*G*G*C*T*G*G*T*A*G*T*G*T*+C*+T*+A |
| 321 | A37326HI | TGCCTCCACCAACTGGCTG | +T*+G*+C*C*T*C*C*A*C*C*A*A*C*T*G*G*+C*+T*+G |
| 322 | A37327HI | CTGCCTCCACCAACTGGCT | +C*+T*+G*C*C*T*C*C*A*C*C*A*A*C*T*G*+G*+C*+T |
| 323 | A37328HI | CCTGACTTGATCTGTGCTG | +C*+C*+T*G*A*C*T*T*G*A*T*C*T*G*T*G*+C*+T*+G |
| 324 | A37329HI | CTCCTGACTTGATCTGTGC | +C*+T*+C*C*T*G*A*C*T*T*G*A*T*C*T*G*+T*+G*+C |
| 325 | A37330HI | GCTCCTGACTTGATCTGTG | +G*+C*+T*C*C*T*G*A*C*T*T*G*A*T*C*T*+G*+T*+G |
| 326 | A37331HI | TGCTCCTGACTTGATCTGT | +T*+G*+C*T*C*C*T*G*A*C*T*T*G*A*T*C*+T*+G*+T |
| 327 | A37332HI | TTTGCACACATTGGTGGAG | +T*+T*+T*G*C*A*C*A*C*A*T*T*G*G*T*G*+G*+A*+G |
| 328 | A37333HI | ATTTGCACACATTGGTGGA | +A*+T*+T*T*G*C*A*C*A*C*A*T*T*G*G*T*+G*+G*+A |
| 329 | A37334HI | CTGCTATTTATTTGCACAC | +C*+T*+G*C*T*A*T*T*T*A*T*T*T*G*C*A*+C*+A*+C |
| 330 | A37335HI | GGAAACTTAGCTGCTATTT | +G*+G*+A*A*A*C*T*T*A*G*C*T*G*C*T*A*+T*+T*+T |
| 331 | A37336HI | ATCTGTGCATTGTTCTTGT | +A*+T*+C*T*G*T*G*C*A*T*T*G*T*T*C*T*+T*+G*+T |
| 332 | A37337HI | CATCTGTGCATTGTTCTTG | +C*+A*+T*C*T*G*T*G*C*A*T*T*G*T*T*C*+T*+T*+G |
| 333 | A37338HI | CGACAGGACAATGGCCGCT | +C*+G*+A*C*A*G*G*A*C*A*A*T*G*G*C*C*+G*+C*+T |
| 334 | A37339HI | TGGCTAGGAAGTGCTAAGG | +T*+G*+G*C*T*A*G*G*A*A*G*T*G*C*T*A*+A*+G*+G |
| 335 | A37340HI | AGGAACAGAAGTCATCACG | +A*+G*+G*A*A*C*A*G*A*A*G*T*C*A*T*C*+A*+C*+G |
| 336 | A37341HI | GTGAGGAACAGAAGTCATC | +G*+T*+G*A*G*G*A*A*C*A*G*A*A*G*T*C*+A*+T*+C |
| 337 | A37342HI | GGTGAGGAACAGAAGTCAT | +G*+G*+T*G*A*G*G*A*A*C*A*G*A*A*G*T*+C*+A*+T |
| 338 | A37343HI | GCAGGTGAGGAACAGAAGT | +G*+C*+A*G*G*T*G*A*G*G*A*A*C*A*G*A*+A*+G*+T |
| 339 | A37344HI | ACAGGCAGGTGAGGAACAG | +A*+C*+A*G*G*C*A*G*G*T*G*A*G*G*A*A*+C*+A*+G |
| 340 | A37345HI | GGACTCGGCACAGAGCAGG | +G*+G*+A*C*T*C*G*G*C*A*C*A*G*A*G*C*+A*+G*+G |
| 341 | A37346HI | GCCTGAATGGAGGAAGATG | +G*+C*+C*T*G*A*A*T*G*G*A*G*G*A*A*G*+A*+T*+G |
| 342 | A37347HI | GCTCGCCTCCTTCACCTGC | +G*+C*+T*C*G*C*C*T*C*C*T*T*C*A*C*C*+T*+G*+C |
| 343 | A37348HI | CGCTCGCCTCCTTCACCTG | +C*+G*+C*T*C*G*C*C*T*C*C*T*T*C*A*C*+C*+T*+G |
| 344 | A37349HI | GGTTTGAAGTGACCTTGAG | +G*+G*+T*T*T*G*A*A*G*T*G*A*C*C*T*T*+G*+A*+G |
| 345 | A37350HI | CCTAGTGCCAACCTCACTG | +C*+C*+T*A*G*T*G*C*C*A*A*C*C*T*C*A*+C*+T*+G |
| 346 | A37351HI | TGACCAGGACCAACTCCTA | +T*+G*+A*C*C*A*G*G*A*C*C*A*A*C*T*C*+C*+T*+A |
| 347 | A37352HI | AGCAACCAGCTCAGAGGAG | +A*+G*+C*A*A*C*C*A*G*C*T*C*A*G*A*G*+G*+A*+G |
| 348 | A37353HI | TGACTCAAGGAGCAACCAG | +T*+G*+A*C*T*C*A*A*G*G*A*G*C*A*A*C*+C*+A*+G |
| 349 | A37354HI | ATGACTCAAGGAGCAACCA | +A*+T*+G*A*C*T*C*A*A*G*G*A*G*C*A*A*+C*+C*+A |
| 350 | A37355HI | GGCGGTGGTGATGACTCAA | +G*+G*+C*G*G*T*G*G*T*G*A*T*G*A*C*T*+C*+A*+A |
| 351 | A37356HI | GGTGACACAGAGACCAGGC | +G*+G*+T*G*A*C*A*C*A*G*A*G*A*C*C*A*+G*+G*+C |
| 352 | A37357HI | CGAGGTGACACAGAGACCA | +C*+G*+A*G*G*T*G*A*C*A*C*A*G*A*G*A*+C*+C*+A |
| 353 | A37358HI | CAGCGAGGTGACACAGAGA | +C*+A*+G*C*G*A*G*G*T*G*A*C*A*C*A*G*+A*+G*+A |
| 354 | A37359HI | CCAGCGAGGTGACACAGAG | +C*+C*+A*G*C*G*A*G*G*T*G*A*C*A*C*A*+G*+A*+G |
| 355 | A37360HI | CACTCAGTTCCGTCTCAGG | +C*+A*+C*T*C*A*G*T*T*C*C*G*T*C*T*C*+A*+G*+G |
| 356 | A37361HI | TCACTCAGTTCCGTCTCAG | +T*+C*+A*C*T*C*A*G*T*T*C*C*G*T*C*T*+C*+A*+G |
| 357 | A37362HI | TGTCACTCAGTTCCGTCTC | +T*+G*+T*C*A*C*T*C*A*G*T*T*C*C*G*T*+C*+T*+C |
| 358 | A37363HI | CTGTCACTCAGTTCCGTCT | +C*+T*+G*T*C*A*C*T*C*A*G*T*T*C*C*G*+T*+C*+T |
| 359 | A37364HI | GCACCAACACAGGCGCTTA | +G*+C*+A*C*C*A*A*C*A*C*A*G*G*C*G*C*+T*+T*+A |
| 360 | A37365HI | GGAGGCACCAACACAGGCG | +G*+G*+A*G*G*C*A*C*C*A*A*C*A*C*A*G*+G*+C*+G |
| 361 | A37366HI | CTGAAACATATGCCTGCCA | +C*+T*+G*A*A*A*C*A*T*A*T*G*C*C*T*G*+C*+C*+A |
| 362 | A37367HI | CCTGAAACATATGCCTGCC | +C*+C*+T*G*A*A*A*C*A*T*A*T*G*C*C*T*+G*+C*+C |
| 363 | A37368HI | GACCTCCTGAAACATATGC | +G*+A*+C*C*T*C*C*T*G*A*A*A*C*A*T*A*+T*+G*+C |
| 364 | A37369HI | ACAAGGACCTCCTGAAACA | +A*+C*+A*A*G*G*A*C*C*T*C*C*T*G*A*A*+A*+C*+A |
| 365 | A37370HI | CCAAGACAAGGACCTCCTG | +C*+C*+A*A*G*A*C*A*A*G*G*A*C*C*T*C*+C*+T*+G |
| 366 | A37371HI | ACTCGGATGTGGACAGACA | +A*+C*+T*C*G*G*A*T*G*T*G*G*A*C*A*G*+A*+C*+A |
| 367 | A37372HI | TGACTCGGATGTGGACAGA | +T*+G*+A*C*T*C*G*G*A*T*G*T*G*G*A*C*+A*+G*+A |
| 368 | A37373HI | ATTGACTCGGATGTGGACA | +A*+T*+T*G*A*C*T*C*G*G*A*T*G*T*G*G*+A*+C*+A |
| 369 | A37374HI | CATTGACTCGGATGTGGAC | +C*+A*+T*T*G*A*C*T*C*G*G*A*T*G*T*G*+G*+A*+C |
| 370 | A37375HI | ATGCTTCAGAGACGAGATG | +A*+T*+G*C*T*T*C*A*G*A*G*A*C*G*A*G*+A*+T*+G |
| 371 | A37376HI | GATGCTTCAGAGACGAGAT | +G*+A*+T*G*C*T*T*C*A*G*A*G*A*C*G*A*+G*+A*+T |
| 372 | A37377HI | GCAAAGATGCTTCAGAGAC | +G*+C*+A*A*A*G*A*T*G*C*T*T*C*A*G*A*+G*+A*+C |
| 373 | A37378HI | TAGAGCTCACAGCAAAGAT | +T*+A*+G*A*G*C*T*C*A*C*A*G*C*A*A*A*+G*+A*+T |
| 374 | A37379HI | CTAGAGCTCACAGCAAAGA | +C*+T*+A*G*A*G*C*T*C*A*C*A*G*C*A*A*+A*+G*+A |
| 375 | A37380HI | ACTAGAGCTCACAGCAAAG | +A*+C*+T*A*G*A*G*C*T*C*A*C*A*G*C*A*+A*+A*+G |
| 376 | A37381HI | GACTAGAGCTCACAGCAAA | +G*+A*+C*T*A*G*A*G*C*T*C*A*C*A*G*C*+A*+A*+A |
| 377 | A37382HI | GGACTAGAGCTCACAGCAA | +G*+G*+A*C*T*A*G*A*G*C*T*C*A*C*A*G*+C*+A*+A |
| 378 | A37383HI | CCTGTCTGCACTGCTCTGG | +C*+C*+T*G*T*C*T*G*C*A*C*T*G*C*T*C*+T*+G*+G |
| 379 | A37384HI | CCTGATTTCCTACTAAGAG | +C*+C*+T*G*A*T*T*T*C*C*T*A*C*T*A*A*+G*+A*+G |
| 380 | A37385HI | TTCCTTCTACGTGAGGCTG | +T*+T*+C*C*T*T*C*T*A*C*G*T*G*A*G*G*+C*+T*+G |
| 381 | A37386HI | GCCTCTTCCTTCTACGTGA | +G*+C*+C*T*C*T*T*C*C*T*T*C*T*A*C*G*+T*+G*+A |
| 382 | A37387HI | TGCAGAGCCTCTTCCTTCT | +T*+G*+C*A*G*A*G*C*C*T*C*T*T*C*C*T*+T*+C*+T |
| 383 | A37388HI | TCATTGAGAAGTCTCTGCT | +T*+C*+A*T*T*G*A*G*A*A*G*T*C*T*C*T*+G*+C*+T |
| 384 | A37389HI | GTCATTGAGAAGTCTCTGC | +G*+T*+C*A*T*T*G*A*G*A*A*G*T*C*T*C*+T*+G*+C |
| 385 | A37390HI | AGCTGGAATGTCATTGAGA | +A*+G*+C*T*G*G*A*A*T*G*T*C*A*T*T*G*+A*+G*+A |
| 386 | A37391HI | CTAGAGGACAGAGATGCCG | +C*+T*+A*G*A*G*G*A*C*A*G*A*G*A*T*G*+C*+C*+G |
| 387 | A37392HI | GCTAGAGGACAGAGATGCC | +G*+C*+T*A*G*A*G*G*A*C*A*G*A*G*A*T*+G*+C*+C |
| 388 | A37393HI | CAGAGCTAGAGGACAGAGA | +C*+A*+G*A*G*C*T*A*G*A*G*G*A*C*A*G*+A*+G*+A |
| 389 | A37394HI | CAGTGGATCATGCAGGAAA | +C*+A*+G*T*G*G*A*T*C*A*T*G*C*A*G*G*+A*+A*+A |
| 390 | A37395HI | TATAATAGAATGTGAGTCC | +T*+A*+T*A*A*T*A*G*A*A*T*G*T*G*A*G*+T*+C*+C |
| 391 | A37396HI | CCGCAGGCAGGCACATATG | +C*+C*+G*C*A*G*G*C*A*G*G*C*A*C*A*T*+A*+T*+G |
| 392 | A37397HI | GATAAGAAATGACCAAGCC | +G*+A*+T*A*A*G*A*A*A*T*G*A*C*C*A*A*+G*+C*+C |
| 393 | A37398HI | TGTAAGATAAGAAATGACC | +T*+G*+T*A*A*G*A*T*A*A*G*A*A*A*T*G*+A*+C*+C |
| 394 | A37399HI | CTCCTGTCTCCAATGTAAG | +C*+T*+C*C*T*G*T*C*T*C*C*A*A*T*G*T*+A*+A*+G |
| 395 | A37400HI | CTCTCCTGTCTCCAATGTA | +C*+T*+C*T*C*C*T*G*T*C*T*C*C*A*A*T*+G*+T*+A |
| 396 | A37401HI | GCTCTCCTGTCTCCAATGT | +G*+C*+T*C*T*C*C*T*G*T*C*T*C*C*A*A*+T*+G*+T |
| 397 | A37402HI | AGCTCTCCTGTCTCCAATG | +A*+G*+C*T*C*T*C*C*T*G*T*C*T*C*C*A*+A*+T*+G |
| 398 | A37403HI | AAGCTCTCCTGTCTCCAAT | +A*+A*+G*C*T*C*T*C*C*T*G*T*C*T*C*C*+A*+A*+T |
| 399 | A37404HI | CAAGCTCTCCTGTCTCCAA | +C*+A*+A*G*C*T*C*T*C*C*T*G*T*C*T*C*+C*+A*+A |
| 400 | A37405HI | TCAAGCTCTCCTGTCTCCA | +T*+C*+A*A*G*C*T*C*T*C*C*T*G*T*C*T*+C*+C*+A |
| 401 | A37406HI | TCTTGCAGATTTAGGATTC | +T*+C*+T*T*G*C*A*G*A*T*T*T*A*G*G*A*+T*+T*+C |
| 402 | A37407HI | TTCTTGCAGATTTAGGATT | +T*+T*+C*T*T*G*C*A*G*A*T*T*T*A*G*G*+A*+T*+T |
| 403 | A37408HI | TGGCATTCTTGCAGATTTA | +T*+G*+G*C*A*T*T*C*T*T*G*C*A*G*A*T*+T*+T*+A |
| 404 | A37409HI | CTGGCATTCTTGCAGATTT | +C*+T*+G*G*C*A*T*T*C*T*T*G*C*A*G*A*+T*+T*+T |
| 405 | A37410HI | CCTGGCATTCTTGCAGATT | +C*+C*+T*G*G*C*A*T*T*C*T*T*G*C*A*G*+A*+T*+T |
| 406 | Neg1 | | +C*+G*+T*T*T*A*G*G*C*T*A*T*G*T*A*+C*+T*+T |
| 407 | R01011 | | +G*+A*+T*C*A*T*T*C*G*C*G*G*A*C*+A*+A*+C |
| 408 | R1019 | | +G*+A*+C*T*C*G*T*T*A*A*A*C*C*G*+A*+T*+A |

Table 1: List of antisense oligonucleotides hybridizing with human PD-1 for example of SEQ ID NO.1; Neg1, R01011 and R1019 are antisense oligonucleotides representing negative controls which are not hybridizing with PD-1 of SEQ ID NO.1. Some of these antisense oligonucleotides do not only hybridize with exons of human PD-1 pre-mRNA (H), some of these only with introns of human PD-1 pre-mRNA (HI) and some of these with exons of human and of mouse PD-1 pre-mRNA (HM), respectively.

The antisense oligonucleotides of the present invention hybridize for example with exons and/or introns of the pre-mRNA of human PD-1 of SEQ ID NO.1. Such antisense oligonucleotides are called PD-1 antisense oligonucleotides. In some embodiments, the oligonucleotides hybridize within a hybridizing active area which is one or more region(s) on the PD-1 pre-mRNA, e.g., of SEQ ID NO.1, where hybridization with an oligonucleotide highly likely results in a potent knockdown of the PD-1 expression. In the present invention surprisingly several hybridizing active areas were identified for example selected from hybridizing active areas shown in the following Table 2 (in bold) and examples of antisense oligonucleotides of the present invention hybridizing with these areas:

| Hybridizing active area (in bold) | First position on >NG_012110.1:5001-14026 Homo sapiens programmed cell death 1 (PDCD1), RefSeqGene on chromosome 2 |
|---|---|
| 0-299 | |
| A37001H (SEQ ID NO.2) | 38 |
| A37002H (SEQ ID NO.2) | 38 |
| A37003H (SEQ ID NO.2) | 38 |
| A37091H (SEQ ID NO.86) | 9 |
| A37092H (SEQ ID NO.87) | 29 |
| A37093H (SEQ ID NO.88) | 59 |
| A37094H (SEQ ID NO.89) | 125 |
| A37168HI (SEQ ID NO. 163) | 285 |

| | |
|---|---|
| 300-599 | |
| A37023HI (SEQ ID NO.21) | 588 |
| A37169HI (SEQ ID NO. 164) | 473 |
| A37170HI (SEQ ID NO. 165) | 474 |
| A37171HI (SEQ ID NO. 166) | 500 |
| A37172HI (SEQ ID NO. 167) | 560 |
| A37173HI (SEQ ID NO. 168) | 561 |
| A37174HI (SEQ ID NO. 169) | 584 |
| A37175HI (SEQ ID NO. 170) | 585 |
| A37176HI (SEQ ID NO.171) | 587 |
| A37177HI (SEQ ID NO. 172) | 591 |
| A37275HI (SEQ ID NO.270) | 492 |
| A37276HI (SEQ ID NO.271) | 499 |
| A37277HI (SEQ ID NO.272) | 559 |

| | |
|---|---|
| 600-899 | |
| A37024HI (SEQ ID NO.22) | 642 |
| A37025HI (SEQ ID NO.23) | 714 |
| A37026HI (SEQ ID NO.24) | 851 |
| A37178HI (SEQ ID NO.173) | 636 |
| A37179HI (SEQ ID NO. 174) | 637 |
| A37180HI (SEQ ID NO.175) | 638 |
| A37181HI (SEQ ID NO. 176) | 639 |
| A37182HI (SEQ ID NO. 177) | 702 |
| A37183HI (SEQ ID NO. 178) | 703 |
| A37184HI (SEQ ID NO. 179) | 705 |
| A37185HI (SEQ ID NO. 180) | 709 |
| A37186HI (SEQ ID NO.181) | 711 |
| A37187HI (SEQ ID NO. 182) | 712 |
| A37188HI (SEQ ID NO. 183) | 713 |
| A37189HI (SEQ ID NO. 184) | 714 |
| A37190HI (SEQ ID NO. 185) | 715 |
| A37191HI (SEQ ID NO. 186) | 809 |
| A37192HI (SEQ ID NO. 187) | 811 |
| A37278HI (SEQ ID NO.273) | 638 |
| A37279HI (SEQ ID NO.274) | 656 |
| A37280HI (SEQ ID NO.275) | 662 |
| A37281HI (SEQ ID NO.276) | 704 |
| A37282HI (SEQ ID NO.277) | 713 |
| A37283HI (SEQ ID NO.278) | 714 |
| A37284HI (SEQ ID NO.279) | 715 |
| A37285HI (SEQ ID NO.280) | 809 |

| | |
|---|---|
| 900-1199 | |
| A37193HI (SEQ ID NO. 188) | 1021 |
| A37286HI (SEQ ID NO.281) | 1020 |

| 1200–1499 | |
|---|---|
| A37027HI (SEQ ID NO.25) | 1458 |
| A37194HI (SEQ ID NO. 189) | 1256 |
| A37287HI (SEQ ID NO.282) | 1218 |
| A37288HI (SEQ ID NO.283) | 1223 |
| A37289HI (SEQ ID NO.284) | 1255 |
| A37290HI (SEQ ID NO.285) | 1298 |
| A37291HI (SEQ ID NO.286) | 1299 |
| 1500–1799 | |
| A37028HI (SEQ ID NO.26) | 1558 |
| A37029HI (SEQ ID NO.26) | 1558 |
| A37030HI (SEQ ID NO.27) | 1567 |
| A37031HI (SEQ ID NO.28) | 1733 |
| A37032HI (SEQ ID NO.29) | 1742 |
| A37195HI (SEQ ID NO. 190) | 1517 |
| A37196HI (SEQ ID NO. 191) | 1519 |
| A37197HI (SEQ ID NO. 192) | 1527 |
| A37198HI (SEQ ID NO. 193) | 1557 |
| A37199HI (SEQ ID NO. 194) | 1566 |
| A37200HI (SEQ ID NO. 195) | 1567 |
| A37201HI (SEQ ID NO. 196) | 1569 |
| A37202HI (SEQ ID NO. 197) | 1703 |
| A37203HI (SEQ ID NO. 198) | 1727 |
| A37204HI (SEQ ID NO. 199) | 1731 |
| A37205HI (SEQ ID NO.200) | 1732 |
| A37206HI (SEQ ID NO.201) | 1733 |
| A37207HI (SEQ ID NO.202) | 1737 |
| A37208HI (SEQ ID NO.203) | 1738 |
| A37209HI (SEQ ID NO.204) | 1739 |
| A37210HI (SEQ ID NO.205) | 1740 |
| A37211HI (SEQ ID NO.206) | 1741 |
| A37292HI (SEQ ID NO.287) | 1566 |
| A37293HI (SEQ ID NO.288) | 1567 |
| A37294HI (SEQ ID NO.289) | 1569 |
| A37295HI (SEQ ID NO.290) | 1574 |
| A37296HI (SEQ ID NO.291) | 1702 |
| A37297HI (SEQ ID NO.292) | 1703 |
| A37298HI (SEQ ID NO.293) | 1731 |
| A37299HI (SEQ ID NO.294) | 1740 |
| 1800–2099 | |
| A37033HI (SEQ ID NO. 30) | 1934 |
| A37212HI (SEQ ID NO.207) | 1897 |
| A37213HI (SEQ ID NO.208) | 1955 |
| A37214HI (SEQ ID NO.209) | 2010 |
| A37215HI (SEQ ID NO.210) | 2012 |
| A37300HI (SEQ ID NO.295) | 1855 |
| A37301HI (SEQ ID NO.296) | 1863 |
| A37302HI (SEQ ID NO.297) | 1955 |
| A37303HI (SEQ ID NO.298) | 1960 |
| A37304HI (SEQ ID NO.299) | 2009 |
| 2100–2399 | |
| A37305HI (SEQ ID NO. 300) | 2342 |
| A37306HI (SEQ ID NO.301) | 2343 |
| 2400–2699 | |
| A37034HI (SEQ ID NO.31) | 2542 |
| A37035HI (SEQ ID NO. 32) | 2543 |
| A37216HI (SEQ ID NO.211) | 2465 |
| A37217HI (SEQ ID NO.212) | 2466 |
| A37307HI (SEQ ID NO. 302) | 2465 |
| A37308HI (SEQ ID NO.303) | 2594 |
| 2700–2999 | |
| A37036HI (SEQ ID NO.33) | 2946 |
| A37218HI (SEQ ID NO.213) | 2840 |
| A37219HI (SEQ ID NO.214) | 2858 |
| A37220HI (SEQ ID NO.215) | 2859 |
| A37221HI (SEQ ID NO.216) | 2912 |
| A37309HI (SEQ ID NO. 304) | 2839 |
| A37310HI (SEQ ID NO.305) | 2858 |
| 3000–3299 | |
| A37037HI (SEQ ID NO. 34) | 3168 |
| A37038HI (SEQ ID NO.35) | 3264 |
| A37222HI (SEQ ID NO.217) | 3049 |
| A37223HI (SEQ ID NO.218) | 3064 |
| A37224HI (SEQ ID NO.219) | 3228 |
| A37225HI (SEQ ID NO.220) | 3231 |
| A37226HI (SEQ ID NO.221) | 3235 |
| A37227HI (SEQ ID NO.222) | 3253 |
| A37228HI (SEQ ID NO.223) | 3259 |
| A37229HI (SEQ ID NO.224) | 3260 |
| A37230HI (SEQ ID NO.225) | 3261 |
| A37231HI (SEQ ID NO.226) | 3262 |
| A37311HI (SEQ ID NO. 306) | 3049 |
| A37312HI (SEQ ID NO.307) | 3055 |
| A37313HI (SEQ ID NO. 308) | 3228 |
| A37314HI (SEQ ID NO. 309) | 3230 |
| A37315HI (SEQ ID NO.310) | 3232 |
| A37316HI (SEQ ID NO.311) | 3233 |
| A37317HI (SEQ ID NO.312) | 3234 |
| A37318HI (SEQ ID NO.313) | 3249 |
| A37319HI (SEQ ID NO.314) | 3260 |
| A37320HI (SEQ ID NO.315) | 3262 |
| 3300–3599 | |
| A37039HI (SEQ ID NO. 36) | 3387 |
| A37232HI (SEQ ID NO.227) | 3448 |
| A37233HI (SEQ ID NO.228) | 3504 |
| A37321HI (SEQ ID NO.316) | 3447 |
| A37322HI (SEQ ID NO.317) | 3480 |
| A37323HI (SEQ ID NO.318) | 3483 |
| A37324HI (SEQ ID NO.319) | 3489 |
| A37325HI (SEQ ID NO.320) | 3504 |
| 3600–3899 | |
| A37040HI (SEQ ID NO.37) | 3873 |
| A37234HI (SEQ ID NO.229) | 3662 |
| A37235HI (SEQ ID NO.230) | 3881 |
| A37236HI (SEQ ID NO.231) | 3882 |
| A37326HI (SEQ ID NO.321) | 3702 |
| A37327HI (SEQ ID NO.322) | 3703 |
| A37328HI (SEQ ID NO.323) | 3878 |
| A37329HI (SEQ ID NO. 324) | 3880 |
| A37330HI (SEQ ID NO.325) | 3881 |
| A37331HI (SEQ ID NO.326) | 3882 |
| 3900–4199 | |
| A37041HI (SEQ ID NO. 38) | 4000 |
| A37237HI (SEQ ID NO.232) | 3931 |
| A37238HI (SEQ ID NO.233) | 4093 |
| A37332HI (SEQ ID NO.327) | 3906 |
| A37333HI (SEQ ID NO.328) | 3907 |
| A37334HI (SEQ ID NO.329) | 3916 |
| A37335HI (SEQ ID NO.330) | 3926 |
| A37336HI (SEQ ID NO.331) | 3949 |
| A37337HI (SEQ ID NO.332) | 3950 |
| A37338HI (SEQ ID NO.333) | 3998 |
| A37339HI (SEQ ID NO.334) | 4046 |
| A37340HI (SEQ ID NO.335) | 4089 |
| A37341HI (SEQ ID NO.336) | 4092 |
| A37342HI (SEQ ID NO.337) | 4093 |
| A37343HI (SEQ ID NO.338) | 4096 |
| A37344HI (SEQ ID NO.339) | 4100 |
| 4200–4499 | |
| A37042HI (SEQ ID NO. 39) | 4481 |
| A37345HI (SEQ ID NO. 340) | 4241 |
| A37346HI (SEQ ID NO.341) | 4351 |
| 4500–4799 | |
| A37043HI (SEQ ID NO.40) | 4645 |
| A37044HI (SEQ ID NO.40) | 4645 |
| A37045HI (SEQ ID NO.41) | 4762 |
| A37239HI (SEQ ID NO.234) | 4626 |
| A37240HI (SEQ ID NO.235) | 4635 |
| A37241HI (SEQ ID NO.236) | 4644 |
| A37242HI (SEQ ID NO.237) | 4758 |
| A37243HI (SEQ ID NO.238) | 4771 |
| A37347HI (SEQ ID NO. 342) | 4633 |
| A37348HI (SEQ ID NO. 343) | 4634 |
| A37349HI (SEQ ID NO. 344) | 4706 |
| A37350HI (SEQ ID NO. 345) | 4751 |
| A37351HI (SEQ ID NO. 346) | 4766 |
| 4800–5099 | |
| A37244HI (SEQ ID NO.239) | 4878 |
| A37245HI (SEQ ID NO.240) | 4881 |
| A37246HI (SEQ ID NO.241) | 4882 |
| A37247HI (SEQ ID NO.242) | 4891 |
| A37248HI (SEQ ID NO.243) | 4973 |
| A37249HI (SEQ ID NO.244) | 5008 |
| A37250HI (SEQ ID NO.245) | 5009 |
| A37251HI (SEQ ID NO.246) | 5011 |
| 5100–5399 | |
| A37252HI (SEQ ID NO.247) | 5012 |
| A37352HI (SEQ ID NO. 347) | 4870 |
| A37353HI (SEQ ID NO. 348) | 4880 |
| A37354HI (SEQ ID NO. 349) | 4881 |
| A37355HI (SEQ ID NO.350) | 4891 |
| A37356HI (SEQ ID NO.351) | 4966 |
| A37357HI (SEQ ID NO.352) | 4969 |
| A37358HI (SEQ ID NO.353) | 4972 |
| A37359HI (SEQ ID NO.354) | 4973 |
| A37360HI (SEQ ID NO.355) | 5007 |
| A37361HI (SEQ ID NO.356) | 5008 |
| A37362HI (SEQ ID NO.357) | 5010 |
| A37363HI (SEQ ID NO.358) | 5011 |
| A37046HI (SEQ ID NO.42) | 5116 |
| A37253HI (SEQ ID NO.248) | 5116 |
| A37254HI (SEQ ID NO.249) | 5142 |
| A37255HI (SEQ ID NO.250) | 5144 |
| A37256HI (SEQ ID NO.251) | 5225 |
| A37257HI (SEQ ID NO.252) | 5232 |
| A37258HI (SEQ ID NO.253) | 5281 |
| A37259HI (SEQ ID NO.254) | 5285 |
| A37260HI (SEQ ID NO.255) | 5286 |
| A37261HI (SEQ ID NO.256) | 5310 |
| A37262HI (SEQ ID NO.257) | 5330 |
| A37364HI (SEQ ID NO.359) | 5144 |
| A37365HI (SEQ ID NO. 360) | 5148 |
| A37366HI (SEQ ID NO.361) | 5225 |
| A37367HI (SEQ ID NO. 362) | 5226 |
| A37368HI (SEQ ID NO.363) | 5231 |
| A37369HI (SEQ ID NO. 364) | 5236 |
| A37370HI (SEQ ID NO.365) | 5241 |
| A37371HI (SEQ ID NO. 366) | 5282 |
| A37372HI (SEQ ID NO.367) | 5284 |
| A37373HI (SEQ ID NO. 368) | 5286 |
| A37374HI (SEQ ID NO. 369) | 5287 |
| A37375HI (SEQ ID NO.370) | 5309 |
| A37376HI (SEQ ID NO.371) | 5310 |
| A37377HI (SEQ ID NO.372) | 5315 |
| A37378HI (SEQ ID NO.373) | 5326 |
| A37379HI (SEQ ID NO.374) | 5327 |
| A37380HI (SEQ ID NO.375) | 5328 |
| A37381HI (SEQ ID NO.376) | 5329 |
| A37382HI (SEQ ID NO. 377) | 5330 |
| 5400–5699 | |
| A37263HI (SEQ ID NO.258) | 5427 |
| A37264HI (SEQ ID NO.259) | 5662 |
| A37383HI (SEQ ID NO.378) | 5553 |
| A37384HI (SEQ ID NO.379) | 5669 |
| 5700-5999 | |
| A37004H (SEQ ID NO. 3) | 5970 |
| A37005H (SEQ ID NO.4) | 5971 |
| A37006H (SEQ ID NO. 5) | 5975 |
| A37047HI (SEQ ID NO.43) | 5724 |
| A37053H (SEQ ID NO. 48) | 5970 |
| A37054H (SEQ ID NO.49) | 5973 |
| A37055H (SEQ ID NO. 50) | 5993 |
| A37056H (SEQ ID NO.51) | 5994 |
| A37095H (SEQ ID NO.90) | 5971 |
| A37096H (SEQ ID NO.91) | 5972 |
| A37097H (SEQ ID NO.92) | 5993 |
| A37098H (SEQ ID NO.93) | 5994 |
| A37265HI (SEQ ID NO.260) | 5724 |
| A37266HI (SEQ ID NO.261) | 5725 |
| A37267HI (SEQ ID NO.262) | 5730 |
| A37268HI (SEQ ID NO.263) | 5800 |
| A37385HI (SEQ ID NO. 380) | 5725 |
| A37386HI (SEQ ID NO.381) | 5730 |
| A37387HI (SEQ ID NO. 382) | 5736 |
| A37388HI (SEQ ID NO.383) | 5793 |
| A37389HI (SEQ ID NO. 384) | 5794 |
| A37390HI (SEQ ID NO.385) | 5803 |
| 6000–6299 | |
| A37007H (SEQ ID NO.6) | 6024 |
| A37008H (SEQ ID NO.7) | 6027 |
| A37009H (SEQ ID NO.8) | 6035 |
| A37010H (SEQ ID NO.9) | 6038 |
| A37011H (SEQ ID NO. 10) | 6043 |
| A37012HM (SEQ ID NO.11) | 6068 |
| A37013H (SEQ ID NO. 12) | 6126 |
| A37014H (SEQ ID NO.13) | 6203 |
| A37015HM (SEQ ID NO. 14) | 6240 |
| A37057H (SEQ ID NO. 52) | 6021 |
| A37058H (SEQ ID NO.53) | 6023 |
| A37059H (SEQ ID NO. 54) | 6027 |
| A37060H (SEQ ID NO.55) | 6035 |
| A37061H (SEQ ID NO. 56) | 6036 |
| A37062H (SEQ ID NO.57) | 6037 |
| A37063H (SEQ ID NO. 58) | 6043 |
| A37064H (SEQ ID NO. 59) | 6044 |
| A37065H (SEQ ID NO.60) | 6066 |
| A37066H (SEQ ID NO.61) | 6067 |
| A37067H (SEQ ID NO.62) | 6127 |
| A37068H (SEQ ID NO.63) | 6133 |
| A37069H (SEQ ID NO. 64) | 6134 |
| A37070H (SEQ ID NO.65) | 6158 |
| A37071H (SEQ ID NO.66) | 6160 |
| A37072H (SEQ ID NO.67) | 6203 |
| A37073H (SEQ ID NO.68) | 6239 |
| A37074H (SEQ ID NO.69) | 6240 |
| A37075H (SEQ ID NO.70) | 6243 |
| A37076H (SEQ ID NO.71) | 6249 |
| A37077H (SEQ ID NO.72) | 6250 |
| A37099H (SEQ ID NO. 94) | 6002 |
| A37100H (SEQ ID NO.95) | 6006 |
| A37101H (SEQ ID NO.96) | 6008 |
| A37102H (SEQ ID NO.97) | 6010 |
| A37103H (SEQ ID NO.98) | 6011 |
| A37104H (SEQ ID NO.99) | 6021 |
| A37105H (SEQ ID NO. 100) | 6022 |
| A37106H (SEQ ID NO.101) | 6026 |
| A37107H (SEQ ID NO. 102) | 6027 |
| A37108H (SEQ ID NO. 103) | 6029 |
| A37109H (SEQ ID NO. 104) | 6035 |
| A37110H (SEQ ID NO. 105) | 6036 |
| A37111H (SEQ ID NO. 106) | 6042 |
| A37112H (SEQ ID NO. 107) | 6043 |
| A37113H (SEQ ID NO. 108) | 6044 |
| A37114H (SEQ ID NO. 109) | 6065 |
| A37115H (SEQ ID NO.110) | 6066 |
| A37116H (SEQ ID NO.111) | 6067 |
| A37117H (SEQ ID NO.112) | 6126 |
| A37118H (SEQ ID NO.113) | 6130 |
| A37119H (SEQ ID NO. 114) | 6133 |
| A37120H (SEQ ID NO.115) | 6134 |
| A37121H (SEQ ID NO. 116) | 6158 |
| A37122H (SEQ ID NO.117) | 6166 |
| A37123H (SEQ ID NO.118) | 6203 |
| A37124H (SEQ ID NO. 119) | 6239 |
| A37125H (SEQ ID NO. 120) | 6240 |
| A37126H (SEQ ID NO. 121) | 6248 |

| | |
|---|---|
| 6300–6599 | |
| A37269HI (SEQ ID NO.264) | 6431 |
| A37270HI (SEQ ID NO.265) | 6439 |
| A37391HI (SEQ ID NO. 386) | 6438 |
| A37392HI (SEQ ID NO.387) | 6439 |
| A37393HI (SEQ ID NO. 388) | 6443 |

| 6600–6899 | |
|---|---|
| A37016HM (SEQ ID NO.15) | 6622 |
| A37048HI (SEQ ID NO.44) | 6870 |
| A37078H (SEQ ID NO.73) | 6621 |
| A37079H (SEQ ID NO. 74) | 6622 |
| A37127H (SEQ ID NO. 122) | 6621 |
| A37128H (SEQ ID NO. 123) | 6622 |
| A37129H (SEQ ID NO. 124) | 6656 |
| A37271HI (SEQ ID NO.266) | 6867 |
| 6900–7199 | |
| A37272HI (SEQ ID NO.267) | 6973 |
| A37394HI (SEQ ID NO. 389) | 6973 |
| A37395HI (SEQ ID NO. 390) | 7050 |
| 7200–7499 | |
| A37273HI (SEQ ID NO.268) | 7251 |
| A37274HI (SEQ ID NO.269) | 7335 |
| A37396HI (SEQ ID NO.391) | 7250 |
| A37397HI (SEQ ID NO. 392) | 7321 |
| A37398HI (SEQ ID NO.393) | 7326 |
| A37399HI (SEQ ID NO. 394) | 7339 |
| A37400HI (SEQ ID NO.395) | 7341 |
| A37401HI (SEQ ID NO. 396) | 7342 |
| A37402HI (SEQ ID NO.397) | 7343 |
| A37403HI (SEQ ID NO. 398) | 7344 |
| A37404HI (SEQ ID NO. 399) | 7345 |
| A37405HI (SEQ ID NO. 400) | 7346 |
| A37406HI (SEQ ID NO.401) | 7379 |
| A37407HI (SEQ ID NO.402) | 7380 |
| A37408HI (SEQ ID NO.403) | 7385 |
| A37409HI (SEQ ID NO. 404) | 7386 |
| A37410HI (SEQ ID NO.405) | 7387 |
| 7500–7799 | |
| A37017H (SEQ ID NO. 16) | 7711 |
| A37018H (SEQ ID NO.17) | 7738 |
| A37049HI (SEQ ID NO.45) | 7513 |
| A37050HI (SEQ ID NO.45) | 7513 |
| A37051HI (SEQ ID NO. 46) | 7514 |
| A37052HI (SEQ ID NO.47) | 7518 |
| A37080H (SEQ ID NO.75) | 7624 |
| A37081H (SEQ ID NO.76) | 7634 |
| A37082H (SEQ ID NO. 77) | 7635 |
| A37083H (SEQ ID NO.78) | 7636 |
| A37084H (SEQ ID NO.79) | 7667 |
| A37085H (SEQ ID NO.80) | 7709 |
| A37086H (SEQ ID NO.81) | 7737 |
| A37130H (SEQ ID NO. 125) | 7624 |
| A37131H (SEQ ID NO. 126) | 7625 |
| A37132H (SEQ ID NO. 127) | 7630 |
| A37133H (SEQ ID NO. 128) | 7633 |
| A37134H (SEQ ID NO. 129) | 7634 |
| A37135H (SEQ ID NO. 130) | 7635 |
| A37136H (SEQ ID NO.131) | 7662 |
| A37137H (SEQ ID NO. 132) | 7663 |
| A37138H (SEQ ID NO.133) | 7709 |
| A37139H (SEQ ID NO. 134) | 7719 |
| A37140H (SEQ ID NO.135) | 7722 |
| A37141H (SEQ ID NO. 136) | 7723 |
| A37142H (SEQ ID NO.137) | 7724 |
| A37143H (SEQ ID NO. 138) | 7734 |
| 7800–8099 | |
| A37019H (SEQ ID NO. 18) | 7939 |
| A37087H (SEQ ID NO.82) | 7845 |
| A37088H (SEQ ID NO.83) | 8022 |
| A37089H (SEQ ID NO. 84) | 8023 |
| A37144H (SEQ ID NO. 139) | 7843 |
| A37145H (SEQ ID NO. 140) | 7844 |
| A37146H (SEQ ID NO.141) | 7859 |
| A37147H (SEQ ID NO. 142) | 7899 |
| A37148H (SEQ ID NO. 143) | 7903 |
| A37149H (SEQ ID NO. 144) | 8016 |
| A37150H (SEQ ID NO. 145) | 8018 |
| A37151H (SEQ ID NO. 146) | 8022 |
| 8100–8399 | |
| A37020H (SEQ ID NO. 19) | 8195 |
| A37152H (SEQ ID NO. 147) | 8100 |
| A37153H (SEQ ID NO. 148) | 8121 |
| A37154H (SEQ ID NO. 149) | 8144 |
| A37155H (SEQ ID NO. 150) | 8253 |
| A37156H (SEQ ID NO.151) | 8254 |
| A37157H (SEQ ID NO. 152) | 8257 |
| A37158H (SEQ ID NO.153) | 8392 |
| 8400–8699 | |
| A37159H (SEQ ID NO. 154) | 8460 |
| A37160H (SEQ ID NO.155) | 8511 |
| A37161H (SEQ ID NO. 156) | 8575 |
| A37162H (SEQ ID NO.157) | 8577 |
| 8700–8999 | |
| A37021H (SEQ ID NO.20) | 8823 |
| A37022H (SEQ ID NO.20) | 8823 |
| A37163H (SEQ ID NO. 158) | 8870 |
| A37164H (SEQ ID NO. 159) | 8877 |
| 9000–9299 | |
| A37090H (SEQ ID NO.85) | 9008 |
| A37165H (SEQ ID NO. 160) | 9005 |
| A37166H (SEQ ID NO.161) | 9006 |
| A37167H (SEQ ID NO. 162) | 9007 |

In some embodiments, the antisense oligonucleotide of the present invention inhibits for example at least about 25 % to 99 %, 30 % to 95 %, 35 % to 90 %, 40 % to 85 %, 45 % to 80 %, 50 % to 75 %, 55 % to 70 %, e.g., 30 %, 35 %, 40 %, 45 %, 50%, 55%, 60%, 65%, 70%, 75%, 80%, 85%, 90%, 92%, 94%, 95%, 96%, 97%, 98%, 99% or 100% of PD-1 expression such as the, e.g., human, rat or murine PD-1 expression for example in comparison to an untreated cell, tissue, organ, subject. Thus, the antisense oligonucleotides of the present invention are for example immunosuppression-reverting oligonucleotides which inhibit and revert immunosuppression, respectively, for example in a cell, tissue, organ, or a subject. The antisense oligonucleotide of the present invention inhibits the expression of PD-1 at a nanomolar or micromolar concentration for example in a concentration of 0.1, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900 or 950 nM, or 1, 10 or 100 µM.

The antisense oligonucleotide of the present invention is for example used in a concentration of 1, 3, 5, 9, 10, 15, 27, 30, 40, 50, 75, 82, 100, 250, 300, 500, or 740 nM, or 1, 2.2, 3, 5, 6.6 or 10 µM.

The present invention refers for example to a pharmaceutical composition comprising an antisense oligonucleotide of the present invention and a pharmaceutically acceptable carrier, excipient and/or dilutant. In some embodiments, the pharmaceutical composition further comprises a chemotherapeutic, another disease specific active agent such as another oligonucleotide of the present invention or a different oligonucleotide hybridizing with the PD-1 mRNA or a different target, an antibody, a HERA fusion protein, a ligand trap, a Fab fragment, a nanobody, a BiTe, a small molecule or a combination thereof which is for example effective in preventing and/or treating a malignant tumor, a benign tumor and/or an infectious disease. The pharmaceutical composition is likewise used in cell therapy. It is added to an isolated immune cell for example in the ex vivo step of a cell therapy.

The oligonucleotide or the pharmaceutical composition of the present invention is for example for use in a method of preventing and/or treating a disorder such as a malignant tumor and/or a benign tumor. In some embodiments, the use of the oligonucleotide or the pharmaceutical composition of the present invention in a method of preventing and/or treating a disorder is combined with radiotherapy. The radiotherapy may be further combined with a chemotherapy (e.g., platinum, gemcitabine). The disorder is for example characterized by a PD-1 imbalance, i.e., the PD-1 level is increased in comparison to the level in a normal, healthy cell, tissue, organ or subject. The PD-1 level is for example increased by an increased PD-1 expression, function and/or activity. The PD-1 level can be measured by any standard method known to a person skilled in the art such as immunohistochemistry, western blot, quantitative real time PCR or QuantiGene assay.

An antisense oligonucleotide or a pharmaceutical composition of the present invention is administered locally or systemically for example orally, sublingually, nasally, subcutaneously, intravenously, intraperitoneally, intramuscularly, intratumoral, intrathecal, transdermal, and/or rectal. The oligonucleotide is administered alone or in combination with another antisense oligonucleotide of the present invention and optionally in combination with another compound such as another oligonucleotide, an antibody, a HERA fusion protein, a ligand trap, a Fab fragment, a nanobody, a BiTe, a small molecule and/or a chemotherapeutic (e.g., platinum, gemcitabine) and/or another disease specific agent such as a PD-1 antibody. In some embodiments, the other oligonucleotide (i.e., not being part of the present invention), the antibody, a HERA fusion protein, a ligand trap, a Fab fragment, a nanobody, a BiTe, and/or the small molecule are effective in preventing and/or treating an autoimmune disorder, an immune disorder, diabetes, artheriosclerosis, a nephrological disorder and/or cancer. Alternatively or in addition, the antisense oligonucleotide is used in ex vivo treatment of an immune cell such as a T cell.

For example the antisense oligonucleotide of the present invention and a compound selected from the group consisting of a chemotherapeutic, another oligonucleotide of the present invention or a different oligonucleotide hybridizing with the PD-1 mRNA or a different target, an antibody, a HERA fusion protein, a ligand trap, a Fab fragment, a nanobody, a BiTe, a small molecule or a combination thereof are for use in cell therapy, wherein the antisense oligonucleotide is administered to an isolated immune cell in an ex vivo step of a cell therapy and the compound is administered to a subject, for example suffering from a disease caused by PD-1 imbalance, receiving cell therapy. Alternatively or in addition, the immune cell donor is under treatment with a compound selected from the group consisting of a chemotherapeutic, another disease specific active agent such as another oligonucleotide of the present invention or a different oligonucleotide hybridizing with the PD-1 mRNA or a different target, an antibody, a HERA fusion protein, a ligand trap, a Fab fragment, a nanobody, a BiTe, a small molecule or a combination thereof.

An antisense oligonucleotide or a pharmaceutical composition of the present invention is used for example in a method of preventing and/or treating a solid tumor or a hematologic tumor. Examples of cancers preventable and/or treatable by use of the oligonucleotide or pharmaceutical composition of the present invention are breast cancer, lung cancer, malignant melanoma, lymphoma, skin cancer, bone cancer, prostate cancer, liver cancer, brain cancer, cancer of the larynx, gall bladder, pancreas, testicular, rectum, parathyroid, thyroid, adrenal, neural tissue, head and neck, colon, stomach, bronchi, kidneys, basal cell carcinoma, squamous cell carcinoma, metastatic skin carcinoma, osteo sarcoma, Ewing’s sarcoma, reticulum cell sarcoma, liposarcoma, myeloma, giant cell tumor, small-cell lung tumor, islet cell tumor, primary brain tumor, meningioma, acute and chronic lymphocytic and granulocytic tumors, acute and chronic myeloid leukemia, hairy-cell tumor, adenoma, hyperplasia, medullary carcinoma, intestinal ganglioneuromas, Wilm’s tumor, seminoma, ovarian tumor, leiomyomater tumor, cervical dysplasia, retinoblastoma, soft tissue sarcoma, malignant carcinoid, topical skin lesion, rhabdomyosarcoma, Kaposi’s sarcoma, osteogenic sarcoma, malignant hypercalcemia, renal cell tumor, polycythermia vera, adenocarcinoma, anaplastic astrocytoma, glioblastoma multiforma, leukemia, or epidermoid carcinoma.

Further examples of diseases preventable and/or treatable by use of the oligonucleotide or pharmaceutical composition of the present invention other than cancer are for example an infectious disease.

The infectious disease is for example selected from the group consisting of a Hepatitis B infection, a Hepatitis A infection, a Cytomegalovirus infection, an Epstein-Barr-Virus infection, an Adenovirus infection or a combination thereof.

All these diseases are for example caused or influenced by a PD-1 imbalance.

For example two or more antisense oligonucleotides of the present invention are administered together, at the same time point for example in a pharmaceutical composition or separately, or on staggered intervals. In other embodiments, one or more oligonucleotides of the present invention are administered together with another compound such as another oligonucleotide (i.e., not being part of the present invention), an antibody, a HERA fusion protein, a ligand trap, a Fab fragment, a nanobody, a BiTe, a small molecule and/or a chemotherapeutic, at the same time point for example in a pharmaceutical composition or separately, or on staggered intervals. In some embodiments of these combinations, the antisense oligonucleotide of the present invention inhibits the expression, function and/or activity of an immune suppressive factor and the other oligonucleotide (i.e., not being part of the present invention), the antibody, a HERA fusion protein, a ligand trap, a Fab fragment, a nanobody, a BiTe and/or small molecule inhibits (antagonist) or stimulates (agonist) the same and/or another immune suppressive factor and/or an immune stimulatory factor. The immune suppressive factor is for example selected from the group consisting of IDO1, IDO2, CTLA-4, PD-1, PD-L1, LAG-3, 2B4, CD304, PQR-prot, PERK, FOXP3, GMCSF, INFg, TNFa, TGFb, IL-1, IL-2, IL-6, IL-10, IL-12, IL-17, IL-9, STAT3, IL-6 receptor, VISTA, A2AR, CD39, CD73, STAT3, TDO2, TIM-3, TIGIT, TGF-beta, BTLA, MICA, NKG2A, KIR, CD160, Chop, Xbp1 and a combination thereof. The immune stimulatory factor is for example selected from the group consisting of 4-1BB, Ox40, KIR, GITR, CD27, 2B4 and a combination thereof or encodes a protein that affects expansion and/or survival of the immune cell selected from the group consisting of BID, BIM, BAD, NOXA, PUMA, BAX, BAK, BOK, BCL-rambo, BCL-Xs, Hrk, Blk, BMf, p53 and a combination thereof.

The immune suppressive factor is a factor whose expression, function and/or activity is for example increased in a cell, tissue, organ or subject. The immune stimulatory factor is a factor whose level is increased or decreased in a cell, tissue, organ or subject depending on the cell, tissue, organ or subject and its individual conditions.

An antibody in combination with the antisense oligonucleotide or the pharmaceutical composition of the present invention is for example an anti-PD-1 antibody (e.g., Cemiplimab, CT-011, Nivolumab, Pembrolizumab), an anti-PD-L1 antibody (e.g., Atezolizumab, Avelumab, Durvalumab), a CTLA-4 antibody (e.g., Ipilimumab) or a bispecific antibody. A small molecule in combination with the antisense oligonucleotide or the pharmaceutical composition of the present invention are for example Epacadostat, Vemurafenib, or a tyrosine kinase inhibitor.

A subject of the present invention is for example a human being for example of any genetic background; non-human animal comprises mammalian such as horse, cattle, pig, lamb, cat, dog, guinea pig, hamster etc.; fish such as trout, salmon, zander; bird such as goose, duck, ostrich etc. for example of any genetic background.

Moreover, the antisense oligonucleotide of the present invention is used in a cell therapy such as a T cell therapy. The antisense oligonucleotide is highly advantageous for example over an antibody, siRNA and sdRNA, respectively. The antisense oligonucleotide is administered in vivo as well as ex vivo without any delivery system such as a delivery agent or electroporation. Consequently, it does not have any negative effects on cell viability for example resulting in negative side effects of a cell therapy.

The present invention further relates to a method for reducing expression, function and/or activity of PD-1 in an isolated cell such as an immune cell in preparation for cell therapy. The method comprises the steps of incubating the isolated cell such as an immune cell comprising the PD-1 RNA with an antisense oligonucleotide without use of a transfection means such as gymnotic transfection. The antisense oligonucleotide is administered to the isolated cell such as an immune cell at least once in a time period of day 0 to day 21. The antisense oligonucleotide hybridizes with the PD-1 RNA and reduces the expression, function and/or activity of PD-1 up to 8 weeks from day 0 of the incubation with the antisense oligonucleotide. As the administration of the antisense oligonucleotides does not permanently block the expression, function and/or activity of PD-1, side effects are avoided which are based on permanent blocking of RNA expression, function and/or activity. Additionally, administration of an antisense oligonucleotide without transfection means significantly reduces the stress on a cell and reduces or even avoids side effects caused by other transfection means.

The isolated cell is for example an immune cell, a stem cell, a pluripotent stem cell such as an induced pluripotent stem cell, an embryonic stem cell, a skin stem cell, a cord blood stem cell, a mesenchymal stem cell, a neural stem cell or a combination thereof. The immune cell is for example selected from the group consisting of a T cell, a dendritic cell, a natural killer (NK) cell, a peripheral blood mononuclear cell (PBMC), a hematopoietic stem cell, a B cell and a combination thereof. T cells are for example genetically modified to express an antigen-specific receptor such as a chimeric antigen receptor or a T cell receptor. Those cells can exert their anti-tumor function by recognizing an antigen on the surface of a tumor cell via the antigen-specific receptor, which leads to activation of the T cell. The activated T cell releases cytokines and toxic molecules that lead to destruction of the tumor cell.

The PD-1 RNA is for example mRNA, pre-mRNA, lncRNA, and/or miRNA. The oligonucleotide hybridizes with a specific sequence of the PD-1 RNA and reduces the expression, function and/or activity of the PD-1 (e.g., RNA or protein) consisting of or comprising this sequence.

The cell used in the method of reducing expression of PD-1 RNA is for example isolated from a human or non-human animal. The human animal is for example a human being for example of any genetic background; non-human animal comprises mammalian such as horse, cattle, pig, lamb, cat, dog, guinea pig, hamster etc.; fish such as trout, salmon, zander; bird such as goose, duck, ostrich etc. for example of any genetic background.

The isolated cell is optionally genetically modified by a gene transfer technology including 1) transfection by (bio)chemical methods, 2) transfection by physical methods and 3) virus-mediated transduction. (Bio)chemical methods are for example calcium phosphate transfection, transfection with DEAE-dextran, or lipofection; physical methods are for example electroporation, nucleofection, microinjection, transfection by particle bombardment or transfection by ultrasound; and virus-mediated transduction uses for example adenoviruses for short-term infections with high-level transient expression, herpesviruses for long-term expression, or retroviruses or lentivirus for stable integration of DNA into the host cell genome. Following the genetic modification the cell is expanded. The genetic modification is for example permanent or transient.

The isolated cell is for example incubated with the antisense oligonucleotide of the present invention before or after the genetic modification and/or before or after the expansion of the genetically modified cell. Optionally, the isolated cell is purified, e.g., by one or more washing steps, before and/or after incubation with the antisense oligonucleotide.

The method of the present invention optionally comprises a concentrating step, wherein the isolated cell is concentrated via any concentration method of the art before and/or after the incubation with the antisense oligonucleotide. An antisense oligonucleotide is for example administered to the isolated cell again after the concentrating step.

Further, the isolated cell is for example cryopreserved when incubated with the antisense oligonucleotide, before incubation with the antisense oligonucleotide and/or after incubation with the antisense oligonucleotide, after any purification step, after any concentrating step or a combination thereof.

Isolation according to the present invention means obtaining cells from a source, e.g., immune cells from blood, stem cell from bone marrow or blood of the umbilical cord etc., and/or obtaining a subpopulation of cells from previously isolated cells or a cell population.

The method of reducing expression of PD-1 RNA optionally comprises an activation step, wherein the isolated cell is activated via any activation method of the art for example by stimulating the cell using monoclonal antibodies specific for CD3 and CD23 on the surface of T cells before and/or after the incubation with the antisense oligonucleotide of the present invention. The antisense oligonucleotide is for example administered to the isolated cell again after the activation step.

The method of reducing expression of PD-1 RNA optionally comprises an expansion step, wherein the isolated cells is expanded via any expansion method of the art for example by adding basic fibroblast growth factor (FGF2) to mesenchymal stem cells before and/or after the incubation with the oligonucleotide or by adding interleukin-2 (IL-2) and/or interleukin-15 (IL-15) to NK cells before and/or after the incubation with the oligonucleotide.

The isolated cell is incubated with the PD-1 antisense oligonucleotide for a time period (incubation period) of for example day 0 to day 21, of day 0 to day 20, of day 0 to day 19, of day 0 to day 18, of day 0 to day 17, of day 0 to day 16, of day 0 to day 15, of day 0 to day 14, of day 0 to day 13, of day 0 to day 12, of day 0 to day 11, of day 0 to day 10, of day 0 to day 9, of day 0 to day 8, of day 0 to day 7, of day 0 to day 6, of day 0 to day 5, of day 0 to day 4, of day 0 to day 3, of day 0 to day 2 or of day 0 to day 1. Day 0 is the day when the first antisense oligonucleotide is added the first time to the isolated cell. The PD-1 antisense oligonucleotide is for example added only once to the isolated cell, or every day during the time period or every second day, every third day, every fourth day, every fifth day, every sixth day, every seventh day, every eighth day, every ninth day, every tenth day of the time period or only on the first and the last day of the time period, which represent administration patterns. During the incubation period any administration pattern can be combined, e.g., the incubation period is day 0 to day 9, where the PD-1 antisense oligonucleotide is administered for five days every day and for four days every second day. After the time period the oligonucleotide is for example removed from the isolated cell. The PD-1 antisense oligonucleotide is added to the isolated cell in a nanomolar or micromolar range for example 0.1 nmol to 1000 µmol, 0.5 nmol to 900 µmol, 1 nmol to 800 µmol, 50 nmol to 700 µmol, 100 nmol to 600 µmol, 200 nmol to 500 µmol, 300 nmol to 400 µmol, 500 nmol to 300 µmol, 600 nmol to 200 µmol, 700 nmol to 100 µmol, or 800 nmol to 50 µmol.

The PD-1 antisense oligonucleotide reduces the expression of the target RNA for example for at least 10 weeks, for at least 8 weeks, for at least 6 weeks, for at least 4 weeks, or for at least 2 weeks from day 0 of the incubation period. The antisense oligonucleotide of the present invention reduces PD-1 RNA expression for example up to 3, 4, 5, 6, 7, 8, 9, 10, 11, 12, 13, 14, 15, 16, 17, 18, 19, 20, or 21 days in a cell, tissue, organ or subject after removal of the antisense oligonucleotide from the cell or up to 3, 4, 5, 6, 7, 8, 9, 10, 11, 12, 13, 14, 15, 16, 17, 18, 19, 20, or 21 days in a cell, tissue, organ or subject after addition of the antisense oligonucleotide. The reduction of the expression of the PD-1 RNA is for example independent of the incubation period with the oligonucleotide. These reduction terms of the expression of the PD-1 RNA are reached with each of the above mentioned incubation periods.

The isolated cell is for example incubated with one or more, e.g., 2, 3, 4, 5, 6, 7, 8, 9 or 10 different antisense oligonucleotides of the present invention or of the present invention in combination with any other oligonucleotide hybridizing with the same (PD-1) or a different target. The different oligonucleotides are administered to the isolated cell at the same time point for the same time period, at the same time point for different time periods, at different time points for the same period or at different time points for different time periods.

Alternatively or in addition, the PD-1 target RNA is one or more target RNAs, i.e., the same antisense oligonucleotide of the present invention for example reduces the expression of more than one target RNA, different oligonucleotides reduce the expression of different target RNAs, e.g., in parallel or subsequently having a direct and/or indirect effect on the factor of interest.

The present invention is further directed to the isolated cell obtainable by the method of reducing expression of PD-1 RNA. The isolated cell is for example for use in a method of preventing and/or treating a disease. The cell is for example isolated from a patient suffering from the disease or from a healthy subject and the isolated cell is incubated ex vivo with the antisense oligonucleotide or the pharmaceutical composition of the present invention hybridizing with the PD-1 RNA according to the method of the present invention. After incubating the isolated cell with the antisense oligonucleotide, the isolated cell is reintroduced into the patient from whom it was isolated. Alternatively, the cell isolated from a healthy subject and incubated ex vivo with the antisense oligonucleotide of the present invention hybridizing with the PD-1 RNA according to the method of reducing expression of PD-1 RNA is introduced into a patient suffering from a disease based on PD-1 imbalance. Thus, the present invention comprises allogenic cell therapy. The antisense oligonucleotide treated immune cell is for example reintroduced or introduced into the patient intravenously, intraperitoneally, intramuscularly and/or subcutaneously.

The cell such as an immune cell for use in a method of preventing and/or treating a disease comprises isolated cells from a patient, a healthy subject or a combination thereof, which have been incubated ex vivo with the antisense oligonucleotide of the present invention hybridizing with the PD-1 target RNA according to the present invention. In the method of reducing expression of PD-1 RNA either the antisense oligonucleotide and/or the pharmaceutical composition comprising such antisense oligonucleotide is used.

### EXAMPLES

The following examples illustrate different embodiments of the present invention, but the invention is not limited to these examples. The following experiments are performed on cells endogenously expressing PD-1, i.e., the cells do not represent an artificial system comprising transfected reporter constructs. Such artificial systems generally show a higher degree of inhibition and lower IC₅₀ values than endogenous systems which are closer to therapeutically relevant in vivo systems. Further, in the following experiments no transfecting agent is used, i.e., gymnotic delivery is performed. Transfecting agents are known to increase the activity of an antisense oligonucleotide which influences the IC₅₀ value (see for example Zhang et al., Gene Therapy, 2011, 18, 326-333; Stanton et al., Nucleic Acid Therapeutics, Vol. 22, No. 5, 2012). As artificial systems using a transfecting agent are hard or impossible to be translated into therapeutic approaches and no transfection formulation has been approved so far for antisense oligonucleotides, the following experiments are performed without any transfecting agent.

### Example 1: Design of Human Programmed Death Ligand 1 (PD-1) Antisense Oligonucleotides (ASOs)

For the design of ASOs with specificity for human PD-1 the PD-1 pre-mRNA sequence of SEQ ID NO.1 was used. 15, 16, 17, 18 and 19 mers were designed according to in house criteria, negl (described in WO2014154843 A1), R01011 or R01019 (both designed in house) were used as control oligonucleotides (Table 1).

### Example 2: Efficacy Screen of PD-1-Specific ASOs in Human Cancer Cell Lines

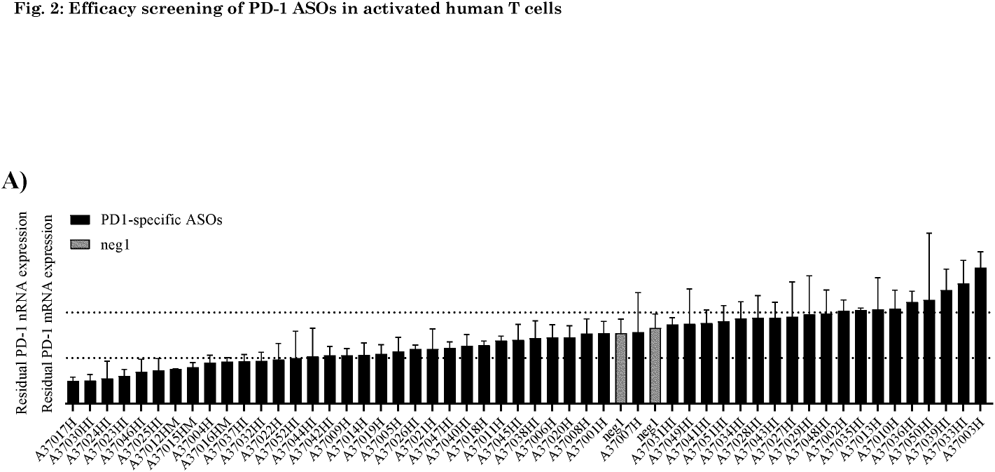

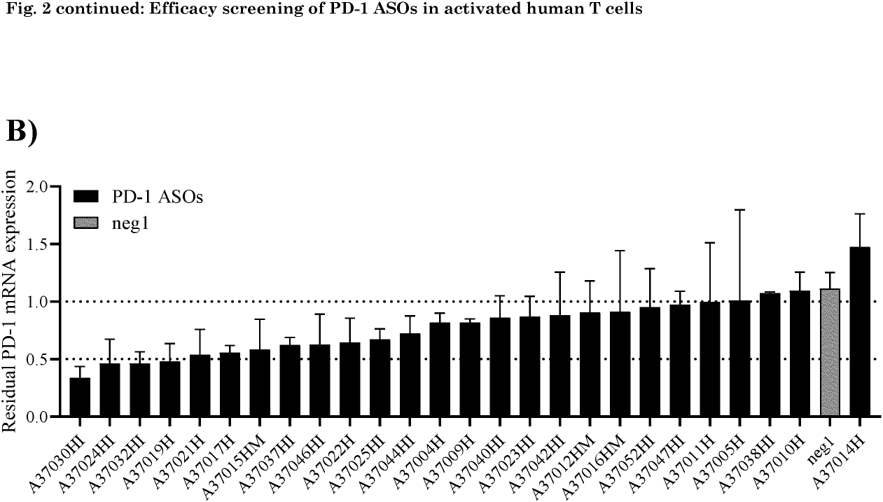

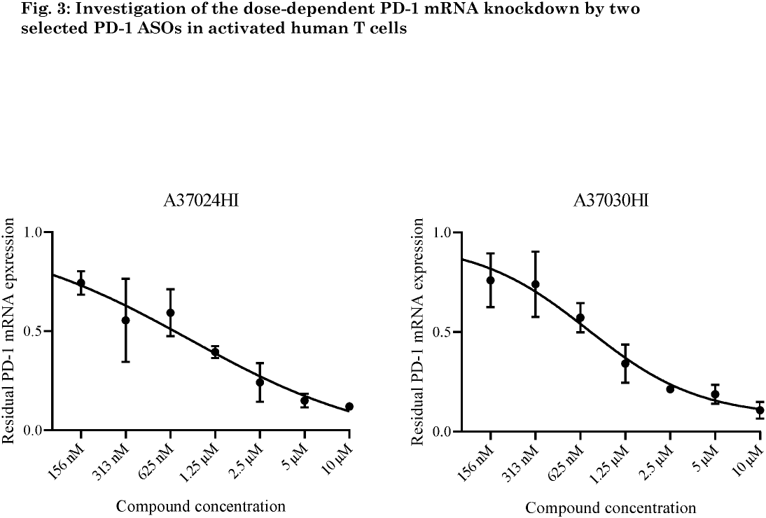

In order to investigate the knockdown efficacy of the in silico designed PD-1 ASOs, efficacy screens were performed in activated human T cells from two different donors. Therefore, T cells were isolated, activated with CD3/CD28 antibodies and were treated with the respective ASO or the control oligonucleotide neg1 at a concentration of 5 µM for three days without the addition of a transfection reagent. Cells were lyzed after the three days treatment period, PD-1 and HPRT1 mRNA expression were analyzed using the QuantiGene Singleplex assay (ThermoFisher) and the PD-1 expression values were normalized to HPRT1 values. As depicted in FIG. 2A and Table 3, treatment of activated human T cells from donor 1 with the ASOs A37017H (SEQ ID NO.16), A37030HI (SEQ ID NO.27), A37024HI (SEQ ID NO.22), A37023HI (SEQ ID NO.21), A37046HI (SEQ ID NO.42), A37025HI (SEQ ID NO.23), A37012HM (SEQ ID NO.11), A37015HM (SEQ ID NO.14), A37004H (SEQ ID NO.3), A37016HM (SEQ ID NO.15), A37037HI (SEQ ID NO.34), A37032HI (SEQ ID NO.29) and A37022H (SEQ ID NO.22) resulted in a residual PD-1 mRNA expression of <0.5. The control oligonucleotide negl had only a minimal effect on the PD-1 mRNA expression in this experiment. Selected ASOs were furthermore screened in activated human T cells from donor 2 with regard to their PD-1 knockdown efficacy. As shown in FIG. 3 and Table 4, treatment with the ASOs A37030HI (SEQ ID NO.27), A37024HI (SEQ ID NO.22), A37032HI (SEQ ID NO.29) and A37019H (SEQ ID NO.18) resulted in a residual PD-1 mRNA expression of <0.5, whereas the control oligonucleotide neg1 had no effect.

TABLE 3

List of the mean PD-1 mRNA expression values in ASO-treated activated human T cells from donor 1. PD-1 expression values were normalized for HPRT1 expression values. Residual PD-1 mRNA expression as compared to mock-treated cells is shown.

| ASO | Residual PD-1 mRNA expression (normalized for HPRT1, compared to mock-treated cells) |
|---|---|
| A37017H | 0.25 |
| A37030HI | 0.25 |
| A37024HI | 0.28 |
| A37023HI | 0.30 |
| A37046HI | 0.35 |
| A37025HI | 0.36 |
| A37012HM | 0.38 |
| A37015HM | 0.40 |
| A37004H | 0.45 |
| A37016HM | 0.46 |
| A37037HI | 0.47 |
| A37032HI | 0.47 |
| A37022H | 0.48 |
| A37052HI | 0.50 |
| A37044HI | 0.52 |
| A37042HI | 0.53 |
| A37009H | 0.53 |
| A37014H | 0.53 |
| A37019H | 0.54 |
| A37005H | 0.57 |
| A37026HI | 0.60 |
| A37021H | 0.60 |
| A37047HI | 0.61 |
| A37040HI | 0.64 |
| A37018H | 0.64 |
| A37011H | 0.69 |
| A37045HI | 0.70 |
| A37038HI | 0.72 |
| A37006H | 0.73 |
| A37020H | 0.73 |
| A37008H | 0.77 |
| A37001H | 0.77 |
| neg1 | 0.77 |
| A37007H | 0.78 |
| neg1 | 0.83 |
| A37031HI | 0.87 |
| A37049HI | 0.88 |
| A37041HI | 0.88 |
| A37051HI | 0.90 |
| A37034HI | 0.93 |
| A37028HI | 0.94 |
| A37043HI | 0.94 |
| A37027HI | 0.95 |
| A37029HI | 0.98 |
| A37048HI | 0.99 |
| A37002H | 1.02 |
| A37035HI | 1.03 |
| A37013H | 1.03 |
| A37010H | 1.04 |
| A37036HI | 1.12 |
| A37050HI | 1.14 |
| A37039HI | 1.25 |
| A37033HI | 1.32 |
| A37003H | 1.49 |

TABLE 4

List of the mean PD-1 mRNA expression values in ASO-treated activated human T cells from donor 2. PD-1 expression values were normalized for HPRT1 expression values. Residual PD-1 mRNA expression as compared to mock-treated cells is shown.

| ASO | Residual PD-1 mRNA expression (normalized for HPRT1, compared to mock-treated cells) |
|---|---|
| A37030HI | 0.34 |
| A37024HI | 0.46 |
| A37032HI | 0.46 |
| A37019H | 0.48 |
| A37021H | 0.54 |
| A37017H | 0.56 |
| A37015HM | 0.58 |
| A37037HI | 0.62 |
| A37046HI | 0.63 |
| A37022H | 0.64 |
| A37025HI | 0.67 |
| A37044HI | 0.72 |
| A37004H | 0.82 |
| A37009H | 0.82 |
| A37040HI | 0.86 |
| A37023HI | 0.87 |
| A37042HI | 0.88 |
| A37012HM | 0.91 |
| A37016HM | 0.91 |
| A37052HI | 0.95 |
| A37047HI | 0.97 |
| A37011H | 1.00 |
| A37005H | 1.01 |
| A37038HI | 1.07 |
| A37010H | 1.09 |
| negl | 1.11 |
| A37014H | 1.47 |

### Example 3: Determination of IC₅₀ Values of selected PD-1 ASOs in Activated Human T Cells

The dose-dependent knockdown of PD-1 mRNA expression by PD-1 ASOs in activated human T cells was investigated and the respective IC₅₀ values were calculated. Therefore, T cells were isolated, activated and treated for three days with the respective ASO at the following concentrations: 10 µM, 5 µM, 2.5 µM, 1.25 µM, 625 nM, 313 nM, 156 nM. After the treatment period, cells were lyzed, PD-1 and HPRT1 mRNA expression was analyzed using the QuantiGene Singleplex assay (ThermoFisher) and the PD-1 expression values were normalized to HPRT1 values. Residual PD-1 mRNA expression as compared to mock-treated cells is depicted. A dose-dependent knockdown of PD-1 mRNA (FIG. 3 and Table 5) with IC₅₀ values of 839 nM and 704 nM was observed.

TABLE 5

Dose-dependent inhibition of PD-1 mRNA expression in activated human T cells by two selected PD-1 ASOs and respective IC₅₀ values.
Inhibition (%)

| ASO | IC₅₀ (nM) | 10 µM | 51 µM | 2.5 µM | 1.25 µM | 625 nM | 313 nM | 156 nM |
|---|---|---|---|---|---|---|---|---|
| A37024HI (SEQ ID NO.22) | 839 | 88 | 85 | 76 | 61 | 41 | 45 | 26 |
| A37030HI (SEQ ID NO.27) | 704 | 89 | 81 | 79 | 66 | 43 | 26 | 24 |

### Example 4: Time-Dependency of PD-1 Knockdown in Activated Human T Cells After Treatment with Selected PD-1 ASOs

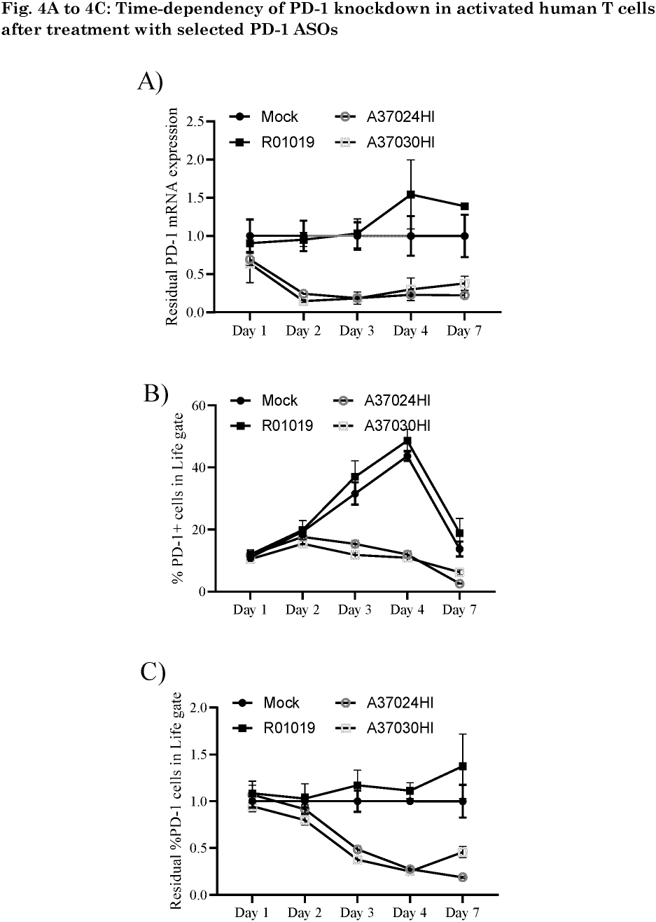

Furthermore, the time-dependency of PD-1 knockdown in activated human T cells after treatment with the PD-1-specific ASOs A37024HI (SEQ ID NO.22) and A37030HI (SEQ ID NO.27) was investigated. Therefore, T cells were isolated, activated and either not treated with an ASO (mock), treated with the control oligonucleotide R01019 or one of the PD-1-specific ASOs A37024HI (SEQ ID NO.22) and A37030HI (SEQ ID NO.27) at a final concentration of 5 µM. PD-1 mRNA and protein expression was assessed on day 1, 2, 3, 4, 5, and 7 after start of ASO treatment. As shown in FIG. 4A and Table 6, residual PD-1 mRNA expression was potently reduced from day 2 to day 7 after start of treatment by the PD-1-specific ASOs A37024HI (SEQ ID NO.22) and A37030HI (SEQ ID NO.27), whereas the control oligonucleotide R01019 had no negative impact on PD-1 mRNA expression. FIGS. 4B and 4C and Table 7 show that PD-1 protein expression (as assessed by flow cytometry) was also potently reduced in activated human T cells that had been treated with the PD-1-specific ASOs A37024HI (SEQ ID NO.22) or A37030HI (SEQ ID NO.27).

TABLE 6

Time-dependency of PD-1 mRNA knockdown in activated human T cells after treatment with selected PD-1 ASOs.
Inhibition (%) of PD-1 mRNA expression

| | R01019 | A37024HI | A37030HI |
|---|---|---|---|
| Day 1 | 9.54 | 30.75 | 36.34 |
| Day 2 | 4.80 | 75.65 | 85.41 |
| Day 3 | -3.25 | 81.32 | 81.48 |
| Day 4 | -54.20 | 76.98 | 69.89 |
| Day 7 | -38.85 | 77.82 | 62.09 |

TABLE 7

Time-dependency of reduction of PD-1+ cells in Life gate in activated human T cells after treatment with selected PD-1 ASOs.
Reduction (%) of PD-1+ cells in Lifegate

| | R01019 | A37024HI | A37030HI |
|---|---|---|---|
| Day 1 | -8.43 | -6.93 | 5.42 |
| Day 2 | -2.93 | 8.62 | 20.00 |
| Day 3 | -17.09 | 51.37 | 62.45 |
| Day 4 | -11.29 | 72.54 | 75.00 |
| Day 7 | -37.38 | 81.19 | 54.42 |

### Example 5: Persistency of PD-1 Target Knockdown in Activated Human T Cells After ASO Treatment, Stringent Washing and Re-Stimulation

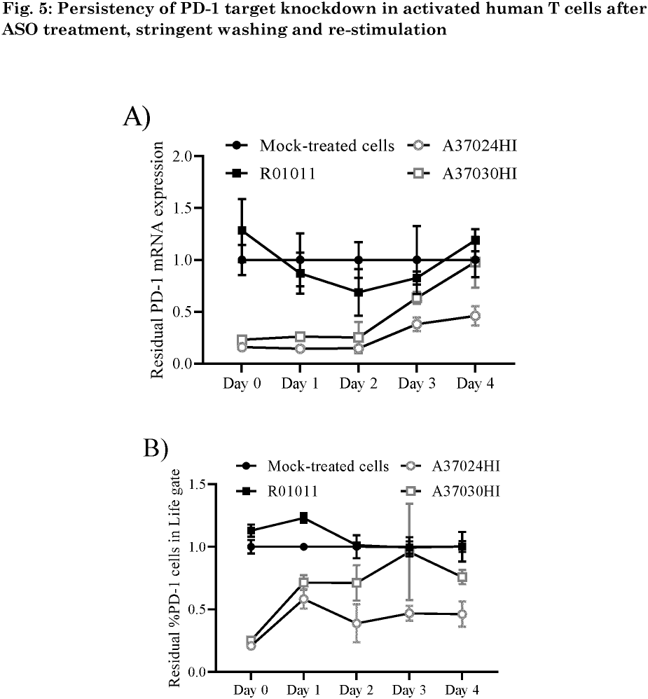

Next the persistency of PD-1 target knockdown in activated human T cells was investigated. Therefore, T cells were isolated and activated. Three days later, no ASO was added to cells (mock), the control oligonucleotide R01011 or the PD-1-specific ASOs A37024HI (SEQ ID NO.22) or A37030HI (SEQ ID NO.27) were added to a final concentration of 5 µM. Three days after addition of ASOs, cells were harvested, stringently washed and reseeded. In order to induce the expression of PD-1, cells were re-stimulated with CD3/CD28 antibodies. PD-1 mRNA and protein expression were assessed on the day of re-stimulation (day 0), and on day 1, 2, 3, and 4 after re-stimulation. As shown in FIG. 5A and Table 8, PD-1 mRNA expression was potently reduced after treatment with the PD-1-specific ASOs A37024HI (SEQ ID NO.22) and A37030HI (SEQ ID NO.27) on day 0, 1, 2, 3 and - only after treatment with A37024HI (SEQ ID NO.22) - also on day 4. Accordingly, as shown in FIG. 5B and Table 9, protein expression was potently reduced on day 0, 1, 2, 3, and day 4 when cells had been treated with A37024HI (SEQ ID NO.22) and on day 0, 1, and 2 when cells had been treated with A37030HI (SEQ ID NO.27).

TABLE 8

Persistency of PD-1 mRNA knockdown in activated human T cells after ASO treatment, stringent washing and re-stimulation.
Inhibition (%) of PD-1 mRNA expression

| After re-stimulation | R01011 | A37024HI | A37030HI |
|---|---|---|---|
| Day 0 | -28.33 | 84.12 | 76.93 |
| Day 1 | 12.91 | 85.50 | 73.85 |
| Day 2 | 31.32 | 85.26 | 74.79 |
| Day 3 | 17.48 | 61.91 | 36.64 |
| Day 4 | -18.97 | 53.92 | 2.02 |

TABLE 9

Persistency of PD-1 protein knockdown in activated human T cells after ASO treatment, stringent washing and re-stimulation.
Reduction (%) of PD-1+ cells in Lifegate

| After re-stimulation | R01011 | A37024HI | A37030HI |
|---|---|---|---|
| Day 0 | -12.98 | 79.18 | 74.96 |
| Day 1 | -23.05 | 41.66 | 28.58 |
| Day 2 | -1.30 | 61.14 | 28.86 |
| Day 3 | 0.54 | 53.18 | 4.13 |
| Day 4 | -0.25 | 53.80 | 24.20 |

### Example 6: Comparison of the Effects of a PD-1-Specific ASO and a PD-1-Specific Self-Delivering Small Interfering RNA in Activated Human T Cells

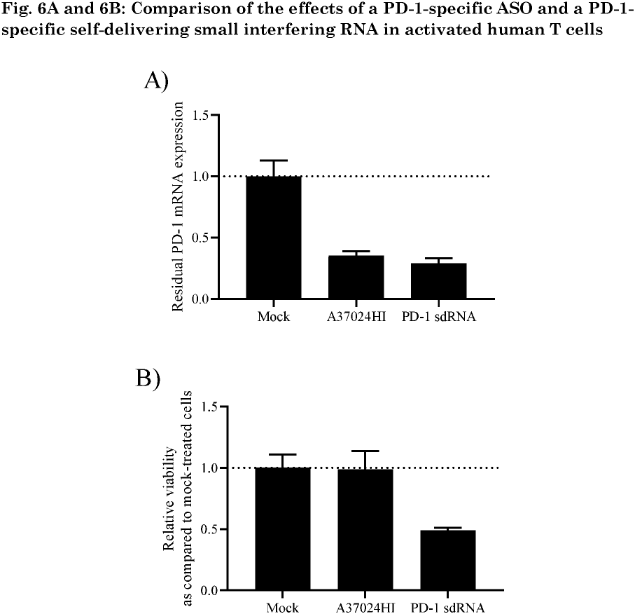

The potent PD-1-specific ASO A37024HI (SEQ ID NO.22) was compared to a commercially available PD-1-specific self-delivering small interfering RNA (sdRNA) in activated human T cells. Therefore, T cells were isolated, activated and either not treated or treated with A37024HI to a final concentration of 5 µM or a PD-1-specific sdRNA to a final concentration of 2 µM. PD-1 mRNA expression was assessed three days after start of treatment and we assessed intracellular adenosine triphosphate (ATP) content as a measure for cellular viability four days after start of treatment. As shown in FIG. 6A and Table 10, both compounds reduced PD-1 mRNA expression to a similar extend. In strong contrast, while A37024HI (SEQ ID NO.22) had no impact on cellular viability, the PD-1 sdRNA reduced viability by >50% as compared to mock-treated cells (FIG. 6B and Table 11). In conclusion, PD-1-specific ASOs potently inhibit PD-1 expression without cytotoxic effects in human activated T cells.

TABLE 10

Comparison of the inhibition (%) of PD-1 mRNA expression by a PD-1-specific ASO and a PD-1-specific sdRNA in activated human T cells.

| Compound | Inhibition (%) of PD-1 mRNA expression |
|---|---|
| A37024HI | 64.55 |
| PD-1 sdRNA | 71.05 |

TABLE 11

Comparison of the reduction (%) of cellular viability by a PD-1-specific ASO and a PD-1-specific sdRNA in activated human T cells.

| Compound | Reduction (%) of viability |
|---|---|
| A37024HI | 1.35 |
| PD-1 sdRNA | 51.04 |

1. Antisense oligonucleotide comprising 10 to 25 nucleotides, wherein at least one of the nucleotides is modified, and the antisense oligonucleotide hybridizes with a nucleic acid sequence of Programmed Cell Death 1 (PD-1) of SEQ ID NO.1, wherein the antisense oligonucleotide inhibits at least 30% of the PD1 expression in a cell compared to an untreated cell.

2. Antisense oligonucleotide according to claim 1, wherein the modified nucleotide is selected from the group consisting of a bridged nucleic acid such as LNA, cET, ENA, 2′Fluoro modified nucleotide, 20-Methyl modified nucleotide and a combination thereof.

3. Antisense oligonucleotide according to claim 1 or 2, wherein the oligonucleotide hybridizes within the region of from position 600 to position 899 of SEQ ID NO.1, within the region of from position 1500 to position 1799 of SEQ ID NO.1, within the region of from position 7800 to position 8099 of SEQ ID NO.1, within the region of from position 8700 to position 8999 of SEQ ID NO.1, within the region of from position 7500 to position 7799 of SEQ ID NO.1, within the region of from position 6000 to position 6299 of SEQ ID NO.1, within the region of from position 3000 to position 3299 of SEQ ID NO.1, within the region of from position 5100 to position 5399 of SEQ ID NO.1, within the region of from position 4500 to position 4799 of SEQ ID NO.1, within the region of from position 0 to position 299 of SEQ ID NO.1, within the region of from position 300 to position 599 of SEQ ID NO.1, within the region of from position 900 to position 1199 of SEQ ID NO.1, within the region of from position 1200 to position 1499 of SEQ ID NO.1, within the region of from position 1800 to position 2099 of SEQ ID NO.1, within the region of from position 2100 to position 2399 of SEQ ID NO.1, within the region of from position 2400 to position 2699 of SEQ ID NO.1, within the region of from position 2700 to position 2999 of SEQ ID NO.1, within the region of from position 3300 to position 3599 of SEQ ID NO.1, within the region of from position 3600 to position 3899 of SEQ ID NO.1, within the region of from position 3900 to position 4199 of SEQ ID NO.1, within the region of from position 4200 to position 4499 of SEQ ID NO.1, within the region of from position 4800 to position 5099 of SEQ ID NO.1, within the region of from position 5400 to position 5699 of SEQ ID NO.1, within the region of from position 5700 to position 5999 of SEQ ID NO.1, within the region of from position 6300 to position 6599 of SEQ ID NO.1, within the region of from position 6600 to position 6899 of SEQ ID NO.1, within the region of from position 6900 to position 7199 of SEQ ID NO.1, within the region of from position 7200 to position 7499 of SEQ ID NO.1, within the region of from position 8100 to position 8399 of SEQ ID NO.1, within the region of from position 8400 to position 8699 of SEQ ID NO.1 or within the region of from position 9000 to position 9299 of SEQ ID NO.1 or a combination thereof.

4. Antisense oligonucleotide according to any one of claims 1 to 3, wherein the modified nucleotide(s) is/are located at the 5′- or 3′-end, at the 5′- and 3′-end of the oligonucleotide, within the antisense oligonucleotide or a combination thereof.

5. Antisense oligonucleotide according to any one of claims 1 to 4, wherein the oligonucleotide comprises a sequence selected from the group consisting of SEQ ID NO. 22, SEQ ID NO.27, SEQ ID NO.29, SEQ ID NO.18, SEQ ID NO.20, SEQ ID NO.16, SEQ ID NO.14, SEQ ID NO.34, SEQ ID NO.42, SEQ ID NO.20, SEQ ID NO.23, SEQ ID NO.40 and a combination thereof.

6. Antisense oligonucleotide according to any one of claims 1 to 5, wherein the oligonucleotide is selected from the group consisting of
+C*+G*+T*C*G*T*A*A*A*G*C*C*A*A*+G*+G*+T (SEQ ID NO.22; A37024HI);
+T*+G*+A*G*A*G*T*C*T*T*G*T*C*C*+G*+G*+C (SEQ ID NO.27; A37030HI);
+C*+G*+A*A*T*G*G*C*G*A*A*C*G*C*+A*+G*+T (SEQ ID NO.29; A37032HI);
+T*+G*+G*A*C*G*G*C*C*T*G*C*A*A*+T*+G*+G (SEQ ID NO.18; A37019HI);
+G*G*+A*A*C*G*C*C*T*G*T*A*C*C*+T*+T (SEQ ID NO.20; A37021HI);
+C*+A*+T*A*C*T*C*C*G*T*C*T*G*C*+T*+C*+A (SEQ ID NO.16; A37017HI);
+C*+T*+T*T*G*A*T*C*T*G*C*G*C*C*+T*+T*+G (SEQ ID NO.14; A37015HI);
+C*G*+G*C*A*T*C*T*C*T*G*A*C*C*G*+T*+G (SEQ ID NO.34; A37037HI);
+C*+G*+A*G*A*T*G*C*C*A*T*G*C*A*+A*+C*+G (SEQ ID NO.42; A37046HI);
+G*G*+A*A*C*G*C*C*T*G*T*A*C*C*+T*+T (SEQ ID NO.20; A37022HI);
+G*+A*+A*C*T*G*T*C*C*T*C*A*C*T*+C*+G*+A (SEQ ID NO.23; A37025HI);
+G*+C*+T*G*A*C*A*A*G*C*G*C*T*C*G*+C*+C (SEQ ID NO.40; A37043HI)
and a combination thereof, wherein + indicates a LNA-modified nucleotide and * indicates phosphorothioate.

7. Pharmaceutical composition comprising the oligonucleotide according to any one of claims 1 to 6 and a pharmaceutically acceptable excipient.

8. Antisense oligonucleotide according to any one of claims 1 to 6 or the pharmaceutical composition according to claim 7 for use in T cell therapy.

9. Antisense oligonucleotide according to any one of claims 1 to 6 or the pharmaceutical composition according to claim 7 for use in a method of preventing and/or treating a malignant tumor, a benign tumor and/or an infectious disease.

10. Antisense oligonucleotide or pharmaceutical composition for use according to claim 8 or 9, wherein the tumor is selected from the group consisting of solid tumors, blood born tumors, leukemias, tumor metastasis, hemangiomas, acoustic neuromas, neurofibromas, trachomas, pyogenic granulomas, psoriasis, astrocytoma, blastoma, Ewing’s tumor, craniopharyngioma, ependymoma, medulloblastoma, glioma, hemangioblastoma, Hodgkin’s lymphoma, mesothelioma, neuroblastoma, non-Hodgkin’s lymphoma, pinealoma, retinoblastoma, sarcoma, seminoma, and Wilms’ tumor, bile duct carcinoma, bladder carcinoma, brain tumor, breast cancer, bronchogenic carcinoma, carcinoma of the kidney, cervical cancer, choriocarcinoma, choroid carcinoma, cystadenocarcinoma, embryonal carcinoma, epithelial carcinoma, esophageal cancer, cervical carcinoma, colon carcinoma, colorectal carcinoma, endometrial cancer, gallbladder cancer, gastric cancer, head cancer, liver carcinoma, lung carcinoma, medullary carcinoma, neck cancer, non-small-cell bronchogenic/lung carcinoma, ovarian cancer, pancreas carcinoma, papillary carcinoma, papillary adenocarcinoma, prostate cancer, small intestine carcinoma, prostate carcinoma, rectal cancer, renal cell carcinoma, skin cancer, small-cell bronchogenic/lung carcinoma, squamous cell carcinoma, sebaceous gland carcinoma, testicular carcinoma, uterine cancer or a combination thereof, or wherein the infectious disease is selected from the group consisting of a Hepatitis B infection, a Hepatitis A infection, a Cytomegalovirus infection, an Epstein-Barr-Virus infection, an Adenovirus infection or a combination thereof.

11. Use of the antisense oligonucleotide according to any one of claims 1 to 6 or the pharmaceutical composition according to claim 7 for reducing expression of PD-1 in an isolated immune cell in preparation for cell therapy.

12. Method for reducing expression of PD-1 RNA in an isolated immune cell in preparation for cell therapy, comprising:
incubating the isolated immune cell comprising the PD-1 RNA with an antisense oligonucleotide according to any one of claims 1 to 6 or the pharmaceutical composition according to claim 7 without use of a transfection means, wherein the antisense oligonucleotide is administered to the isolated immune cell at least once in a time period of day 0 to day 21, the antisense oligonucleotide hybridizes with the PD-1 RNA and reduces the expression of PD-1, reduces the function and/or activity of the PD-1, or a combination thereof up to 2 weeks from day 0 of the incubation with the antisense oligonucleotide.

13. Method according to claim 12, wherein the isolated immune cell is genetically modified by a gene transfer technology before or after incubating the immune cell with the antisense oligonucleotide, for example wherein the immune cell is permanently or transiently modified.

14. Method according to claim 12 or 13, wherein the isolated, genetically modified immune cell is expanded before or after incubating the immune cell with the antisense oligonucleotide.

15. Method according to any one of claims 12 to 14, wherein the immune cell is selected from the group consisting of a T cell, a dendritic cell, a natural killer (NK) cell, a peripheral blood mononuclear cell (PBMC), a hematopoietic stem cell, a B cell and a combination thereof.


SEQUENCE LISTING (408 sequences)

SEQ ID NO: 1 - DNA, 9026 bp, Homo sapiens
agtttcccttccgctcacctccgcctgagcagtggagaaggcggcactctggtggggctgctccaggcatgcagatcccacaggcgccctggccagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcaggctggccagaaccccaaaccttggccgtggggagtctgcgtggcggctctgccttgcccaggcatccttggtcctcactcgagttttcctaaggatgggatgagccccatgtgggactaaccttggctttacgacgtcaaagtttagatgagctggtgatatttttctcattatatccaaagtgtacctgttcgagtgaggacagttcttctgtctccaggatccctcctgggtggggattgtgcccgcctgggtctctgcccagattccagggctctccccgagccctgttcagaccatccgtgggggaggccttggcctcactctcccggatcgaggagagagggagcctcttcctgggctgcccgtgaccctgggccctctgtgtacactgtgaccacagcccgctcctggaccctctgtgcccggctggccctctgtgcccagccagcctgcacctggggatgccaaggcctggggagggtggtttcacccaggccaagcctaagacagtccctctgggccctgctgggtaccggggtgtgacaccactgggaggacaagatgaggggcacccctggggccgccctgacaccccctcgaggctcctgccccgggggtcctggtgccccttcactgtggcaggcgactgggggttccccacctcggcccctctcccggggcctgctccccggcacctgaggcagcatccttgtcagggccgtgccttcctgcctcagcgccacctcttaaggttggcccgtgggtcactcaggactcagaactggagattctgggcaaaaggcaaagagcaaagggccaaaaggcatcccagggagacgactgcgggggaaccagagggcagaggggcgctcgtcacaggggagggggagctgagcgaggcaggaggggagccgagcctctccccccgtgtcccggctcttcaggcacgccctcgggacgccaccctccccgacccaggcgggaaagataagagcaaggtgtccgcagcctgacactcgtgcctcaggtgcccgcgcttgtgccggacaagactctcacaggtggcatgcctcggtttccccactggtaacagcacagggcactcagcaaggcgcagtgggcatgactggggtcctgtgggtcctgacccagatgtggccaccccggccgcagtggtcttcattccaggatgcctcttttccctcctgatctattcactgcgttcgccattcggtcattcccggggccaccactcccacctcaggtgtgtgcttcccttgtgttttatgagatatccccaacccggctgcttattggccccgtccgagggcaggagcataaataagagcctctgctttggcgtgggaccactgtgagctccagtcagcgctgccactgctgagctctgggccttcgacaggacttggccccttactgacttctccgtgtgctttgggtcatgggtgaggacgcctcctggcaaggctgcgtcctgaggattaaatcgggtcatctgtgaaaactacccagcccagcccctgacacttttttgtttgtttcttttagtgacagggtcttgctctgtcacccaggctggagtgcagtggtgtgatctcggctcactcgacctccggggctcaagcaattctcccacctctgcctccagagtagctgggactataggcacgtgccaccctgccaggctaatttcttccattttttttagagacagggtctcgctatgttccccaggctgggctcaaatggtcctcccacctcagcctccccaagtactgggattacaggcataagccactgcatctggcctccatgacacatatttttaaagtctgatttttaaagtcaaacttttgaagtcagattttaaacggactattttgaaaaatatacaaaaacgtttaaaaacaatgaatatccctcacctagaatcaataactaagaatattgacacatttgctttggggactgggcggctggagctgccatgacaaagctccgccgaccgagtggcttttaaacagagcctgccctctcgccgactgagggctggacgtgcaggatggagctccgcagggtcggctcccctgtgctctgaggggctctgctcagcctctcccggctgtggcttaaaaacagagcctgtcctcccgccgtggggggctggacatgcaggaccgaggggccacagggtcggctccctgtgctccgagagggctctgctcagcttctcctggctggggggttttgtggccaccctctgtgttcctgggttcagaagcatcccccaggctctgccttcatctctgcacgggtgactctgtacaggaagccaggcctgctggtcaatggccacccagccctgtgccctcatcttacctagtcccagctgccgtcaccctattcctaataaggccgccttctgaggtcatggggttaggacttccacataggaatctgtggggacacggttcggcccacagcccttcccacctccacacacacacacgactgtgaggagttggaagacctcactcctcacccctgccaggtcctctagggacaagctcgctgtcctcatcccagcacagcccgtgggacggtttccttgtccctaatgggaccacggtcagagatgccgggtctggtctgggccagcaggttcctccgcccggggcaggcagccttcttctgtgcgcttctggaaagcaatgtcctgtaatgcggtctctctgcgggagcacccccaccgccacctcacaggcctgttccacagccccgggatgggctctgtctccctcctgaccctgcatagggcacagccctctctcatcaacccacgatcctacgtggatccgagagggagcacctggggaaacaatggaatcccatagaaacaccccaaatctaacttgatccaggaccagccagtggtcacttctgaatattcaccttcctagtagacactaccagccaagggaggccaggaagccttcctggaggaggtggcctgaggactggggtgaggcaggccctgcgtgggggtcgccacccagcacccccacactgggtgggagccagtctctgagactggctgggggaggtgggagagggggctgcttgaactgcagacaccgaggtctagcccccaccccacccagccagttggtggaggcaggggaggccgaggggcccagctggacctgctccccggggtggattccaaaataggggggttggggggggcggaacaggagcccagggtcctggcttgaggcccagtggctgagggctggtgcaagccagacaggaaaagggttgagcctgtcagcgccagcacagatcaagtcaggagcaggtccctccaccaatgtgtgcaaataaatagcagctaagtttccagttacaagaacaatgcacagatggtcccagggacattgcggtgtggacacacagcggccattgtcctgtcgccagcacctcgccctacagctggggggtcccttagcacttcctagccatgcagggtccctgctcacagtacccgtgatgacttctgttcctcacctgcctgtctgtcccgacagctgcatggcagccctggcctgggagatggagaccccgaggggctgcctgcggtggtggggcccctgggtccccactgcattcccagaaacccagagggcagggcatttcccctgctctgtgccgagtccacccagccccagcctaggcccagtaagggctgcagcccaccctgtcccaggctgcctcccaggagccctcttggccctgatgccagaagcccatcttcctccattcaggcaggtctctgagtgccctggcctggctgcctgctggccctgagagtcacactaccccacagccctccttggtcaaaatccactctggagtggctggaagattccccgggcccacgccgcacacgcctatgcagggagcttcccctggccggccggcagacaagggcggtctcagagagggggctcacctcagcagccccttgtgtagctggccctcgcccctgccacctctgggaacaccaccaggaagctgggggacaggcacgcaggtgaaggaggcgagcgcttgtcagccgggaggccatgggcacagagggaacagggacaccctgggtggcctcaaggtcacttcaaacccctcactcgtcccctgggagggtgcccagtgaggttggcactaggagttggtcctggtcacatgacagacccacccacctctggtgtccagccagcacgccgtgggccagcctggctgcagggacacgagggcagcagccccctcctcctctgagctggttgctccttgagtcatcaccaccgcctgccacggaggccgcctgtcccaggaagcagagggaccgcagctgtggcaaccagggcctggtctctgtgtcacctcgctggggggccgtgcccaggcctgagacggaactgagtgacagtgcactgggtctgacagtgtggggctggcgccatgtttggggaaccctgtggcatgggacctgtgggtgagccgggaaaatcaccccgttgcatggcatctcgggcctggatcttaagcgcctgtgttggtgcctccgcctggcggaagagccgcgacccccacgttgccatgcgggtatcccaagccctgaccctggcaggcatatgtttcaggaggtccttgtcttgggagcccagggtcgggggccccgtgtctgtccacatccgagtcaatggcccatctcgtctctgaagcatctttgctgtgagctctagtccccactgtcttgctggaaaatgtggaggccccactgcccactgcccagggcagcaatgcccataccacgtggtcccagctccgagcttgtcctgaaaagggggcaaagactggaccctgagcctgccaaggggccacactcctcccagggctggggtctccatgggcagccccccacccacccagaccagttacactcccctgtgccagagcagtgcagacaggaccaggccaggatgcccaagggtcaggggctggggatgggtagcccccaaacagccctttctgggggaactggcctcaacggggaagggggtgaaggctcttagtaggaaatcagggagacccaagtcagagccaggtgctgtgcagaagctgcagcctcacgtagaaggaagaggctctgcagtggaggccagtgcccatccccgggtggcagaggccccagcagagacttctcaatgacattccagctggggtggcccttccagagcccttgctgcccgagggatgtgagcaggtggccggggaggctttgtggggccacccagccccttcctcacctctctccatctctcagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttgtgcccttccagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgaggtaacgtcatcccagcccctcggcctgccctgccctaaccctgctggcggccctcactcccgcctccccttcctccacccttccctcaccccaccccacctccccccatctccccgccaggctaagtccctgatgaaggcccctggactaagaccccccacctaggagcacggctcagggtcggcctggtgaccccaagtgtgtttctctgcagggacaataggagccaggcgcaccggccagcccctggtgagtctcactcttttcctgcatgatccactgtgccttccttcctgggtgggcagaggtggaaggacaggctgggaccacacggcctgcaggactcacattctattatagccaggaccccacctccccagcccccaggcagcaacctcaatccctaaagccatgatctggggccccagcccacctgcggtctccgggggtgcccggcccatgtgtgtgcctgcctgcggtctccaggggtgcctggcccacgcgtgtgcccgcctgcggtctctgggggtgcccggcccacatatgtgcctgcctgcggtctccaggtgtgcccggcccatgcgtgtgcccacctgcgagggcgtggggtgggcttggtcatttcttatcttacattggagacaggagagcttgaaaagtcacattttggaatcctaaatctgcaagaatgccagggacatttcagagggggacattgagccagagaggaggggtggtgtccccagatcacacagagggcagtggtgggacagctcagggtaagcagctcatagtggggggcccaggttcggtgccggtactgcagccaggctgtggagccgcgggcctccttcctgcggtgggccgtggggctgactccctctccctttctcctcaaagaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaagga

SEQ ID NO: 2 - DNA, 17 bp, Artificial Sequence
  antisense oligonucleotide
caccagagtgccgcctt

SEQ ID NO: 3 - DNA, 17 bp, Artificial Sequence
  antisense oligonucleotide
cggtcaccacgagcagg

SEQ ID NO: 4 - DNA, 17 bp, Artificial Sequence
  antisense oligonucleotide
tcggtcaccacgagcag

SEQ ID NO: 5 - DNA, 16 bp, Artificial Sequence
  antisense oligonucleotide
ccttcggtcaccacga

SEQ ID NO: 6 - DNA, 17 bp, Artificial Sequence
  antisense oligonucleotide
cgaagctctccgatgtg

SEQ ID NO: 7 - DNA, 17 bp, Artificial Sequence
  antisense oligonucleotide
gcacgaagctctccgat

SEQ ID NO: 8 - DNA, 17 bp, Artificial Sequence
  antisense oligonucleotide
ccagtttagcacgaagc

SEQ ID NO: 9 - DNA, 17 bp, Artificial Sequence
  antisense oligonucleotide
gtaccagtttagcacga

SEQ ID NO: 10 - DNA, 17 bp, Artificial Sequence
  antisense oligonucleotide
atgcggtaccagtttag

SEQ ID NO: 11 - DNA, 17 bp, Artificial Sequence
  antisense oligonucleotide
cttgtccgtctggttgc

SEQ ID NO: 12 - DNA, 17 bp, Artificial Sequence
  antisense oligonucleotide
tgacacggaagcggcag

SEQ ID NO: 13 - DNA, 17 bp, Artificial Sequence
  antisense oligonucleotide
acagaggtaggtgccgc

SEQ ID NO: 14 - DNA, 17 bp, Artificial Sequence
  antisense oligonucleotide
ctttgatctgcgccttg

SEQ ID NO: 15 - DNA, 17 bp, Artificial Sequence
  antisense oligonucleotide
acgacaccaaccaccag

SEQ ID NO: 16 - DNA, 17 bp, Artificial Sequence
  antisense oligonucleotide
catactccgtctgctca

SEQ ID NO: 17 - DNA, 17 bp, Artificial Sequence
  antisense oligonucleotide
ccattccgctaggaaag

SEQ ID NO: 18 - DNA, 17 bp, Artificial Sequence
  antisense oligonucleotide
tggacggcctgcaatgg

SEQ ID NO: 19 - DNA, 17 bp, Artificial Sequence
  antisense oligonucleotide
ccacggcgccttcagcc

SEQ ID NO: 20 - DNA, 16 bp, Artificial Sequence
  antisense oligonucleotide
ggaacgcctgtacctt

SEQ ID NO: 21 - DNA, 17 bp, Artificial Sequence
  antisense oligonucleotide
tcgagtgaggaccaagg

SEQ ID NO: 22 - DNA, 17 bp, Artificial Sequence
  antisense oligonucleotide
cgtcgtaaagccaaggt

SEQ ID NO: 23 - DNA, 17 bp, Artificial Sequence
  antisense oligonucleotide
gaactgtcctcactcga

SEQ ID NO: 24 - DNA, 17 bp, Artificial Sequence
  antisense oligonucleotide
ctctctcctcgatccgg

SEQ ID NO: 25 - DNA, 17 bp, Artificial Sequence
  antisense oligonucleotide
cgtgcctgaagagccgg

SEQ ID NO: 26 - DNA, 17 bp, Artificial Sequence
  antisense oligonucleotide
tgtccggcacaagcgcg

SEQ ID NO: 27 - DNA, 17 bp, Artificial Sequence
  antisense oligonucleotide
tgagagtcttgtccggc

SEQ ID NO: 28 - DNA, 17 bp, Artificial Sequence
  antisense oligonucleotide
aacgcagtgaatagatc

SEQ ID NO: 29 - DNA, 17 bp, Artificial Sequence
  antisense oligonucleotide
cgaatggcgaacgcagt

SEQ ID NO: 30 - DNA, 17 bp, Artificial Sequence
  antisense oligonucleotide
aagtcctgtcgaaggcc

SEQ ID NO: 31 - DNA, 17 bp, Artificial Sequence
  antisense oligonucleotide
agccactcggtcggcgg

SEQ ID NO: 32 - DNA, 17 bp, Artificial Sequence
  antisense oligonucleotide
aagccactcggtcggcg

SEQ ID NO: 33 - DNA, 17 bp, Artificial Sequence
  antisense oligonucleotide
gcggccttattaggaat

SEQ ID NO: 34 - DNA, 17 bp, Artificial Sequence
  antisense oligonucleotide
cggcatctctgaccgtg

SEQ ID NO: 35 - DNA, 17 bp, Artificial Sequence
  antisense oligonucleotide
cgcagagagaccgcatt

SEQ ID NO: 36 - DNA, 17 bp, Artificial Sequence
  antisense oligonucleotide
ctcggatccacgtagga

SEQ ID NO: 37 - DNA, 17 bp, Artificial Sequence
  antisense oligonucleotide
tgatctgtgctggcgct

SEQ ID NO: 38 - DNA, 17 bp, Artificial Sequence
  antisense oligonucleotide
cgacaggacaatggccg

SEQ ID NO: 39 - DNA, 17 bp, Artificial Sequence
  antisense oligonucleotide
ataggcgtgtgcggcgt

SEQ ID NO: 40 - DNA, 17 bp, Artificial Sequence
  antisense oligonucleotide
gctgacaagcgctcgcc

SEQ ID NO: 41 - DNA, 17 bp, Artificial Sequence
  antisense oligonucleotide
ggaccaactcctagtgc

SEQ ID NO: 42 - DNA, 17 bp, Artificial Sequence
  antisense oligonucleotide
cgagatgccatgcaacg

SEQ ID NO: 43 - DNA, 17 bp, Artificial Sequence
  antisense oligonucleotide
cttctacgtgaggctgc

SEQ ID NO: 44 - DNA, 17 bp, Artificial Sequence
  antisense oligonucleotide
tgagccgtgctcctagg

SEQ ID NO: 45 - DNA, 17 bp, Artificial Sequence
  antisense oligonucleotide
taccggcaccgaacctg

SEQ ID NO: 46 - DNA, 16 bp, Artificial Sequence
  antisense oligonucleotide
taccggcaccgaacct

SEQ ID NO: 47 - DNA, 16 bp, Artificial Sequence
  antisense oligonucleotide
gcagtaccggcaccga

SEQ ID NO: 48 - DNA, 18 bp, Artificial Sequence
  antisense oligonucleotide
tcggtcaccacgagcagg

SEQ ID NO: 49 - DNA, 18 bp, Artificial Sequence
  antisense oligonucleotide
ccttcggtcaccacgagc

SEQ ID NO: 50 - DNA, 18 bp, Artificial Sequence
  antisense oligonucleotide
aggtgaaggtggcgttgt

SEQ ID NO: 51 - DNA, 18 bp, Artificial Sequence
  antisense oligonucleotide
caggtgaaggtggcgttg

SEQ ID NO: 52 - DNA, 18 bp, Artificial Sequence
  antisense oligonucleotide
aagctctccgatgtgttg

SEQ ID NO: 53 - DNA, 18 bp, Artificial Sequence
  antisense oligonucleotide
cgaagctctccgatgtgt

SEQ ID NO: 54 - DNA, 18 bp, Artificial Sequence
  antisense oligonucleotide
agcacgaagctctccgat

SEQ ID NO: 55 - DNA, 18 bp, Artificial Sequence
  antisense oligonucleotide
accagtttagcacgaagc

SEQ ID NO: 56 - DNA, 18 bp, Artificial Sequence
  antisense oligonucleotide
taccagtttagcacgaag

SEQ ID NO: 57 - DNA, 18 bp, Artificial Sequence
  antisense oligonucleotide
gtaccagtttagcacgaa

SEQ ID NO: 58 - DNA, 18 bp, Artificial Sequence
  antisense oligonucleotide
catgcggtaccagtttag

SEQ ID NO: 59 - DNA, 18 bp, Artificial Sequence
  antisense oligonucleotide
tcatgcggtaccagttta

SEQ ID NO: 60 - DNA, 18 bp, Artificial Sequence
  antisense oligonucleotide
ttgtccgtctggttgctg

SEQ ID NO: 61 - DNA, 18 bp, Artificial Sequence
  antisense oligonucleotide
cttgtccgtctggttgct

SEQ ID NO: 62 - DNA, 18 bp, Artificial Sequence
  antisense oligonucleotide
tgtgacacggaagcggca

SEQ ID NO: 63 - DNA, 18 bp, Artificial Sequence
  antisense oligonucleotide
cagttgtgtgacacggaa

SEQ ID NO: 64 - DNA, 18 bp, Artificial Sequence
  antisense oligonucleotide
gcagttgtgtgacacgga

SEQ ID NO: 65 - DNA, 18 bp, Artificial Sequence
  antisense oligonucleotide
tcatgtggaagtcacgcc

SEQ ID NO: 66 - DNA, 18 bp, Artificial Sequence
  antisense oligonucleotide
gctcatgtggaagtcacg

SEQ ID NO: 67 - DNA, 18 bp, Artificial Sequence
  antisense oligonucleotide
cacagaggtaggtgccgc

SEQ ID NO: 68 - DNA, 18 bp, Artificial Sequence
  antisense oligonucleotide
ctttgatctgcgccttgg

SEQ ID NO: 69 - DNA, 18 bp, Artificial Sequence
  antisense oligonucleotide
tctttgatctgcgccttg

SEQ ID NO: 70 - DNA, 18 bp, Artificial Sequence
  antisense oligonucleotide
ctctctttgatctgcgcc

SEQ ID NO: 71 - DNA, 18 bp, Artificial Sequence
  antisense oligonucleotide
cgcaggctctctttgatc

SEQ ID NO: 72 - DNA, 18 bp, Artificial Sequence
  antisense oligonucleotide
ccgcaggctctctttgat

SEQ ID NO: 73 - DNA, 18 bp, Artificial Sequence
  antisense oligonucleotide
acgacaccaaccaccagg

SEQ ID NO: 74 - DNA, 18 bp, Artificial Sequence
  antisense oligonucleotide
cacgacaccaaccaccag

SEQ ID NO: 75 - DNA, 18 bp, Artificial Sequence
  antisense oligonucleotide
gagaacacaggcacggct

SEQ ID NO: 76 - DNA, 18 bp, Artificial Sequence
  antisense oligonucleotide
atagtccacagagaacac

SEQ ID NO: 77 - DNA, 18 bp, Artificial Sequence
  antisense oligonucleotide
catagtccacagagaaca

SEQ ID NO: 78 - DNA, 18 bp, Artificial Sequence
  antisense oligonucleotide
ccatagtccacagagaac

SEQ ID NO: 79 - DNA, 18 bp, Artificial Sequence
  antisense oligonucleotide
ggtcttctctcgccactg

SEQ ID NO: 80 - DNA, 18 bp, Artificial Sequence
  antisense oligonucleotide
atactccgtctgctcagg

SEQ ID NO: 81 - DNA, 18 bp, Artificial Sequence
  antisense oligonucleotide
ccattccgctaggaaaga

SEQ ID NO: 82 - DNA, 18 bp, Artificial Sequence
  antisense oligonucleotide
ccaaggaagccggtcaga

SEQ ID NO: 83 - DNA, 18 bp, Artificial Sequence
  antisense oligonucleotide
cattgagacatgagtcct

SEQ ID NO: 84 - DNA, 18 bp, Artificial Sequence
  antisense oligonucleotide
gcattgagacatgagtcc

SEQ ID NO: 85 - DNA, 18 bp, Artificial Sequence
  antisense oligonucleotide
ccttagcatgctctcata

SEQ ID NO: 86 - DNA, 19 bp, Artificial Sequence
  antisense oligonucleotide
caggcggaggtgagcggaa

SEQ ID NO: 87 - DNA, 19 bp, Artificial Sequence
  antisense oligonucleotide
gtgccgccttctccactgc

SEQ ID NO: 88 - DNA, 19 bp, Artificial Sequence
  antisense oligonucleotide
gatctgcatgcctggagca

SEQ ID NO: 89 - DNA, 19 bp, Artificial Sequence
  antisense oligonucleotide
taagaaccatcctggccgc

SEQ ID NO: 90 - DNA, 19 bp, Artificial Sequence
  antisense oligonucleotide
cttcggtcaccacgagcag

SEQ ID NO: 91 - DNA, 19 bp, Artificial Sequence
  antisense oligonucleotide
ccttcggtcaccacgagca

SEQ ID NO: 92 - DNA, 19 bp, Artificial Sequence
  antisense oligonucleotide
caggtgaaggtggcgttgt

SEQ ID NO: 93 - DNA, 19 bp, Artificial Sequence
  antisense oligonucleotide
gcaggtgaaggtggcgttg

SEQ ID NO: 94 - DNA, 19 bp, Artificial Sequence
  antisense oligonucleotide
gagaagctgcaggtgaagg

SEQ ID NO: 95 - DNA, 19 bp, Artificial Sequence
  antisense oligonucleotide
gttggagaagctgcaggtg

SEQ ID NO: 96 - DNA, 19 bp, Artificial Sequence
  antisense oligonucleotide
gtgttggagaagctgcagg

SEQ ID NO: 97 - DNA, 19 bp, Artificial Sequence
  antisense oligonucleotide
atgtgttggagaagctgca

SEQ ID NO: 98 - DNA, 19 bp, Artificial Sequence
  antisense oligonucleotide
gatgtgttggagaagctgc

SEQ ID NO: 99 - DNA, 19 bp, Artificial Sequence
  antisense oligonucleotide
gaagctctccgatgtgttg

SEQ ID NO: 100 - DNA, 19 bp, Artificial Sequence
  antisense oligonucleotide
cgaagctctccgatgtgtt

SEQ ID NO: 101 - DNA, 19 bp, Artificial Sequence
  antisense oligonucleotide
agcacgaagctctccgatg

SEQ ID NO: 102 - DNA, 19 bp, Artificial Sequence
  antisense oligonucleotide
tagcacgaagctctccgat

SEQ ID NO: 103 - DNA, 19 bp, Artificial Sequence
  antisense oligonucleotide
tttagcacgaagctctccg

SEQ ID NO: 104 - DNA, 19 bp, Artificial Sequence
  antisense oligonucleotide
taccagtttagcacgaagc

SEQ ID NO: 105 - DNA, 19 bp, Artificial Sequence
  antisense oligonucleotide
gtaccagtttagcacgaag

SEQ ID NO: 106 - DNA, 19 bp, Artificial Sequence
  antisense oligonucleotide
catgcggtaccagtttagc

SEQ ID NO: 107 - DNA, 19 bp, Artificial Sequence
  antisense oligonucleotide
tcatgcggtaccagtttag

SEQ ID NO: 108 - DNA, 19 bp, Artificial Sequence
  antisense oligonucleotide
ctcatgcggtaccagttta

SEQ ID NO: 109 - DNA, 19 bp, Artificial Sequence
  antisense oligonucleotide
ttgtccgtctggttgctgg

SEQ ID NO: 110 - DNA, 19 bp, Artificial Sequence
  antisense oligonucleotide
cttgtccgtctggttgctg

SEQ ID NO: 111 - DNA, 19 bp, Artificial Sequence
  antisense oligonucleotide
gcttgtccgtctggttgct

SEQ ID NO: 112 - DNA, 19 bp, Artificial Sequence
  antisense oligonucleotide
tgtgacacggaagcggcag

SEQ ID NO: 113 - DNA, 19 bp, Artificial Sequence
  antisense oligonucleotide
gttgtgtgacacggaagcg

SEQ ID NO: 114 - DNA, 19 bp, Artificial Sequence
  antisense oligonucleotide
gcagttgtgtgacacggaa

SEQ ID NO: 115 - DNA, 19 bp, Artificial Sequence
  antisense oligonucleotide
ggcagttgtgtgacacgga

SEQ ID NO: 116 - DNA, 19 bp, Artificial Sequence
  antisense oligonucleotide
ctcatgtggaagtcacgcc

SEQ ID NO: 117 - DNA, 19 bp, Artificial Sequence
  antisense oligonucleotide
tgaccacgctcatgtggaa

SEQ ID NO: 118 - DNA, 19 bp, Artificial Sequence
  antisense oligonucleotide
ccacagaggtaggtgccgc

SEQ ID NO: 119 - DNA, 19 bp, Artificial Sequence
  antisense oligonucleotide
tctttgatctgcgccttgg

SEQ ID NO: 120 - DNA, 19 bp, Artificial Sequence
  antisense oligonucleotide
ctctttgatctgcgccttg

SEQ ID NO: 121 - DNA, 19 bp, Artificial Sequence
  antisense oligonucleotide
cgcaggctctctttgatct

SEQ ID NO: 122 - DNA, 19 bp, Artificial Sequence
  antisense oligonucleotide
cacgacaccaaccaccagg

SEQ ID NO: 123 - DNA, 19 bp, Artificial Sequence
  antisense oligonucleotide
ccacgacaccaaccaccag

SEQ ID NO: 124 - DNA, 19 bp, Artificial Sequence
  antisense oligonucleotide
cagactagcagcaccaggc

SEQ ID NO: 125 - DNA, 19 bp, Artificial Sequence
  antisense oligonucleotide
agagaacacaggcacggct

SEQ ID NO: 126 - DNA, 19 bp, Artificial Sequence
  antisense oligonucleotide
cagagaacacaggcacggc

SEQ ID NO: 127 - DNA, 19 bp, Artificial Sequence
  antisense oligonucleotide
gtccacagagaacacaggc

SEQ ID NO: 128 - DNA, 19 bp, Artificial Sequence
  antisense oligonucleotide
atagtccacagagaacaca

SEQ ID NO: 129 - DNA, 19 bp, Artificial Sequence
  antisense oligonucleotide
catagtccacagagaacac

SEQ ID NO: 130 - DNA, 19 bp, Artificial Sequence
  antisense oligonucleotide
ccatagtccacagagaaca

SEQ ID NO: 131 - DNA, 19 bp, Artificial Sequence
  antisense oligonucleotide
ttctctcgccactggaaat

SEQ ID NO: 132 - DNA, 19 bp, Artificial Sequence
  antisense oligonucleotide
cttctctcgccactggaaa

SEQ ID NO: 133 - DNA, 19 bp, Artificial Sequence
  antisense oligonucleotide
catactccgtctgctcagg

SEQ ID NO: 134 - DNA, 19 bp, Artificial Sequence
  antisense oligonucleotide
acaatggtggcatactccg

SEQ ID NO: 135 - DNA, 19 bp, Artificial Sequence
  antisense oligonucleotide
aagacaatggtggcatact

SEQ ID NO: 136 - DNA, 19 bp, Artificial Sequence
  antisense oligonucleotide
aaagacaatggtggcatac

SEQ ID NO: 137 - DNA, 19 bp, Artificial Sequence
  antisense oligonucleotide
gaaagacaatggtggcata

SEQ ID NO: 138 - DNA, 19 bp, Artificial Sequence
  antisense oligonucleotide
attccgctaggaaagacaa

SEQ ID NO: 139 - DNA, 19 bp, Artificial Sequence
  antisense oligonucleotide
caaggaagccggtcagagg

SEQ ID NO: 140 - DNA, 19 bp, Artificial Sequence
  antisense oligonucleotide
ccaaggaagccggtcagag

SEQ ID NO: 141 - DNA, 19 bp, Artificial Sequence
  antisense oligonucleotide
gcagaacactggtggccaa

SEQ ID NO: 142 - DNA, 19 bp, Artificial Sequence
  antisense oligonucleotide
ctgaggaaatgcgctgacc

SEQ ID NO: 143 - DNA, 19 bp, Artificial Sequence
  antisense oligonucleotide
tctcctgaggaaatgcgct

SEQ ID NO: 144 - DNA, 19 bp, Artificial Sequence
  antisense oligonucleotide
agacatgagtcctgtggtg

SEQ ID NO: 145 - DNA, 19 bp, Artificial Sequence
  antisense oligonucleotide
tgagacatgagtcctgtgg

SEQ ID NO: 146 - DNA, 19 bp, Artificial Sequence
  antisense oligonucleotide
gcattgagacatgagtcct

SEQ ID NO: 147 - DNA, 19 bp, Artificial Sequence
  antisense oligonucleotide
tggcaggacctgaagcagt

SEQ ID NO: 148 - DNA, 19 bp, Artificial Sequence
  antisense oligonucleotide
tggacgcaggcagctctgt

SEQ ID NO: 149 - DNA, 19 bp, Artificial Sequence
  antisense oligonucleotide
gcagcagcagagattcagg

SEQ ID NO: 150 - DNA, 19 bp, Artificial Sequence
  antisense oligonucleotide
caaggccatctccaaccag

SEQ ID NO: 151 - DNA, 19 bp, Artificial Sequence
  antisense oligonucleotide
ccaaggccatctccaacca

SEQ ID NO: 152 - DNA, 19 bp, Artificial Sequence
  antisense oligonucleotide
gctccaaggccatctccaa

SEQ ID NO: 153 - DNA, 19 bp, Artificial Sequence
  antisense oligonucleotide
gaaacttctctaggcctgc

SEQ ID NO: 154 - DNA, 19 bp, Artificial Sequence
  antisense oligonucleotide
ggcatgtgtaaaggtggag

SEQ ID NO: 155 - DNA, 19 bp, Artificial Sequence
  antisense oligonucleotide
cgcttactgcctcagcttc

SEQ ID NO: 156 - DNA, 19 bp, Artificial Sequence
  antisense oligonucleotide
tggaatgcggcggcaggag

SEQ ID NO: 157 - DNA, 19 bp, Artificial Sequence
  antisense oligonucleotide
ggtggaatgcggcggcagg

SEQ ID NO: 158 - DNA, 19 bp, Artificial Sequence
  antisense oligonucleotide
gtgtggatgtgaggagtgg

SEQ ID NO: 159 - DNA, 19 bp, Artificial Sequence
  antisense oligonucleotide
gtgcagtgtgtggatgtga

SEQ ID NO: 160 - DNA, 19 bp, Artificial Sequence
  antisense oligonucleotide
ttagcatgctctcatattt

SEQ ID NO: 161 - DNA, 19 bp, Artificial Sequence
  antisense oligonucleotide
cttagcatgctctcatatt

SEQ ID NO: 162 - DNA, 19 bp, Artificial Sequence
  antisense oligonucleotide
ccttagcatgctctcatat

SEQ ID NO: 163 - DNA, 18 bp, Artificial Sequence
  antisense oligonucleotide
ggactgagagtgaaaggt

SEQ ID NO: 164 - DNA, 18 bp, Artificial Sequence
  antisense oligonucleotide
caaggaccggctgagagg

SEQ ID NO: 165 - DNA, 18 bp, Artificial Sequence
  antisense oligonucleotide
ccaaggaccggctgagag

SEQ ID NO: 166 - DNA, 18 bp, Artificial Sequence
  antisense oligonucleotide
ccgtcattctacagaaac

SEQ ID NO: 167 - DNA, 18 bp, Artificial Sequence
  antisense oligonucleotide
aaggcagagccgccacgc

SEQ ID NO: 168 - DNA, 18 bp, Artificial Sequence
  antisense oligonucleotide
caaggcagagccgccacg

SEQ ID NO: 169 - DNA, 18 bp, Artificial Sequence
  antisense oligonucleotide
agtgaggaccaaggatgc

SEQ ID NO: 170 - DNA, 18 bp, Artificial Sequence
  antisense oligonucleotide
gagtgaggaccaaggatg

SEQ ID NO: 171 - DNA, 18 bp, Artificial Sequence
  antisense oligonucleotide
tcgagtgaggaccaagga

SEQ ID NO: 172 - DNA, 18 bp, Artificial Sequence
  antisense oligonucleotide
aaactcgagtgaggacca

SEQ ID NO: 173 - DNA, 18 bp, Artificial Sequence
  antisense oligonucleotide
taaagccaaggttagtcc

SEQ ID NO: 174 - DNA, 18 bp, Artificial Sequence
  antisense oligonucleotide
gtaaagccaaggttagtc

SEQ ID NO: 175 - DNA, 18 bp, Artificial Sequence
  antisense oligonucleotide
cgtaaagccaaggttagt

SEQ ID NO: 176 - DNA, 18 bp, Artificial Sequence
  antisense oligonucleotide
tcgtaaagccaaggttag

SEQ ID NO: 177 - DNA, 18 bp, Artificial Sequence
  antisense oligonucleotide
actcgaacaggtacactt

SEQ ID NO: 178 - DNA, 18 bp, Artificial Sequence
  antisense oligonucleotide
cactcgaacaggtacact

SEQ ID NO: 179 - DNA, 18 bp, Artificial Sequence
  antisense oligonucleotide
ctcactcgaacaggtaca

SEQ ID NO: 180 - DNA, 18 bp, Artificial Sequence
  antisense oligonucleotide
tgtcctcactcgaacagg

SEQ ID NO: 181 - DNA, 18 bp, Artificial Sequence
  antisense oligonucleotide
actgtcctcactcgaaca

SEQ ID NO: 182 - DNA, 18 bp, Artificial Sequence
  antisense oligonucleotide
aactgtcctcactcgaac

SEQ ID NO: 183 - DNA, 18 bp, Artificial Sequence
  antisense oligonucleotide
gaactgtcctcactcgaa

SEQ ID NO: 184 - DNA, 18 bp, Artificial Sequence
  antisense oligonucleotide
agaactgtcctcactcga

SEQ ID NO: 185 - DNA, 18 bp, Artificial Sequence
  antisense oligonucleotide
aagaactgtcctcactcg

SEQ ID NO: 186 - DNA, 18 bp, Artificial Sequence
  antisense oligonucleotide
acggatggtctgaacagg

SEQ ID NO: 187 - DNA, 18 bp, Artificial Sequence
  antisense oligonucleotide
ccacggatggtctgaaca

SEQ ID NO: 188 - DNA, 18 bp, Artificial Sequence
  antisense oligonucleotide
ggactgtcttaggcttgg

SEQ ID NO: 189 - DNA, 18 bp, Artificial Sequence
  antisense oligonucleotide
aagaggtggcgctgaggc

SEQ ID NO: 190 - DNA, 18 bp, Artificial Sequence
  antisense oligonucleotide
gctgcggacaccttgctc

SEQ ID NO: 191 - DNA, 18 bp, Artificial Sequence
  antisense oligonucleotide
aggctgcggacaccttgc

SEQ ID NO: 192 - DNA, 18 bp, Artificial Sequence
  antisense oligonucleotide
cgagtgtcaggctgcgga

SEQ ID NO: 193 - DNA, 18 bp, Artificial Sequence
  antisense oligonucleotide
tgtccggcacaagcgcgg

SEQ ID NO: 194 - DNA, 18 bp, Artificial Sequence
  antisense oligonucleotide
tgagagtcttgtccggca

SEQ ID NO: 195 - DNA, 18 bp, Artificial Sequence
  antisense oligonucleotide
gtgagagtcttgtccggc

SEQ ID NO: 196 - DNA, 18 bp, Artificial Sequence
  antisense oligonucleotide
ctgtgagagtcttgtccg

SEQ ID NO: 197 - DNA, 18 bp, Artificial Sequence
  antisense oligonucleotide
aggcatcctggaatgaag

SEQ ID NO: 198 - DNA, 18 bp, Artificial Sequence
  antisense oligonucleotide
agtgaatagatcaggagg

SEQ ID NO: 199 - DNA, 18 bp, Artificial Sequence
  antisense oligonucleotide
acgcagtgaatagatcag

SEQ ID NO: 200 - DNA, 18 bp, Artificial Sequence
  antisense oligonucleotide
aacgcagtgaatagatca

SEQ ID NO: 201 - DNA, 18 bp, Artificial Sequence
  antisense oligonucleotide
gaacgcagtgaatagatc

SEQ ID NO: 202 - DNA, 18 bp, Artificial Sequence
  antisense oligonucleotide
tggcgaacgcagtgaata

SEQ ID NO: 203 - DNA, 18 bp, Artificial Sequence
  antisense oligonucleotide
atggcgaacgcagtgaat

SEQ ID NO: 204 - DNA, 18 bp, Artificial Sequence
  antisense oligonucleotide
aatggcgaacgcagtgaa

SEQ ID NO: 205 - DNA, 18 bp, Artificial Sequence
  antisense oligonucleotide
gaatggcgaacgcagtga

SEQ ID NO: 206 - DNA, 18 bp, Artificial Sequence
  antisense oligonucleotide
cgaatggcgaacgcagtg

SEQ ID NO: 207 - DNA, 18 bp, Artificial Sequence
  antisense oligonucleotide
cgctgactggagctcaca

SEQ ID NO: 208 - DNA, 18 bp, Artificial Sequence
  antisense oligonucleotide
acggagaagtcagtaagg

SEQ ID NO: 209 - DNA, 18 bp, Artificial Sequence
  antisense oligonucleotide
taatcctcaggacgcagc

SEQ ID NO: 210 - DNA, 18 bp, Artificial Sequence
  antisense oligonucleotide
tttaatcctcaggacgca

SEQ ID NO: 211 - DNA, 18 bp, Artificial Sequence
  antisense oligonucleotide
gttattgattctaggtga

SEQ ID NO: 212 - DNA, 18 bp, Artificial Sequence
  antisense oligonucleotide
agttattgattctaggtg

SEQ ID NO: 213 - DNA, 18 bp, Artificial Sequence
  antisense oligonucleotide
ccgtgcagagatgaaggc

SEQ ID NO: 214 - DNA, 18 bp, Artificial Sequence
  antisense oligonucleotide
cttcctgtacagagtcac

SEQ ID NO: 215 - DNA, 18 bp, Artificial Sequence
  antisense oligonucleotide
gcttcctgtacagagtca

SEQ ID NO: 216 - DNA, 18 bp, Artificial Sequence
  antisense oligonucleotide
ggactaggtaagatgagg

SEQ ID NO: 217 - DNA, 18 bp, Artificial Sequence
  antisense oligonucleotide
aactcctcacagtcgtgt

SEQ ID NO: 218 - DNA, 18 bp, Artificial Sequence
  antisense oligonucleotide
ggagtgaggtcttccaac

SEQ ID NO: 219 - DNA, 18 bp, Artificial Sequence
  antisense oligonucleotide
gaagcgcacagaagaagg

SEQ ID NO: 220 - DNA, 18 bp, Artificial Sequence
  antisense oligonucleotide
ccagaagcgcacagaaga

SEQ ID NO: 221 - DNA, 18 bp, Artificial Sequence
  antisense oligonucleotide
ctttccagaagcgcacag

SEQ ID NO: 222 - DNA, 18 bp, Artificial Sequence
  antisense oligonucleotide
ccgcattacaggacattg

SEQ ID NO: 223 - DNA, 18 bp, Artificial Sequence
  antisense oligonucleotide
gagagaccgcattacagg

SEQ ID NO: 224 - DNA, 18 bp, Artificial Sequence
  antisense oligonucleotide
agagagaccgcattacag

SEQ ID NO: 225 - DNA, 18 bp, Artificial Sequence
  antisense oligonucleotide
cagagagaccgcattaca

SEQ ID NO: 226 - DNA, 18 bp, Artificial Sequence
  antisense oligonucleotide
gcagagagaccgcattac

SEQ ID NO: 227 - DNA, 18 bp, Artificial Sequence
  antisense oligonucleotide
cctggatcaagttagatt

SEQ ID NO: 228 - DNA, 18 bp, Artificial Sequence
  antisense oligonucleotide
cttggctggtagtgtcta

SEQ ID NO: 229 - DNA, 18 bp, Artificial Sequence
  antisense oligonucleotide
ggtgtctgcagttcaagc

SEQ ID NO: 230 - DNA, 18 bp, Artificial Sequence
  antisense oligonucleotide
ctcctgacttgatctgtg

SEQ ID NO: 231 - DNA, 18 bp, Artificial Sequence
  antisense oligonucleotide
gctcctgacttgatctgt

SEQ ID NO: 232 - DNA, 18 bp, Artificial Sequence
  antisense oligonucleotide
aactggaaacttagctgc

SEQ ID NO: 233 - DNA, 18 bp, Artificial Sequence
  antisense oligonucleotide
gtgaggaacagaagtcat

SEQ ID NO: 234 - DNA, 18 bp, Artificial Sequence
  antisense oligonucleotide
ccttcacctgcgtgcctg

SEQ ID NO: 235 - DNA, 18 bp, Artificial Sequence
  antisense oligonucleotide
cgctcgcctccttcacct

SEQ ID NO: 236 - DNA, 18 bp, Artificial Sequence
  antisense oligonucleotide
gctgacaagcgctcgcct

SEQ ID NO: 237 - DNA, 18 bp, Artificial Sequence
  antisense oligonucleotide
ccaactcctagtgccaac

SEQ ID NO: 238 - DNA, 18 bp, Artificial Sequence
  antisense oligonucleotide
catgtgaccaggaccaac

SEQ ID NO: 239 - DNA, 18 bp, Artificial Sequence
  antisense oligonucleotide
ctcaaggagcaaccagct

SEQ ID NO: 240 - DNA, 18 bp, Artificial Sequence
  antisense oligonucleotide
tgactcaaggagcaacca

SEQ ID NO: 241 - DNA, 18 bp, Artificial Sequence
  antisense oligonucleotide
atgactcaaggagcaacc

SEQ ID NO: 242 - DNA, 18 bp, Artificial Sequence
  antisense oligonucleotide
gcggtggtgatgactcaa

SEQ ID NO: 243 - DNA, 18 bp, Artificial Sequence
  antisense oligonucleotide
cagcgaggtgacacagag

SEQ ID NO: 244 - DNA, 18 bp, Artificial Sequence
  antisense oligonucleotide
cactcagttccgtctcag

SEQ ID NO: 245 - DNA, 18 bp, Artificial Sequence
  antisense oligonucleotide
tcactcagttccgtctca

SEQ ID NO: 246 - DNA, 18 bp, Artificial Sequence
  antisense oligonucleotide
tgtcactcagttccgtct

SEQ ID NO: 247 - DNA, 18 bp, Artificial Sequence
  antisense oligonucleotide
ctgtcactcagttccgtc

SEQ ID NO: 248 - DNA, 18 bp, Artificial Sequence
  antisense oligonucleotide
ccgagatgccatgcaacg

SEQ ID NO: 249 - DNA, 18 bp, Artificial Sequence
  antisense oligonucleotide
ccaacacaggcgcttaag

SEQ ID NO: 250 - DNA, 18 bp, Artificial Sequence
  antisense oligonucleotide
caccaacacaggcgctta

SEQ ID NO: 251 - DNA, 18 bp, Artificial Sequence
  antisense oligonucleotide
tgaaacatatgcctgcca

SEQ ID NO: 252 - DNA, 18 bp, Artificial Sequence
  antisense oligonucleotide
gacctcctgaaacatatg

SEQ ID NO: 253 - DNA, 18 bp, Artificial Sequence
  antisense oligonucleotide
tcggatgtggacagacac

SEQ ID NO: 254 - DNA, 18 bp, Artificial Sequence
  antisense oligonucleotide
tgactcggatgtggacag

SEQ ID NO: 255 - DNA, 18 bp, Artificial Sequence
  antisense oligonucleotide
ttgactcggatgtggaca

SEQ ID NO: 256 - DNA, 18 bp, Artificial Sequence
  antisense oligonucleotide
atgcttcagagacgagat

SEQ ID NO: 257 - DNA, 18 bp, Artificial Sequence
  antisense oligonucleotide
gactagagctcacagcaa

SEQ ID NO: 258 - DNA, 18 bp, Artificial Sequence
  antisense oligonucleotide
tttcaggacaagctcgga

SEQ ID NO: 259 - DNA, 18 bp, Artificial Sequence
  antisense oligonucleotide
cctactaagagccttcac

SEQ ID NO: 260 - DNA, 18 bp, Artificial Sequence
  antisense oligonucleotide
ccttctacgtgaggctgc

SEQ ID NO: 261 - DNA, 18 bp, Artificial Sequence
  antisense oligonucleotide
tccttctacgtgaggctg

SEQ ID NO: 262 - DNA, 18 bp, Artificial Sequence
  antisense oligonucleotide
cctcttccttctacgtga

SEQ ID NO: 263 - DNA, 18 bp, Artificial Sequence
  antisense oligonucleotide
ggaatgtcattgagaagt

SEQ ID NO: 264 - DNA, 18 bp, Artificial Sequence
  antisense oligonucleotide
cagagatgccggtcacca

SEQ ID NO: 265 - DNA, 18 bp, Artificial Sequence
  antisense oligonucleotide
ctagaggacagagatgcc

SEQ ID NO: 266 - DNA, 18 bp, Artificial Sequence
  antisense oligonucleotide
agccgtgctcctaggtgg

SEQ ID NO: 267 - DNA, 18 bp, Artificial Sequence
  antisense oligonucleotide
agtggatcatgcaggaaa

SEQ ID NO: 268 - DNA, 18 bp, Artificial Sequence
  antisense oligonucleotide
ccgcaggcaggcacatat

SEQ ID NO: 269 - DNA, 18 bp, Artificial Sequence
  antisense oligonucleotide
gtctccaatgtaagataa

SEQ ID NO: 270 - DNA, 19 bp, Artificial Sequence
  antisense oligonucleotide
tctacagaaacacgcagcc

SEQ ID NO: 271 - DNA, 19 bp, Artificial Sequence
  antisense oligonucleotide
ccgtcattctacagaaaca

SEQ ID NO: 272 - DNA, 19 bp, Artificial Sequence
  antisense oligonucleotide
aaggcagagccgccacgca

SEQ ID NO: 273 - DNA, 19 bp, Artificial Sequence
  antisense oligonucleotide
tcgtaaagccaaggttagt

SEQ ID NO: 274 - DNA, 19 bp, Artificial Sequence
  antisense oligonucleotide
ctcatctaaactttgacgt

SEQ ID NO: 275 - DNA, 19 bp, Artificial Sequence
  antisense oligonucleotide
caccagctcatctaaactt

SEQ ID NO: 276 - DNA, 19 bp, Artificial Sequence
  antisense oligonucleotide
ctcactcgaacaggtacac

SEQ ID NO: 277 - DNA, 19 bp, Artificial Sequence
  antisense oligonucleotide
agaactgtcctcactcgaa

SEQ ID NO: 278 - DNA, 19 bp, Artificial Sequence
  antisense oligonucleotide
aagaactgtcctcactcga

SEQ ID NO: 279 - DNA, 19 bp, Artificial Sequence
  antisense oligonucleotide
gaagaactgtcctcactcg

SEQ ID NO: 280 - DNA, 19 bp, Artificial Sequence
  antisense oligonucleotide
cacggatggtctgaacagg

SEQ ID NO: 281 - DNA, 19 bp, Artificial Sequence
  antisense oligonucleotide
ggactgtcttaggcttggc

SEQ ID NO: 282 - DNA, 19 bp, Artificial Sequence
  antisense oligonucleotide
aaggatgctgcctcaggtg

SEQ ID NO: 283 - DNA, 19 bp, Artificial Sequence
  antisense oligonucleotide
ctgacaaggatgctgcctc

SEQ ID NO: 284 - DNA, 19 bp, Artificial Sequence
  antisense oligonucleotide
aagaggtggcgctgaggca

SEQ ID NO: 285 - DNA, 19 bp, Artificial Sequence
  antisense oligonucleotide
gaatctccagttctgagtc

SEQ ID NO: 286 - DNA, 19 bp, Artificial Sequence
  antisense oligonucleotide
agaatctccagttctgagt

SEQ ID NO: 287 - DNA, 19 bp, Artificial Sequence
  antisense oligonucleotide
gtgagagtcttgtccggca

SEQ ID NO: 288 - DNA, 19 bp, Artificial Sequence
  antisense oligonucleotide
tgtgagagtcttgtccggc

SEQ ID NO: 289 - DNA, 19 bp, Artificial Sequence
  antisense oligonucleotide
cctgtgagagtcttgtccg

SEQ ID NO: 290 - DNA, 19 bp, Artificial Sequence
  antisense oligonucleotide
tgccacctgtgagagtctt

SEQ ID NO: 291 - DNA, 19 bp, Artificial Sequence
  antisense oligonucleotide
aggcatcctggaatgaaga

SEQ ID NO: 292 - DNA, 19 bp, Artificial Sequence
  antisense oligonucleotide
gaggcatcctggaatgaag

SEQ ID NO: 293 - DNA, 19 bp, Artificial Sequence
  antisense oligonucleotide
aacgcagtgaatagatcag

SEQ ID NO: 294 - DNA, 19 bp, Artificial Sequence
  antisense oligonucleotide
cgaatggcgaacgcagtga

SEQ ID NO: 295 - DNA, 19 bp, Artificial Sequence
  antisense oligonucleotide
ctcttatttatgctcctgc

SEQ ID NO: 296 - DNA, 19 bp, Artificial Sequence
  antisense oligonucleotide
agcagaggctcttatttat

SEQ ID NO: 297 - DNA, 19 bp, Artificial Sequence
  antisense oligonucleotide
cacggagaagtcagtaagg

SEQ ID NO: 298 - DNA, 19 bp, Artificial Sequence
  antisense oligonucleotide
aagcacacggagaagtcag

SEQ ID NO: 299 - DNA, 19 bp, Artificial Sequence
  antisense oligonucleotide
taatcctcaggacgcagcc

SEQ ID NO: 300 - DNA, 19 bp, Artificial Sequence
  antisense oligonucleotide
gtcatggaggccagatgca

SEQ ID NO: 301 - DNA, 19 bp, Artificial Sequence
  antisense oligonucleotide
tgtcatggaggccagatgc

SEQ ID NO: 302 - DNA, 19 bp, Artificial Sequence
  antisense oligonucleotide
agttattgattctaggtga

SEQ ID NO: 303 - DNA, 19 bp, Artificial Sequence
  antisense oligonucleotide
gctccatcctgcacgtcca

SEQ ID NO: 304 - DNA, 19 bp, Artificial Sequence
  antisense oligonucleotide
ccgtgcagagatgaaggca

SEQ ID NO: 305 - DNA, 19 bp, Artificial Sequence
  antisense oligonucleotide
gcttcctgtacagagtcac

SEQ ID NO: 306 - DNA, 19 bp, Artificial Sequence
  antisense oligonucleotide
caactcctcacagtcgtgt

SEQ ID NO: 307 - DNA, 19 bp, Artificial Sequence
  antisense oligonucleotide
gtcttccaactcctcacag

SEQ ID NO: 308 - DNA, 19 bp, Artificial Sequence
  antisense oligonucleotide
agaagcgcacagaagaagg

SEQ ID NO: 309 - DNA, 19 bp, Artificial Sequence
  antisense oligonucleotide
ccagaagcgcacagaagaa

SEQ ID NO: 310 - DNA, 19 bp, Artificial Sequence
  antisense oligonucleotide
ttccagaagcgcacagaag

SEQ ID NO: 311 - DNA, 19 bp, Artificial Sequence
  antisense oligonucleotide
tttccagaagcgcacagaa

SEQ ID NO: 312 - DNA, 19 bp, Artificial Sequence
  antisense oligonucleotide
ctttccagaagcgcacaga

SEQ ID NO: 313 - DNA, 19 bp, Artificial Sequence
  antisense oligonucleotide
cattacaggacattgcttt

SEQ ID NO: 314 - DNA, 19 bp, Artificial Sequence
  antisense oligonucleotide
cagagagaccgcattacag

SEQ ID NO: 315 - DNA, 19 bp, Artificial Sequence
  antisense oligonucleotide
cgcagagagaccgcattac

SEQ ID NO: 316 - DNA, 19 bp, Artificial Sequence
  antisense oligonucleotide
cctggatcaagttagattt

SEQ ID NO: 317 - DNA, 19 bp, Artificial Sequence
  antisense oligonucleotide
aaggtgaatattcagaagt

SEQ ID NO: 318 - DNA, 19 bp, Artificial Sequence
  antisense oligonucleotide
aggaaggtgaatattcaga

SEQ ID NO: 319 - DNA, 19 bp, Artificial Sequence
  antisense oligonucleotide
tctactaggaaggtgaata

SEQ ID NO: 320 - DNA, 19 bp, Artificial Sequence
  antisense oligonucleotide
ccttggctggtagtgtcta

SEQ ID NO: 321 - DNA, 19 bp, Artificial Sequence
  antisense oligonucleotide
tgcctccaccaactggctg

SEQ ID NO: 322 - DNA, 19 bp, Artificial Sequence
  antisense oligonucleotide
ctgcctccaccaactggct

SEQ ID NO: 323 - DNA, 19 bp, Artificial Sequence
  antisense oligonucleotide
cctgacttgatctgtgctg

SEQ ID NO: 324 - DNA, 19 bp, Artificial Sequence
  antisense oligonucleotide
ctcctgacttgatctgtgc

SEQ ID NO: 325 - DNA, 19 bp, Artificial Sequence
  antisense oligonucleotide
gctcctgacttgatctgtg

SEQ ID NO: 326 - DNA, 19 bp, Artificial Sequence
  antisense oligonucleotide
tgctcctgacttgatctgt

SEQ ID NO: 327 - DNA, 19 bp, Artificial Sequence
  antisense oligonucleotide
tttgcacacattggtggag

SEQ ID NO: 328 - DNA, 19 bp, Artificial Sequence
  antisense oligonucleotide
atttgcacacattggtgga

SEQ ID NO: 329 - DNA, 19 bp, Artificial Sequence
  antisense oligonucleotide
ctgctatttatttgcacac

SEQ ID NO: 330 - DNA, 19 bp, Artificial Sequence
  antisense oligonucleotide
ggaaacttagctgctattt

SEQ ID NO: 331 - DNA, 19 bp, Artificial Sequence
  antisense oligonucleotide
atctgtgcattgttcttgt

SEQ ID NO: 332 - DNA, 19 bp, Artificial Sequence
  antisense oligonucleotide
catctgtgcattgttcttg

SEQ ID NO: 333 - DNA, 19 bp, Artificial Sequence
  antisense oligonucleotide
cgacaggacaatggccgct

SEQ ID NO: 334 - DNA, 19 bp, Artificial Sequence
  antisense oligonucleotide
tggctaggaagtgctaagg

SEQ ID NO: 335 - DNA, 19 bp, Artificial Sequence
  antisense oligonucleotide
aggaacagaagtcatcacg

SEQ ID NO: 336 - DNA, 19 bp, Artificial Sequence
  antisense oligonucleotide
gtgaggaacagaagtcatc

SEQ ID NO: 337 - DNA, 19 bp, Artificial Sequence
  antisense oligonucleotide
ggtgaggaacagaagtcat

SEQ ID NO: 338 - DNA, 19 bp, Artificial Sequence
  antisense oligonucleotide
gcaggtgaggaacagaagt

SEQ ID NO: 339 - DNA, 19 bp, Artificial Sequence
  antisense oligonucleotide
acaggcaggtgaggaacag

SEQ ID NO: 340 - DNA, 19 bp, Artificial Sequence
  antisense oligonucleotide
ggactcggcacagagcagg

SEQ ID NO: 341 - DNA, 19 bp, Artificial Sequence
  antisense oligonucleotide
gcctgaatggaggaagatg

SEQ ID NO: 342 - DNA, 19 bp, Artificial Sequence
  antisense oligonucleotide
gctcgcctccttcacctgc

SEQ ID NO: 343 - DNA, 19 bp, Artificial Sequence
  antisense oligonucleotide
cgctcgcctccttcacctg

SEQ ID NO: 344 - DNA, 19 bp, Artificial Sequence
  antisense oligonucleotide
ggtttgaagtgaccttgag

SEQ ID NO: 345 - DNA, 19 bp, Artificial Sequence
  antisense oligonucleotide
cctagtgccaacctcactg

SEQ ID NO: 346 - DNA, 19 bp, Artificial Sequence
  antisense oligonucleotide
tgaccaggaccaactccta

SEQ ID NO: 347 - DNA, 19 bp, Artificial Sequence
  antisense oligonucleotide
agcaaccagctcagaggag

SEQ ID NO: 348 - DNA, 19 bp, Artificial Sequence
  antisense oligonucleotide
tgactcaaggagcaaccag

SEQ ID NO: 349 - DNA, 19 bp, Artificial Sequence
  antisense oligonucleotide
atgactcaaggagcaacca

SEQ ID NO: 350 - DNA, 19 bp, Artificial Sequence
  antisense oligonucleotide
ggcggtggtgatgactcaa

SEQ ID NO: 351 - DNA, 19 bp, Artificial Sequence
  antisense oligonucleotide
ggtgacacagagaccaggc

SEQ ID NO: 352 - DNA, 19 bp, Artificial Sequence
  antisense oligonucleotide
cgaggtgacacagagacca

SEQ ID NO: 353 - DNA, 19 bp, Artificial Sequence
  antisense oligonucleotide
cagcgaggtgacacagaga

SEQ ID NO: 354 - DNA, 19 bp, Artificial Sequence
  antisense oligonucleotide
ccagcgaggtgacacagag

SEQ ID NO: 355 - DNA, 19 bp, Artificial Sequence
  antisense oligonucleotide
cactcagttccgtctcagg

SEQ ID NO: 356 - DNA, 19 bp, Artificial Sequence
  antisense oligonucleotide
tcactcagttccgtctcag

SEQ ID NO: 357 - DNA, 19 bp, Artificial Sequence
  antisense oligonucleotide
tgtcactcagttccgtctc

SEQ ID NO: 358 - DNA, 19 bp, Artificial Sequence
  antisense oligonucleotide
ctgtcactcagttccgtct

SEQ ID NO: 359 - DNA, 19 bp, Artificial Sequence
  antisense oligonucleotide
gcaccaacacaggcgctta

SEQ ID NO: 360 - DNA, 19 bp, Artificial Sequence
  antisense oligonucleotide
ggaggcaccaacacaggcg

SEQ ID NO: 361 - DNA, 19 bp, Artificial Sequence
  antisense oligonucleotide
ctgaaacatatgcctgcca

SEQ ID NO: 362 - DNA, 19 bp, Artificial Sequence
  antisense oligonucleotide
cctgaaacatatgcctgcc

SEQ ID NO: 363 - DNA, 19 bp, Artificial Sequence
  antisense oligonucleotide
gacctcctgaaacatatgc

SEQ ID NO: 364 - DNA, 19 bp, Artificial Sequence
  antisense oligonucleotide
acaaggacctcctgaaaca

SEQ ID NO: 365 - DNA, 19 bp, Artificial Sequence
  antisense oligonucleotide
ccaagacaaggacctcctg

SEQ ID NO: 366 - DNA, 19 bp, Artificial Sequence
  antisense oligonucleotide
actcggatgtggacagaca

SEQ ID NO: 367 - DNA, 19 bp, Artificial Sequence
  antisense oligonucleotide
tgactcggatgtggacaga

SEQ ID NO: 368 - DNA, 19 bp, Artificial Sequence
  antisense oligonucleotide
attgactcggatgtggaca

SEQ ID NO: 369 - DNA, 19 bp, Artificial Sequence
  antisense oligonucleotide
cattgactcggatgtggac

SEQ ID NO: 370 - DNA, 19 bp, Artificial Sequence
  antisense oligonucleotide
atgcttcagagacgagatg

SEQ ID NO: 371 - DNA, 19 bp, Artificial Sequence
  antisense oligonucleotide
gatgcttcagagacgagat

SEQ ID NO: 372 - DNA, 19 bp, Artificial Sequence
  antisense oligonucleotide
gcaaagatgcttcagagac

SEQ ID NO: 373 - DNA, 19 bp, Artificial Sequence
  antisense oligonucleotide
tagagctcacagcaaagat

SEQ ID NO: 374 - DNA, 19 bp, Artificial Sequence
  antisense oligonucleotide
ctagagctcacagcaaaga

SEQ ID NO: 375 - DNA, 19 bp, Artificial Sequence
  antisense oligonucleotide
actagagctcacagcaaag

SEQ ID NO: 376 - DNA, 19 bp, Artificial Sequence
  antisense oligonucleotide
gactagagctcacagcaaa

SEQ ID NO: 377 - DNA, 19 bp, Artificial Sequence
  antisense oligonucleotide
ggactagagctcacagcaa

SEQ ID NO: 378 - DNA, 19 bp, Artificial Sequence
  antisense oligonucleotide
cctgtctgcactgctctgg

SEQ ID NO: 379 - DNA, 19 bp, Artificial Sequence
  antisense oligonucleotide
cctgatttcctactaagag

SEQ ID NO: 380 - DNA, 19 bp, Artificial Sequence
  antisense oligonucleotide
ttccttctacgtgaggctg

SEQ ID NO: 381 - DNA, 19 bp, Artificial Sequence
  antisense oligonucleotide
gcctcttccttctacgtga

SEQ ID NO: 382 - DNA, 19 bp, Artificial Sequence
  antisense oligonucleotide
tgcagagcctcttccttct

SEQ ID NO: 383 - DNA, 19 bp, Artificial Sequence
  antisense oligonucleotide
tcattgagaagtctctgct

SEQ ID NO: 384 - DNA, 19 bp, Artificial Sequence
  antisense oligonucleotide
gtcattgagaagtctctgc

SEQ ID NO: 385 - DNA, 19 bp, Artificial Sequence
  antisense oligonucleotide
agctggaatgtcattgaga

SEQ ID NO: 386 - DNA, 19 bp, Artificial Sequence
  antisense oligonucleotide
ctagaggacagagatgccg

SEQ ID NO: 387 - DNA, 19 bp, Artificial Sequence
  antisense oligonucleotide
gctagaggacagagatgcc

SEQ ID NO: 388 - DNA, 19 bp, Artificial Sequence
  antisense oligonucleotide
cagagctagaggacagaga

SEQ ID NO: 389 - DNA, 19 bp, Artificial Sequence
  antisense oligonucleotide
cagtggatcatgcaggaaa

SEQ ID NO: 390 - DNA, 19 bp, Artificial Sequence
  antisense oligonucleotide
tataatagaatgtgagtcc

SEQ ID NO: 391 - DNA, 19 bp, Artificial Sequence
  antisense oligonucleotide
ccgcaggcaggcacatatg

SEQ ID NO: 392 - DNA, 19 bp, Artificial Sequence
  antisense oligonucleotide
gataagaaatgaccaagcc

SEQ ID NO: 393 - DNA, 19 bp, Artificial Sequence
  antisense oligonucleotide
tgtaagataagaaatgacc

SEQ ID NO: 394 - DNA, 19 bp, Artificial Sequence
  antisense oligonucleotide
ctcctgtctccaatgtaag

SEQ ID NO: 395 - DNA, 19 bp, Artificial Sequence
  antisense oligonucleotide
ctctcctgtctccaatgta

SEQ ID NO: 396 - DNA, 19 bp, Artificial Sequence
  antisense oligonucleotide
gctctcctgtctccaatgt

SEQ ID NO: 397 - DNA, 19 bp, Artificial Sequence
  antisense oligonucleotide
agctctcctgtctccaatg

SEQ ID NO: 398 - DNA, 19 bp, Artificial Sequence
  antisense oligonucleotide
aagctctcctgtctccaat

SEQ ID NO: 399 - DNA, 19 bp, Artificial Sequence
  antisense oligonucleotide
caagctctcctgtctccaa

SEQ ID NO: 400 - DNA, 19 bp, Artificial Sequence
  antisense oligonucleotide
tcaagctctcctgtctcca

SEQ ID NO: 401 - DNA, 19 bp, Artificial Sequence
  antisense oligonucleotide
tcttgcagatttaggattc

SEQ ID NO: 402 - DNA, 19 bp, Artificial Sequence
  antisense oligonucleotide
ttcttgcagatttaggatt

SEQ ID NO: 403 - DNA, 19 bp, Artificial Sequence
  antisense oligonucleotide
tggcattcttgcagattta

SEQ ID NO: 404 - DNA, 19 bp, Artificial Sequence
  antisense oligonucleotide
ctggcattcttgcagattt

SEQ ID NO: 405 - DNA, 19 bp, Artificial Sequence
  antisense oligonucleotide
cctggcattcttgcagatt

SEQ ID NO: 406 - DNA, 18 bp, Artificial Sequence
  negative control
cgtttaggctatgtactt

SEQ ID NO: 407 - DNA, 17 bp, Artificial Sequence
  negative control
gatcattcgcggacaac

SEQ ID NO: 408 - DNA, 17 bp, Artificial Sequence
  negative control
gactcgttaaaccgata